(19) United States
(12) Patent Application Publication
(10) Pub. No.: US 2019/0161485 A1
(43) Pub. Date: May 30, 2019

(54) ORGANIC COMPOUND, ORGANIC LIGHT EMITTING DIODE INCLUDING THE SAME AND DISPLAY INCLUDING THE ORGANIC LIGHT EMITTING DIODE

(71) Applicant: SOULBRAIN CO., LTD., Seongnam-si (KR)

(72) Inventors: Jong Jin HA, Seongnam-si (KR); Hyun Young YOO, Seongnam-si (KR); Sung Soo KIM, Seongnam-si (KR); Seok Jong LEE, Seongnam-si (KR)

(21) Appl. No.: 16/198,875

(22) Filed: Nov. 22, 2018

(30) Foreign Application Priority Data
Nov. 30, 2017 (KR) 10-2017-0163448

(51) Int. Cl. C07D 471/04; C07D 519/00; H01L 51/00

(52) U.S. Cl. CPC C07D 471/04; C07D 519/00; H01L 51/5056; H01L 51/0054; H01L 51/0072

(57) ABSTRACT

The present invention provides an organic compound represented by Chemical Formula 1 below:

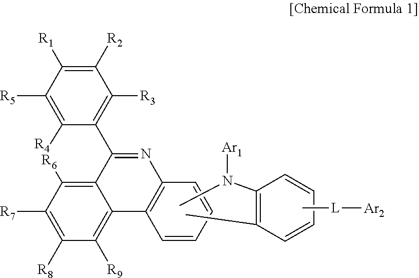

In Chemical Formula 1, R₁ to R₉ are each, independently, any one selected from the group consisting of hydrogen, a halogen, a nitro group, a substituted or unsubstituted C1-C6 alkyl group, a substituted or unsubstituted C3-C6 cycloalkyl group, a substituted or unsubstituted C6-C40 aryl group, and a substituted or unsubstituted C5-C40 heteroaryl group; L represents a single bond and is any one selected from the group consisting of an alkylene group, a cycloalkylene group, a heterocycloalkylene group, an arylene group, and a heteroarylene group; and Ar₁ and Ar₂ are each, independently, any one selected from the group consisting of hydrogen, a substituted or unsubstituted C3-C6 cycloalkyl group, a substituted or unsubstituted C3-C6 heterocycloalkyl group, a substituted or unsubstituted C6-C40 aryl group, and a substituted or unsubstituted C2-C40 heteroaryl group.

According to the present invention, the light emitting efficiency, stability, and lifetime of an organic light emitting diode may be improved.

### CROSS-REFERENCE TO RELATED APPLICATIONS

This application claims priority under 35 U.S.C. § 119 to Korean Patent Application No. 10-2017-0163448, filed on Nov. 30, 2017, in the Korean Intellectual Property Office, the disclosure of which is incorporated herein by reference in its entirety.

### TECHNICAL FIELD

The following disclosure relates to an organic compound capable of improving light emission efficiency, stability and lifetime of an organic light emitting diode, an organic light emitting diode including the same, and a display including the organic light emitting diode.

### BACKGROUND

In recent years, as display devices have become larger in size, there has been increasing demand for a flat display which occupies less space. As one of such flat display technologies, the technology of organic light emitting devices, also referred to as organic light emitting diodes (OLED), is rapidly developing, and several products have already been announced. Organic light emitting diodes are devices that convert electric energy into light by applying an electric current to an organic emitting material, and they have a structure in which an organic material layer is interposed between an anode and a cathode, wherein the organic material layer may be formed with a multilayer structure comprising a hole injecting layer, a hole transport layer, an emitting layer, an electron transport layer, an electron injecting layer, and the like.

When a voltage is applied between the two electrodes of the organic light emitting diode, holes from the anode and electrons from the cathode are injected into the organic layer, and in a process in which the injected holes and electrons are recombined, excitons with high energy are formed. Here, when the formed excitons return to the ground state, light having a specific wavelength is generated.

Meanwhile, in order to increase the efficiency of the light emission and to improve the stability and lifetime of the organic light emitting diode, hole injecting material, hole transport material, emitting material, electron transport material, electron injecting material, and the like, which constitute the organic material layer, should first be supported by the use of stable and efficient materials. However, the development of such organic layer materials has not yet been sufficiently achieved. Therefore, there is a demand for the continuous development of new materials, and in particular, there is a need for the development of hole transport materials and electron transport materials.
Patent Literature: KR2010-0108924

### SUMMARY

To solve the aforementioned problems with existing technology, an objective of the present invention is to provide an organic compound capable of improving the light emission efficiency, stability and lifetime of an organic light emitting diode. Another objective of the present invention is directed at utilizing the organic compound to provide an organic light emitting diode with improved light emission efficiency, stability, and lifetime, and a display including the organic light emitting diode.

The objects of the present invention can be achieved by the embodiments described herein.

To achieve the objects of the present invention, the present invention provides an organic compound represented by Chemical Formula 1 below:

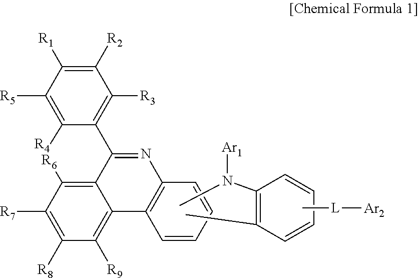

In Chemical Formula 1, R₁ to R₉ are each, independently, any one selected from the group consisting of hydrogen, a halogen, a nitro group, a substituted or unsubstituted C1-C6 alkyl group, a substituted or unsubstituted C3-C6 cycloalkyl group, a substituted or unsubstituted C6-C40 aryl group, and a substituted or unsubstituted C5-C40 heteroaryl group; L represents a single bond, and is any one selected from the group consisting of an alkylene group, a cycloalkylene group, a heterocycloalkylene group, an arylene group, and a heteroarylene group; and Ar₁ and Ar₂ are each, independently, any one selected from the group consisting of hydrogen, a substituted or unsubstituted C3-C6 cycloalkyl group, a substituted or unsubstituted C3-C6 heterocycloalkyl group, a substituted or unsubstituted C6-C40 aryl group, and a substituted or unsubstituted C2-C40 heteroaryl group.

Further, the present invention provides a charge transport material including the organic compound, an organic light emitting diode, and a display.

### DETAILED DESCRIPTION OF EMBODIMENTS

Hereinafter, an organic compound, an organic light emitting diode including the same, and a display including the organic light emitting diode according to the present description are described in detail.

Unless otherwise specified in the present specification, the term “substitution” means that at least one hydrogen in a substituent group or in a compound is replaced with a halogen group, a hydroxyl group, an amino group, a fluorenyl group, a substituted or unsubstituted C1-C30 amine group, a nitro group, a substituted or unsubstituted C1-C40 silyl group, a C1-C30 alkyl group, a C1-C10 alkylsilyl group, a C3-C30 cycloalkyl group, a C6-C30 aryl group, a C1-C20 alkoxy group, a fluoro group, a C1-C10 trifluoroalkyl group such as a trifluoromethyl group or the like, a cyano group, or the like.

Unless otherwise specified in the present specification, the term “hetero” means that 1 to 3 heteroatoms selected from the group consisting of N, O, S and P, are contained in one functional group, while the remainders are carbon atoms.

As used herein, “*-” means a moiety bonded to a carbon atom or an atom other than carbon, that is, a moiety to which a substituent group or a functional group is bonded.

Unless otherwise specified in the present specification, the term “alkyl group” means a saturated aliphatic hydrocarbon group.

As used herein, the term “aryl group” means a substituent group in which all the elements of the cyclic substituent group have a p-orbital shape and these p-orbitals form a conjugation, and the term comprises both monocyclic or fused-ring polycyclic (i.e., a ring with adjacent pairs of divided carbon atoms) functional groups.

As used herein, unless otherwise defined, “heteroaryl” means that 1 to 3 heteroatoms selected from the group consisting of N, O, S and P, are contained in an aryl group, while the remainders are carbon atoms. When the heteroaryl group is a fused ring, 1 to 3 heteroatoms may be included in each ring.

Unless otherwise specified in the present specification, the term “alkylene” is a divalent atomic group obtained by subtracting two hydrogen atoms from an alkane, and it may be represented by the general formula —CnH₂n-. The alkane comprises both linear and branched alkanes, and further comprises primary, secondary, and tertiary alkanes.

Unless otherwise stated in the specification, the term “cycloalkylene” is a divalent atomic group obtained by subtracting two hydrogen atoms from a cycloalkane. The cycloalkane comprises monocyclic or polycyclic cycloalkanes. Further, the cycloalkane may comprise a polycyclic cycloalkyl group including an adamantyl group or a norbornyl group.

Unless otherwise stated in the specification, the term “arylene” means a divalent atomic group obtained by subtracting two hydrogen atoms from an aromatic hydrocarbon, wherein the term aromatic hydrocarbon indicates a monocyclic or polycyclic compound having 6 to 30 carbon atoms and including one or more benzene rings, and a derivative thereof. For example, the arylene may be a benzene ring, toluene or xylene having an alkyl side chain bonded to the benzene ring, biphenyl, in which two or more benzene rings are bonded with a single bond, fluorene, xanthene or anthraquinone, in which the benzene ring is condensed with a cycloalkyl group or a heterocycloalkyl group, naphthalene or anthracene, in which two or more benzene rings are condensed, and the like.

Unless otherwise stated in the specification, the term “charge transport material” refers to an organic material that is responsible for the injection and transportation of charges which are required for light emission, such as an electron or a hole, among the components constituting the organic light emitting diode.

The organic compound of the present invention is represented by Chemical Formula 1 below.

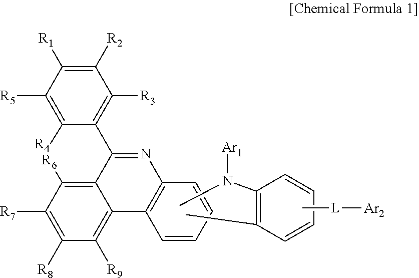

(In Chemical Formula 1, R₁ to R₉ are each, independently, any one selected from the group consisting of hydrogen, a halogen, a nitro group, a substituted or unsubstituted C1-C6 alkyl group, a substituted or unsubstituted C3-C6 cycloalkyl group, a substituted or unsubstituted C6-C40 aryl group, and a substituted or unsubstituted C5-C40 heteroaryl group; L represents a single bond, and is any one selected from the group consisting of an alkylene group, a cycloalkylene group, a heterocycloalkylene group, an arylene group, and a heteroarylene group; and Ar₁ and Ar₂ are each, independently, any one selected from the group consisting of hydrogen, a substituted or unsubstituted C3-C6 cycloalkyl group, a substituted or unsubstituted C3-C6 heterocycloalkyl group, a substituted or unsubstituted C6-C40 aryl group, and a substituted or unsubstituted C2-C40 heteroaryl group).

As a more specific example, R₁ to R₉ may each, independently, be any one selected from the group consisting of hydrogen, a halogen, a nitro group, and a substituted or unsubstituted C1-C6 alkyl group. In this case, excellent charge transport effect, stability and the like may be achieved.

L, representing a single bond, may be, for example, any one selected from the group consisting of an arylene group, and a heteroarylene group. In this case, excellent charge transport effect, stability and the like may be achieved.

As another example, L may be selected from the group consisting of a phenylene group, a naphthylene group, a fluorenylene group, a phenanthrenylene group, an anthracenylene group, a fluoranthrenylene group, a triphenylenylene group, a pyrenylene group, and a chrysenylene group. However, the present invention is not limited thereto.

Ar₁ may be, for example, any one selected from the group consisting of a phenyl group, a biphenyl group, a terphenyl group, a naphthyl group, an anthracenyl group, a phenanthrene group, a pyrenyl group, a triphenylene group, a perylenyl group, a chrysenyl group, a carbazole group, a thiophene group, a furan group, a pyrrolyl group, an imidazole group, a thiazole group, an oxazole group, an oxadiazole group, a triazole group, a pyridyl group, a pyradazine group, a quinolinyl group, an isoquinoline group, and an acridyl group. As a more specific example, Ar₁ may be selected from the group consisting of a phenyl group, a biphenyl group, a terphenyl group, and a naphthyl group, but it is not limited thereto.

Ar₂ is, for example, a pyridyl group, a quinolinyl group, an isoquinolinyl group, a benzoquinolinyl group, a quinoxanyl group, a naphthyridyl group, a pyrazinyl group, a pyrimidyl group, a pyridazinyl group, a phenanthrolyl group, an imidazopyridyl group, a triazyl group, an acridinyl group, an imidazolyl group, a benzimidazolyl group, an oxazolyl group, a benzoxazolyl group, a thiazolyl group, or a benzothiazolyl group, or

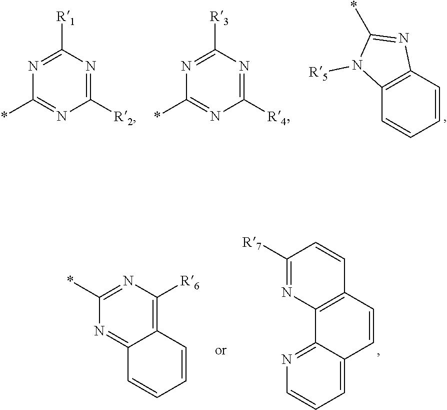

wherein R′₁ to R′₇ are each, independently, any one selected from the group consisting of hydrogen, a halogen, CN, Si(CH)₃, CF₃, nitro, a substituted or unsubstituted C1-C10 alkyl group, a substituted or unsubstituted C3-C10 cycloalkyl group, a substituted or unsubstituted C1-C10 alkoxy group, a phenyl group, a biphenyl group, a terphenyl group, a naphthyl group, an anthracenyl group, a phenanthrene group, a pyrenyl group, a triphenylene group, a perylenyl group, a chrysenyl group, a carbazole group, a thiophene group, a furan group, a pyrrolyl group, an imidazole group, a thiazole group, an oxazole group, an oxadiazole group, a triazole group, a pyridyl group, a pyradazine group, a quinolinyl group, an isoquinoline group, and an acridyl group.

As a more specific example, Ar₂ may be

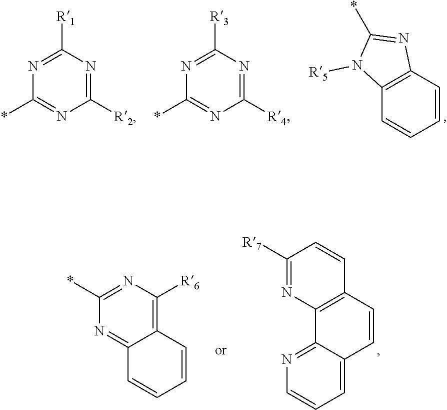

wherein R′₁ and R′₂ are each, independently, any one selected from the group consisting of hydrogen, a substituted or unsubstituted C1-C10 alkyl group, a substituted or unsubstituted C3-C10 cycloalkyl group, a phenyl group, a biphenyl group, a terphenyl group, a naphthyl group, an anthracenyl group, a phenanthrene group, a pyrenyl group, a triphenylene group, and a perylenyl group.

As another example, the compound represented by Chemical Formula 1 above may be a compound represented by any of the following structural formulas 1 to 224, and in this case, it may have excellent charge transport ability, stability, and efficiency.

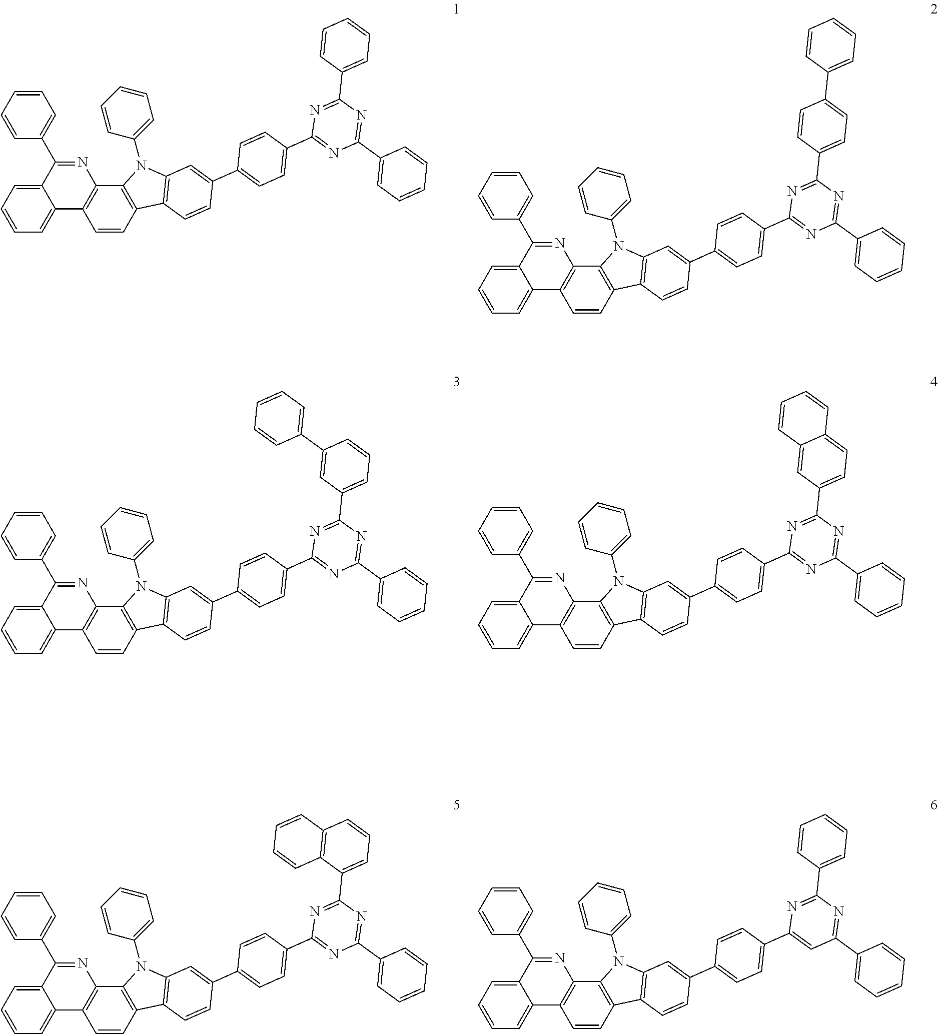

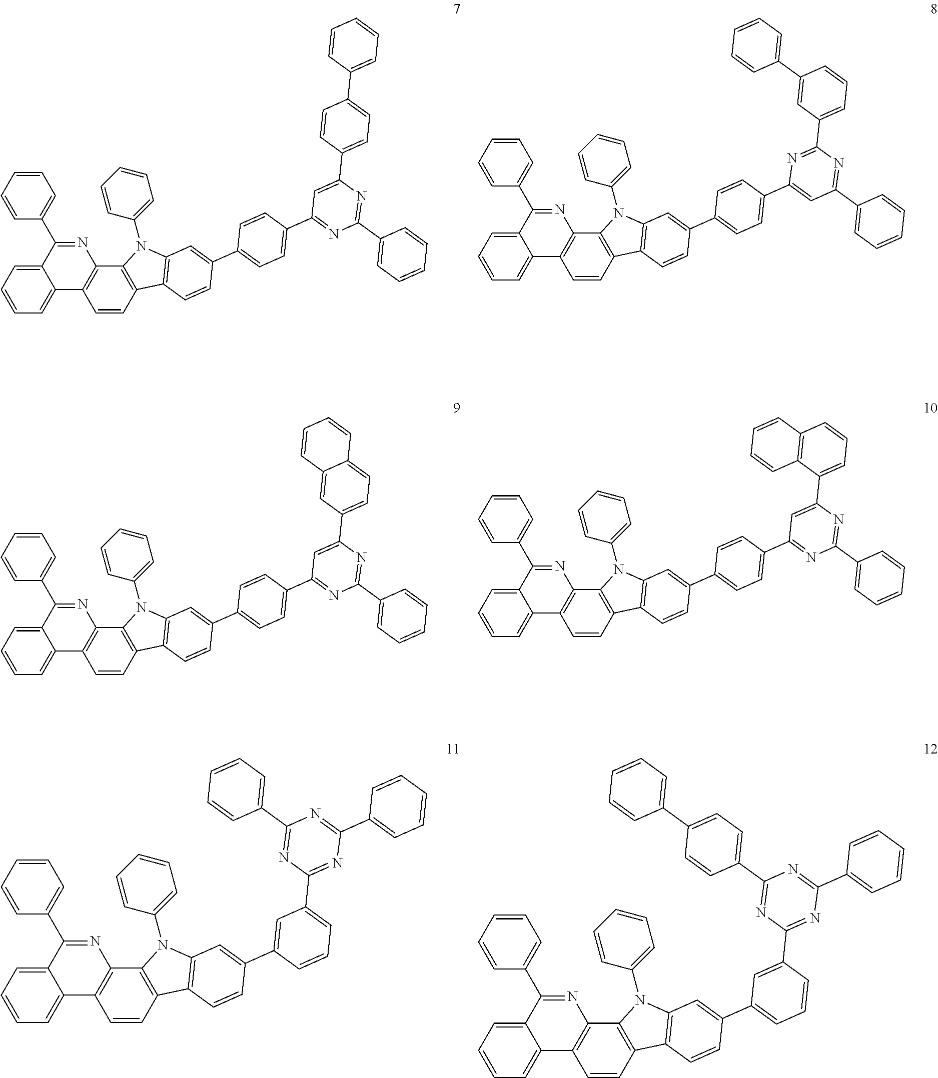

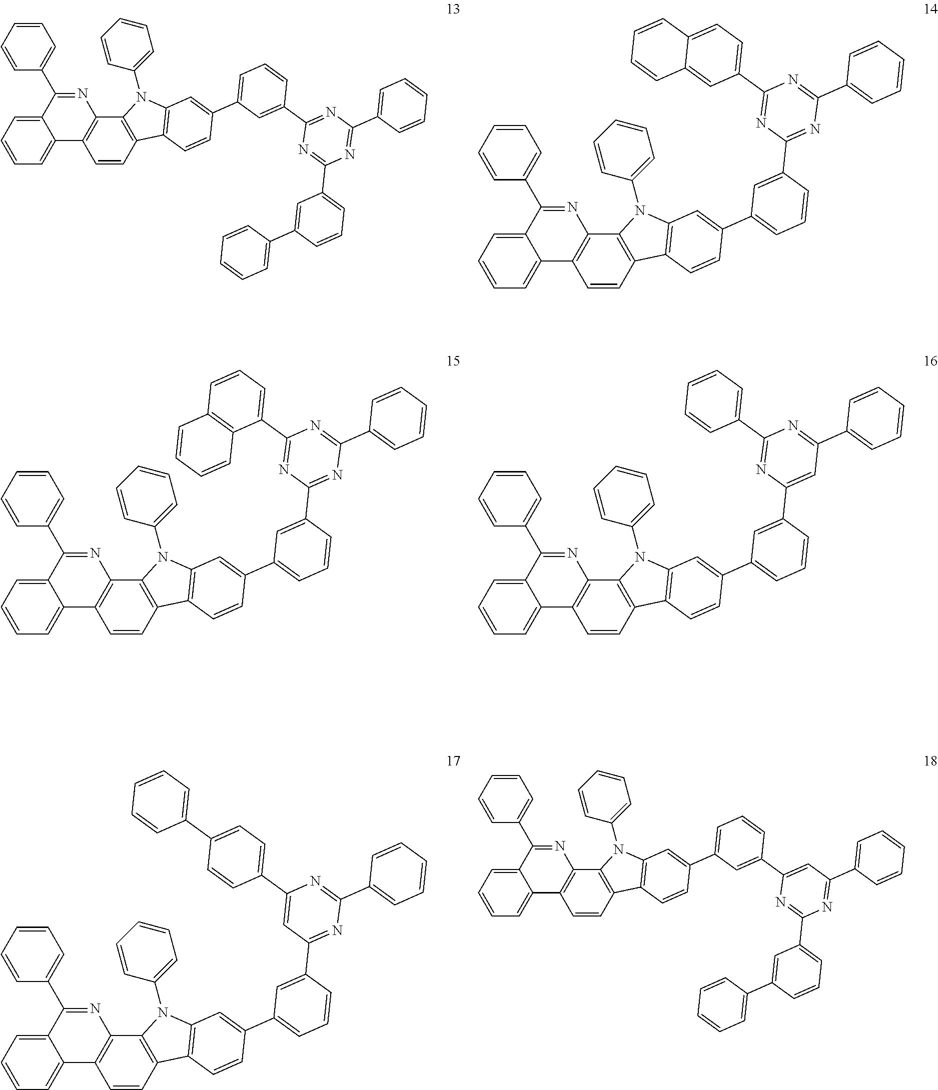

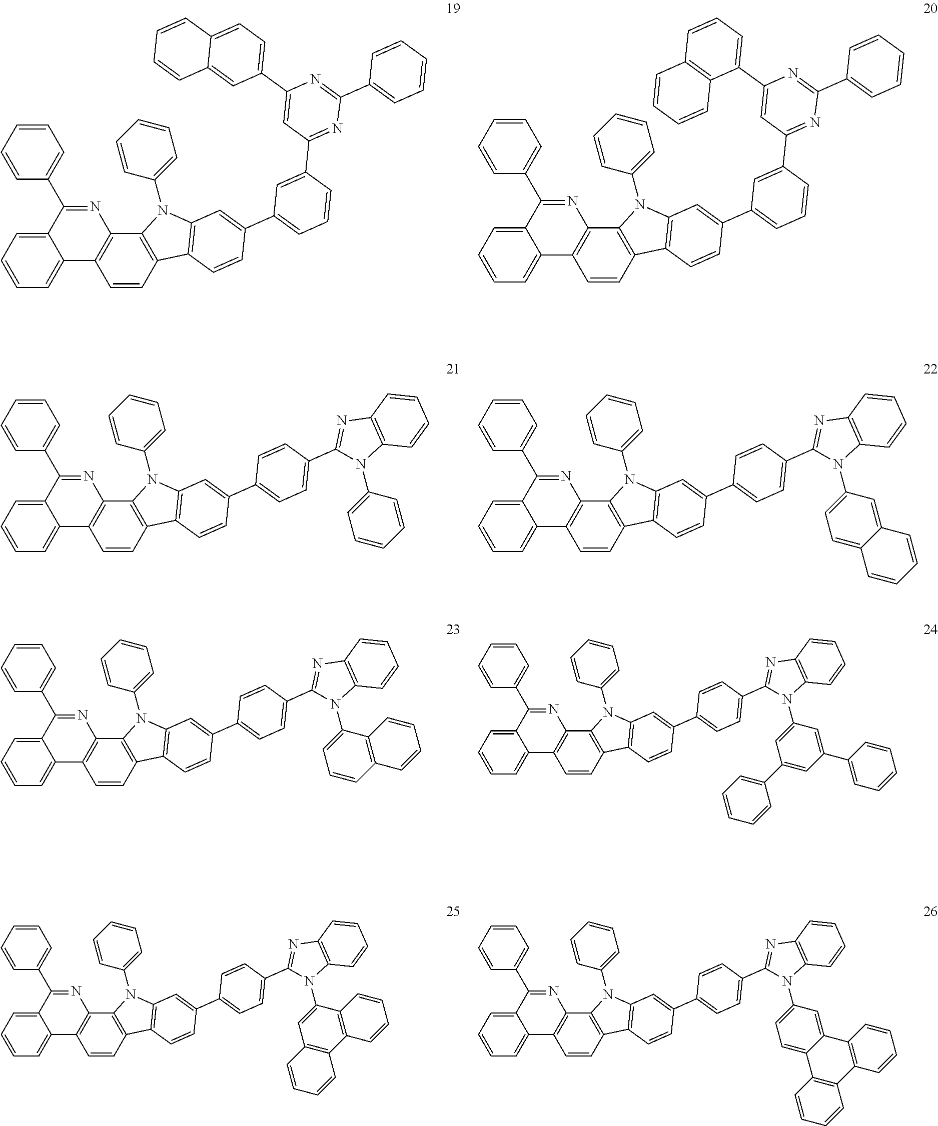

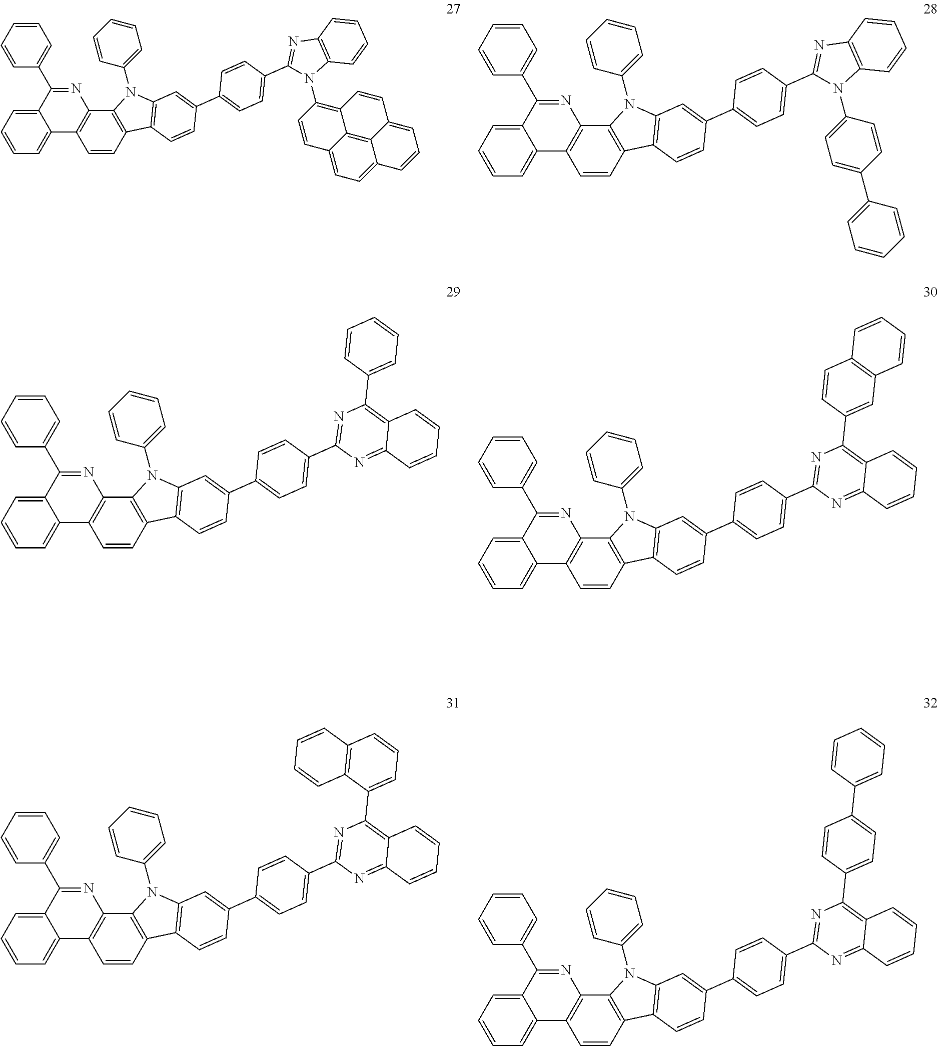

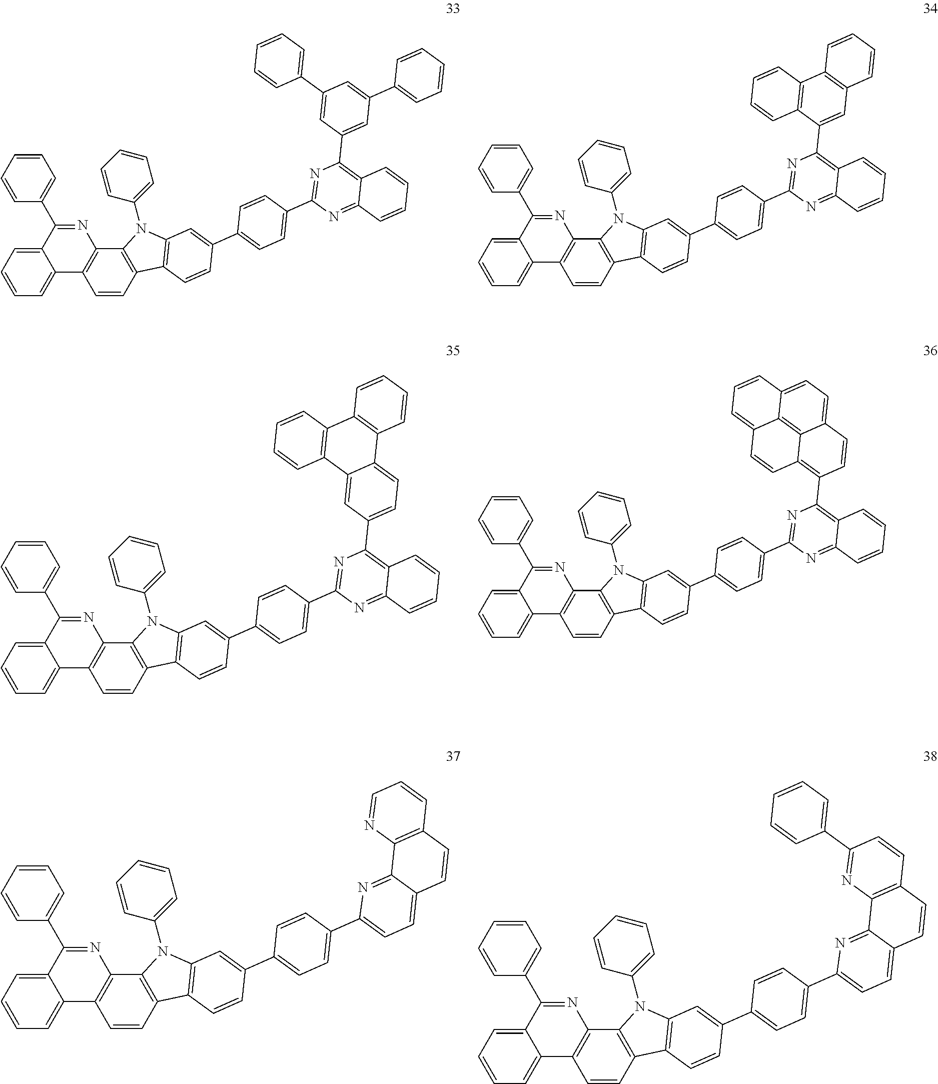

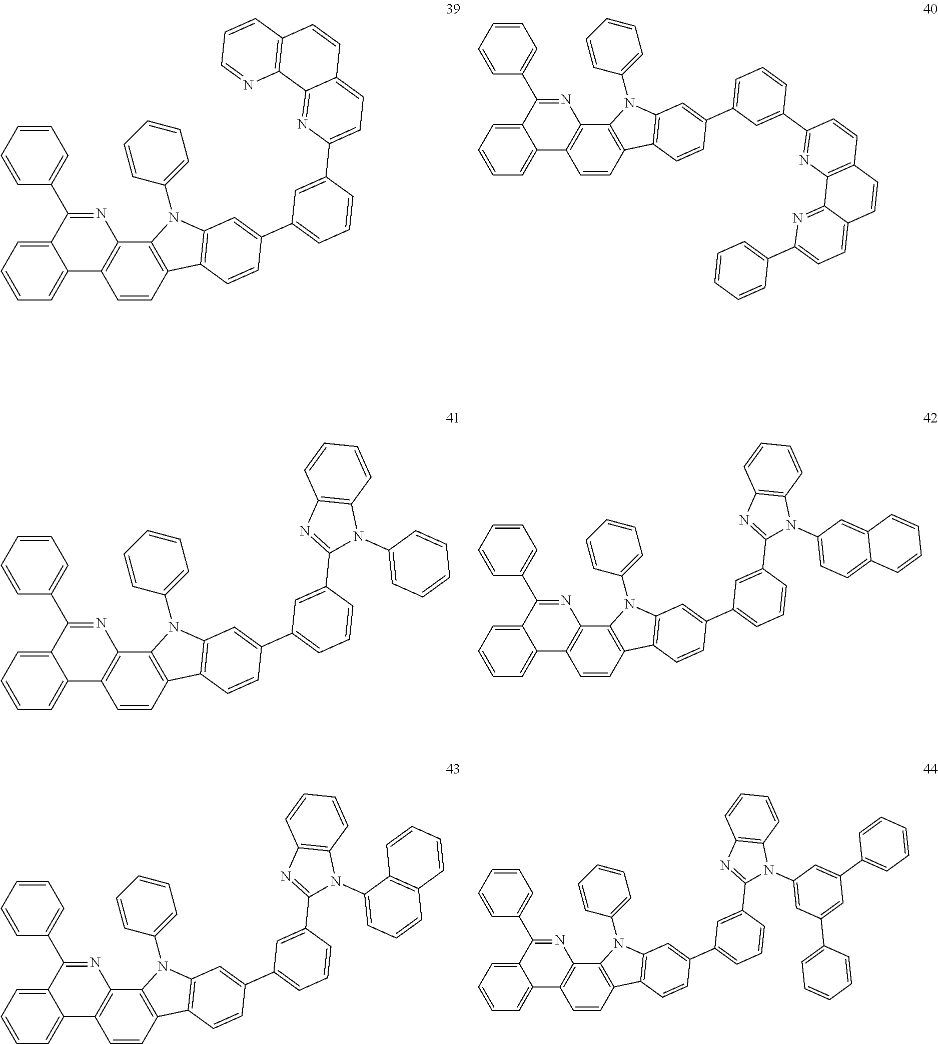

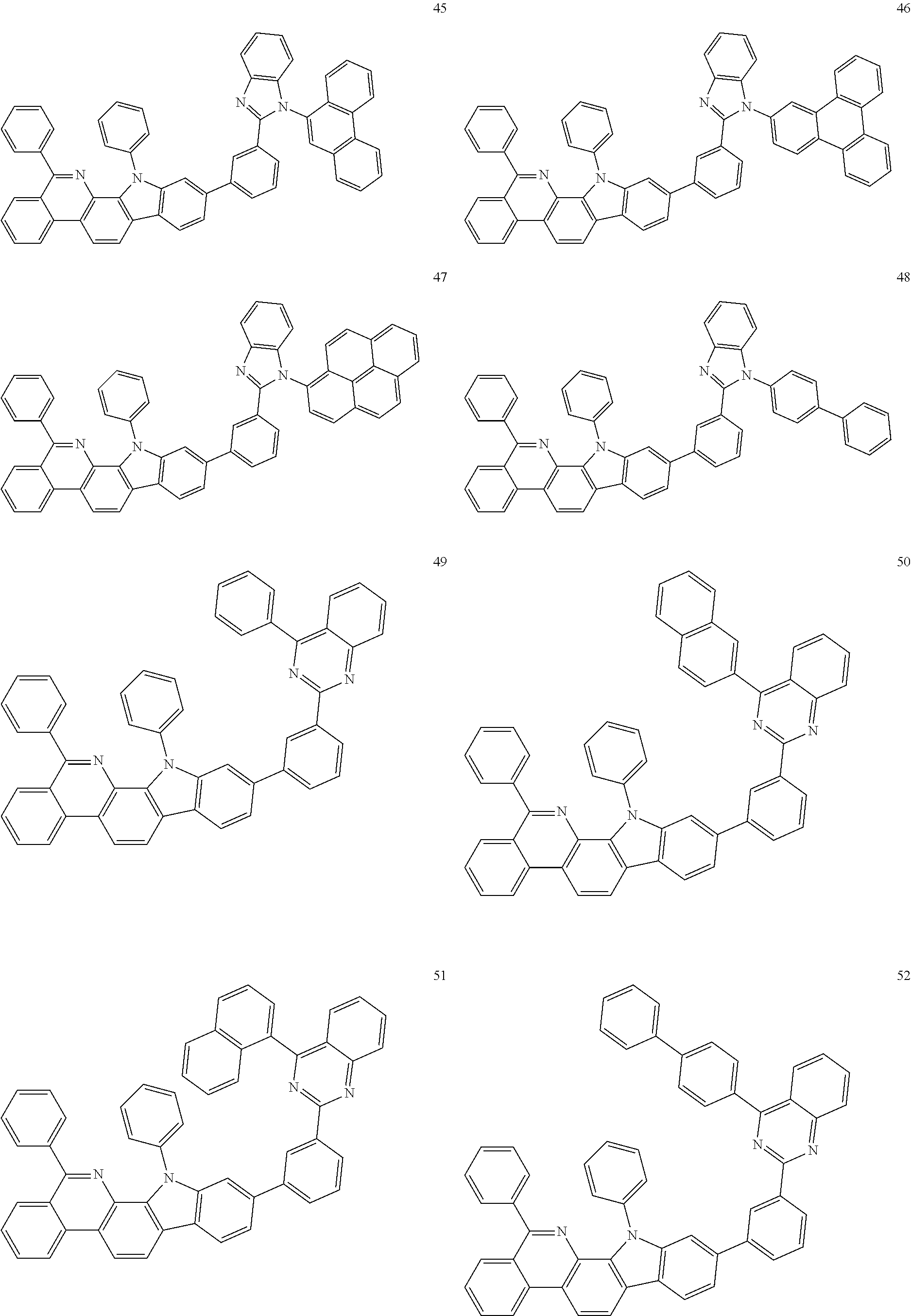

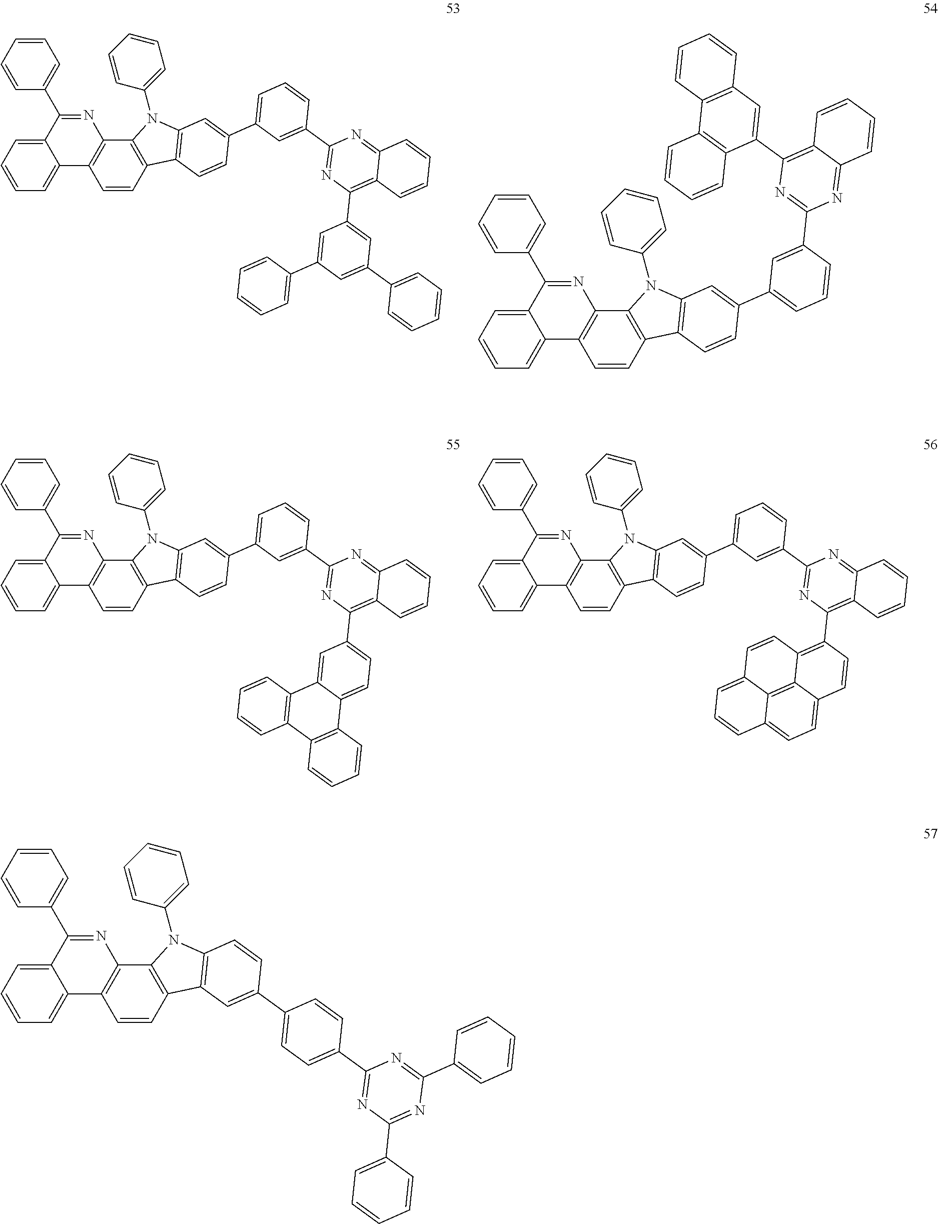

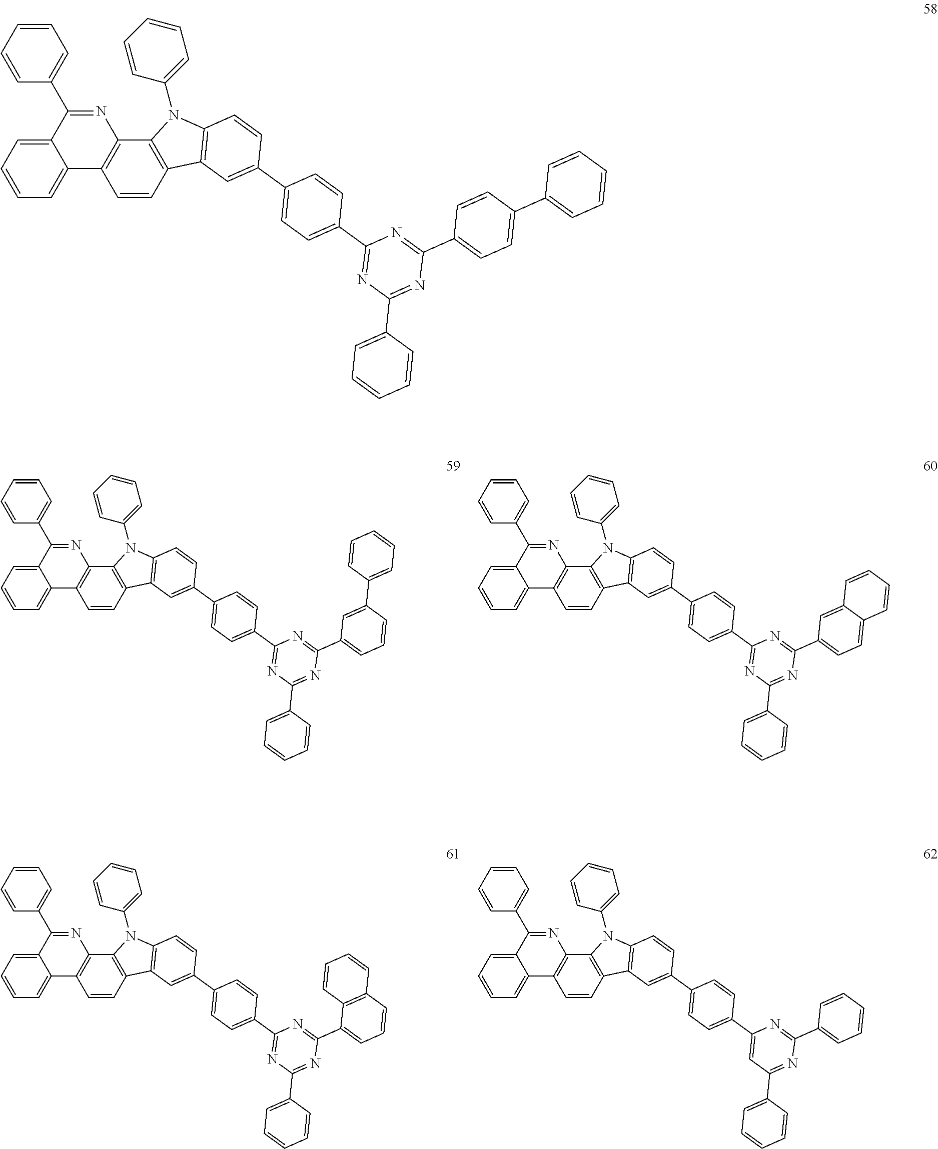

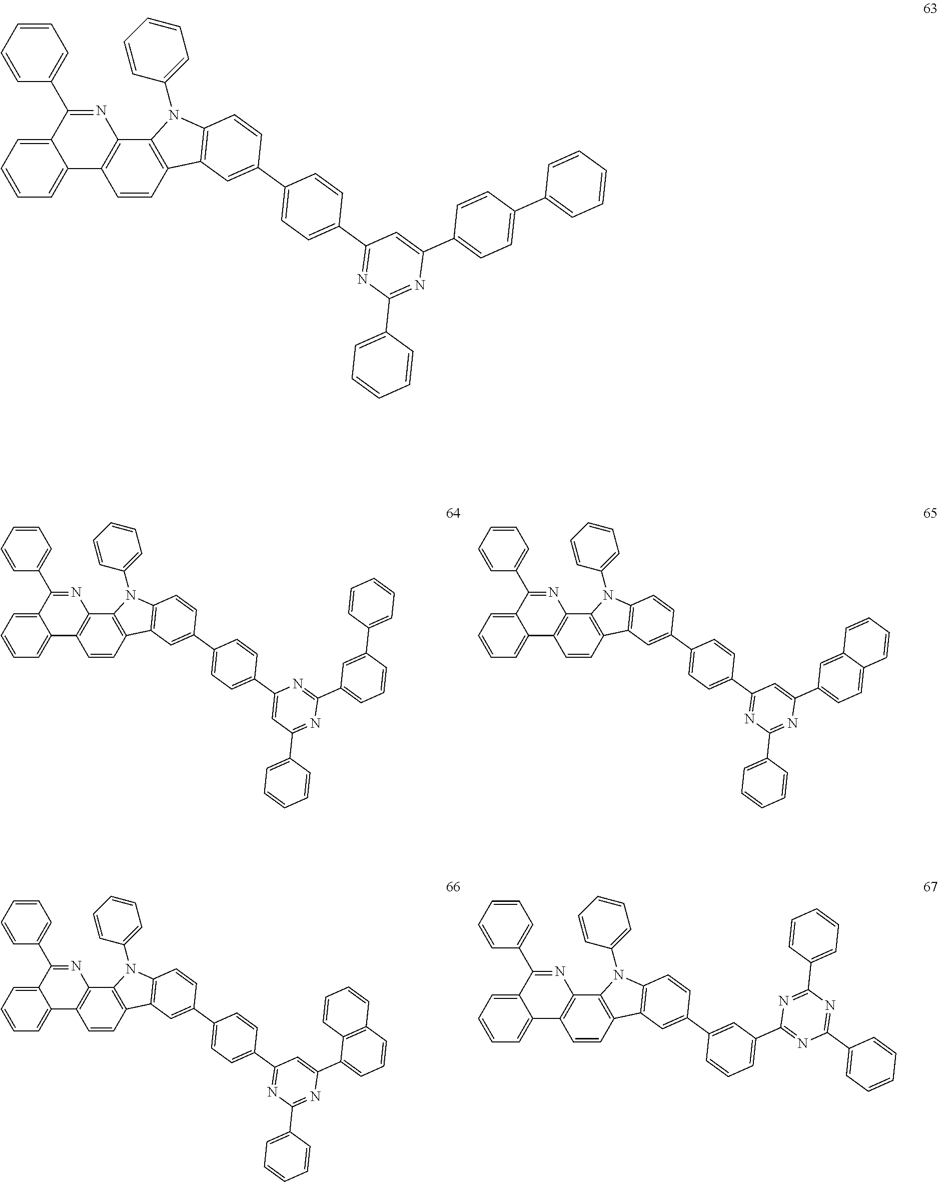

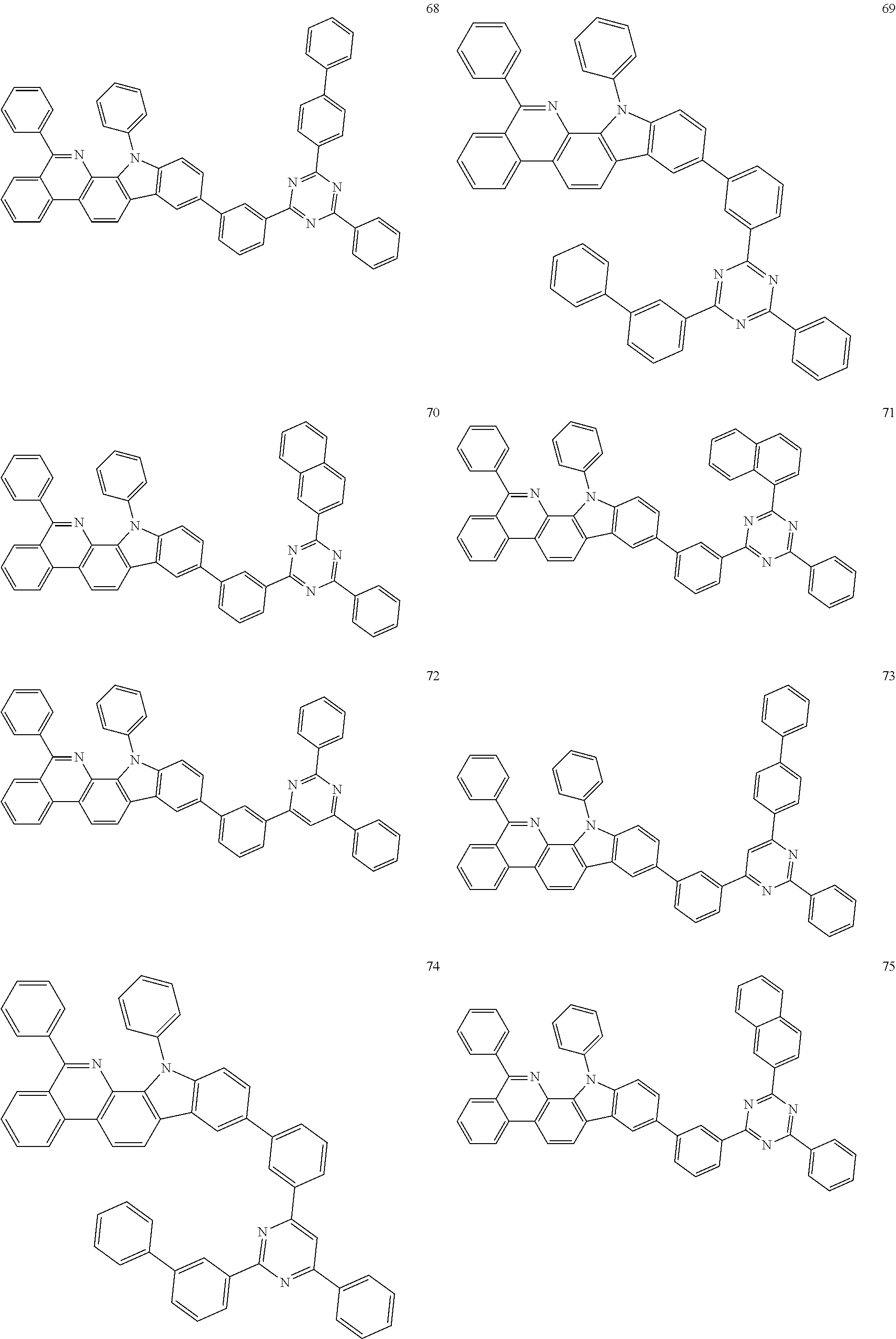

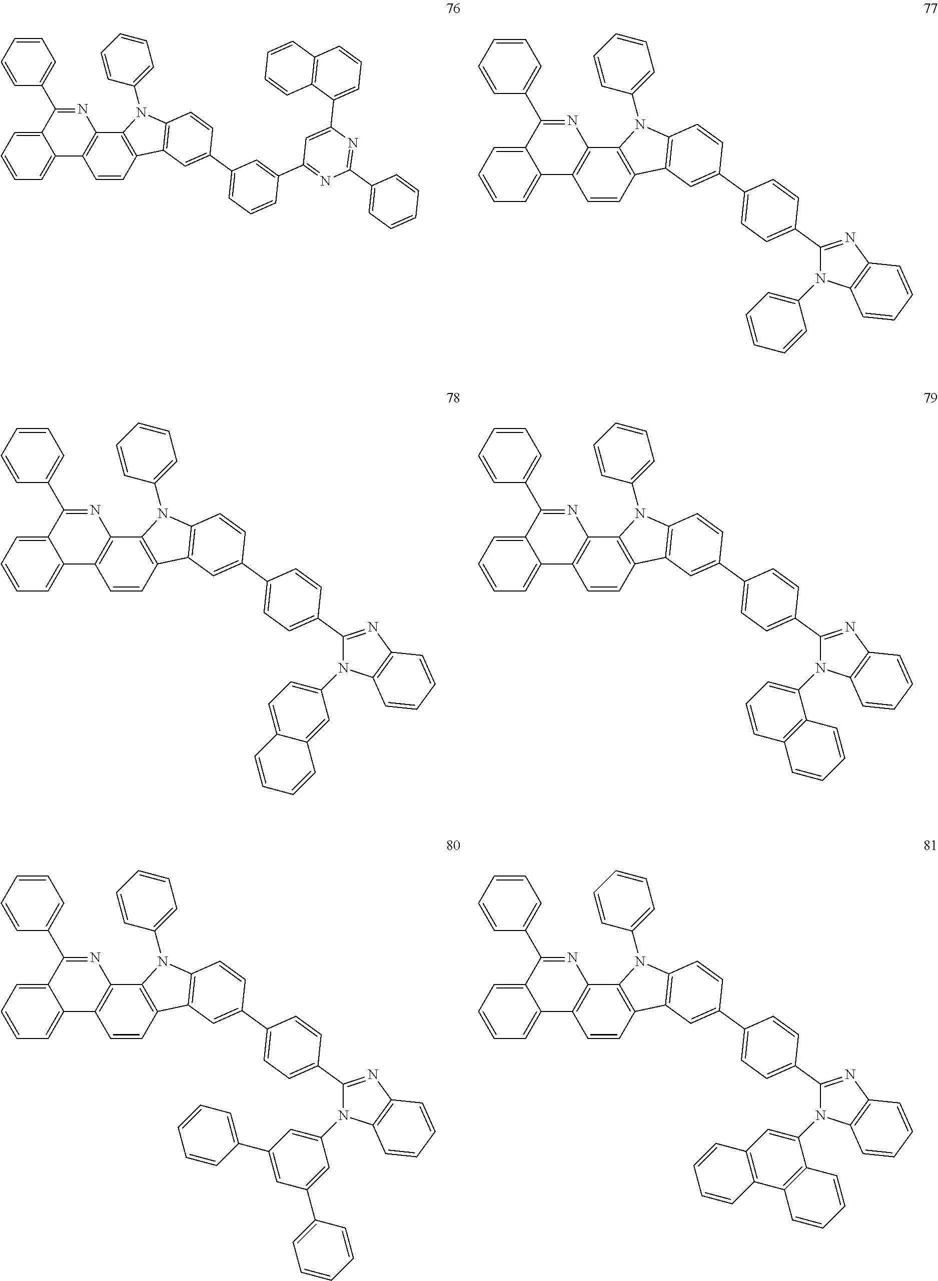

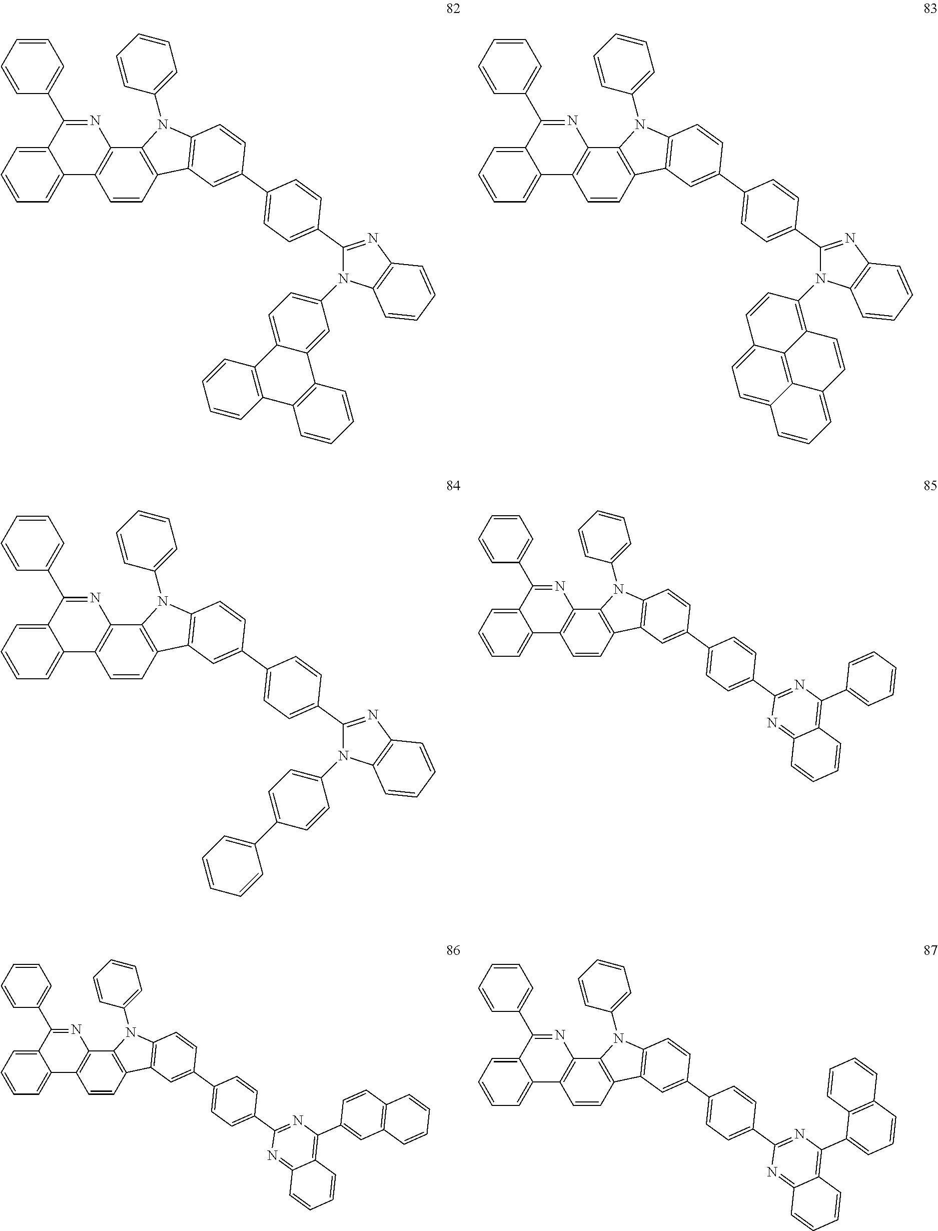

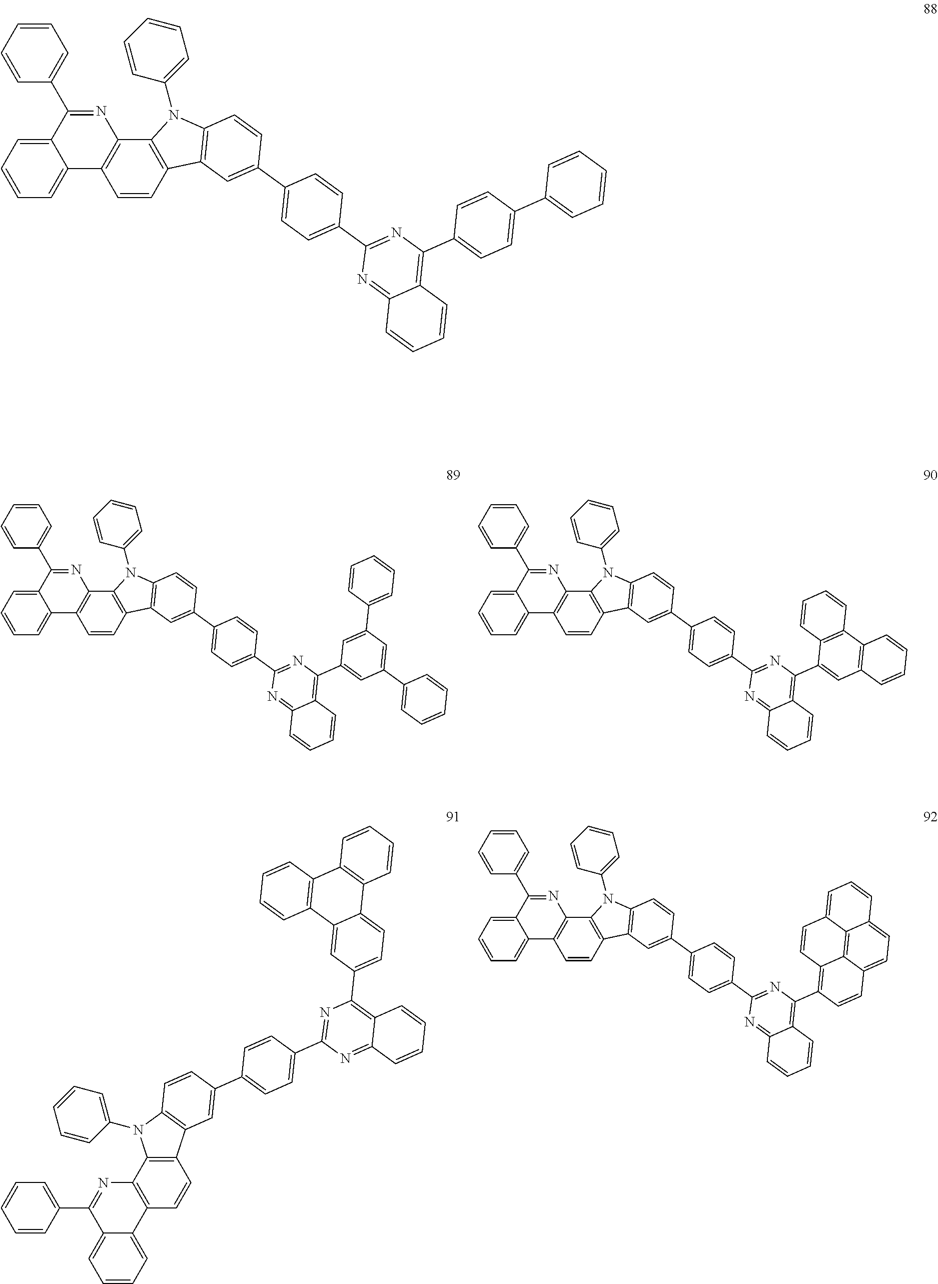

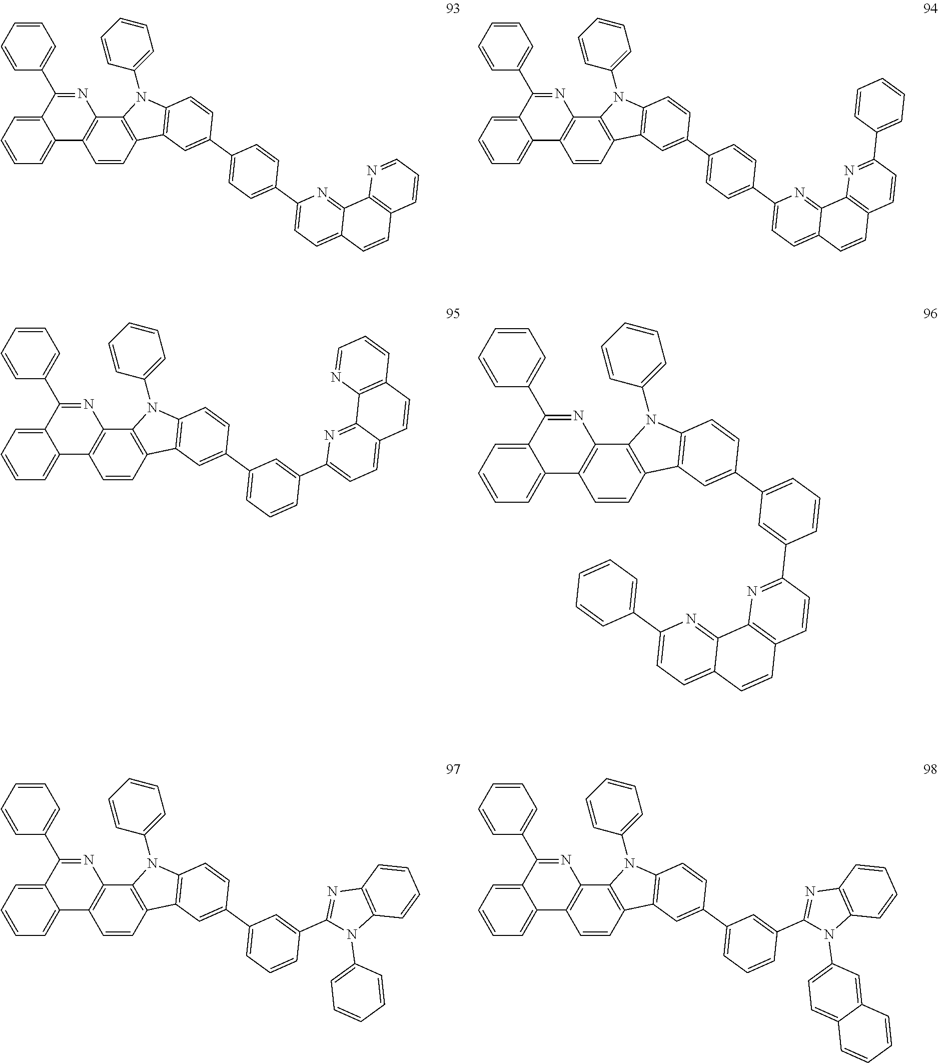

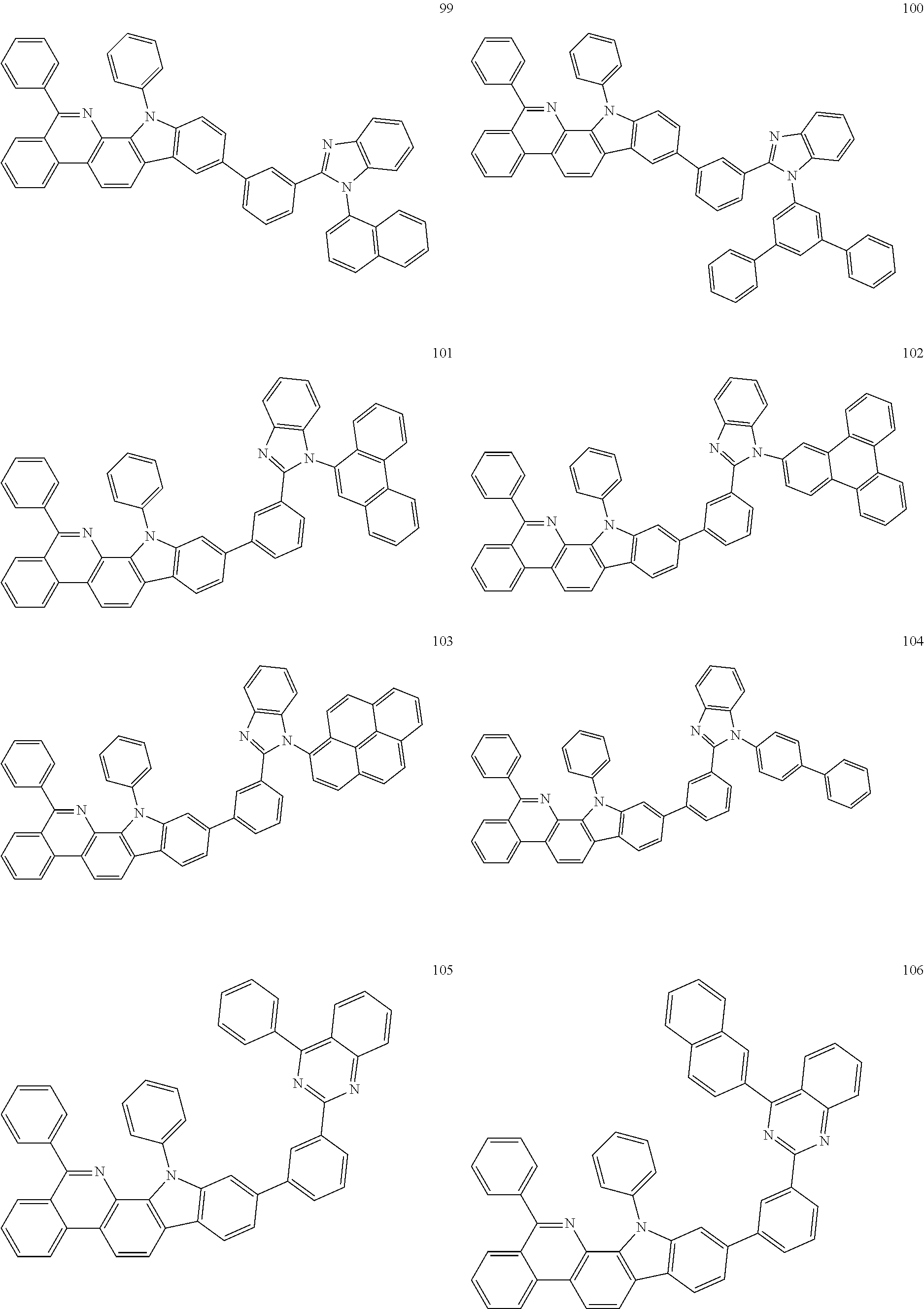

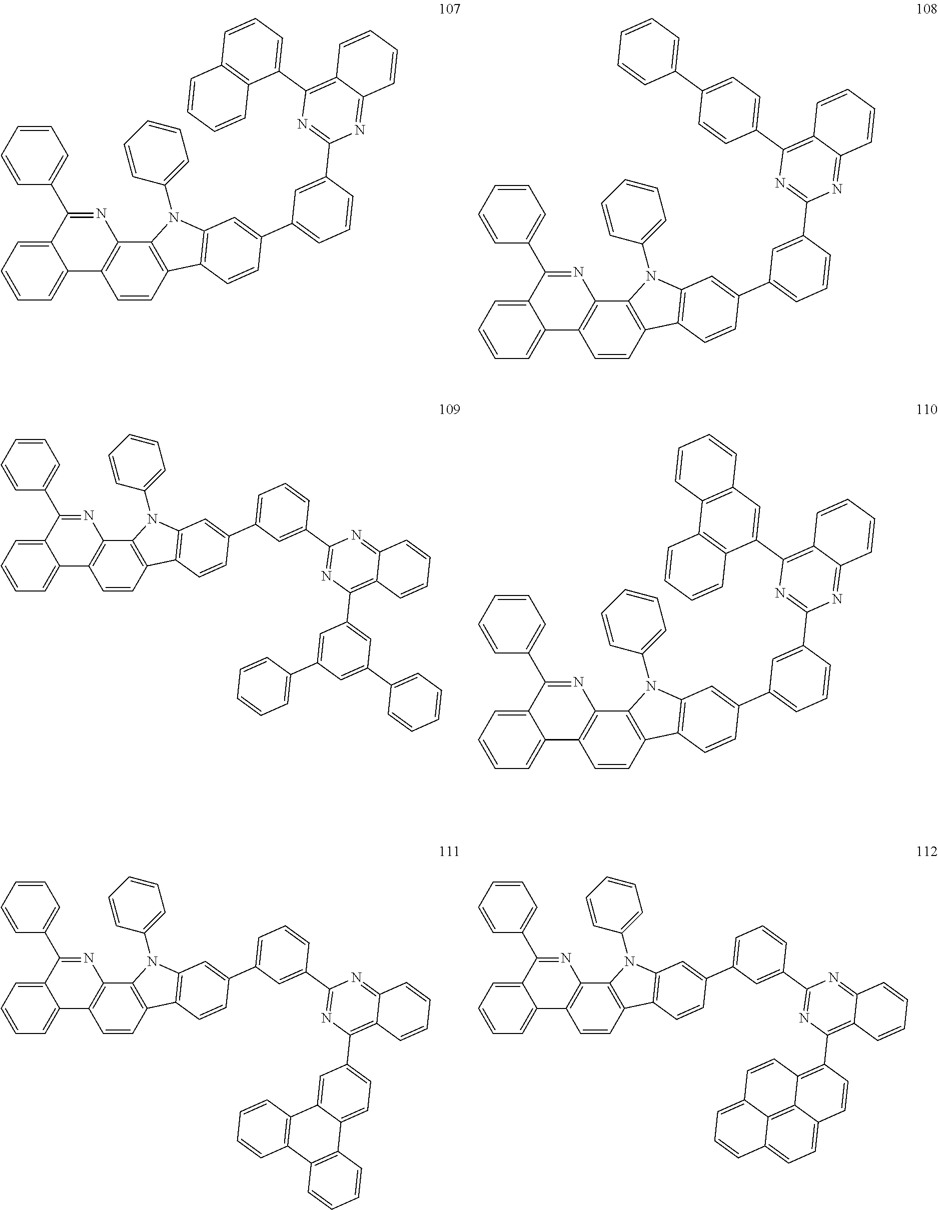

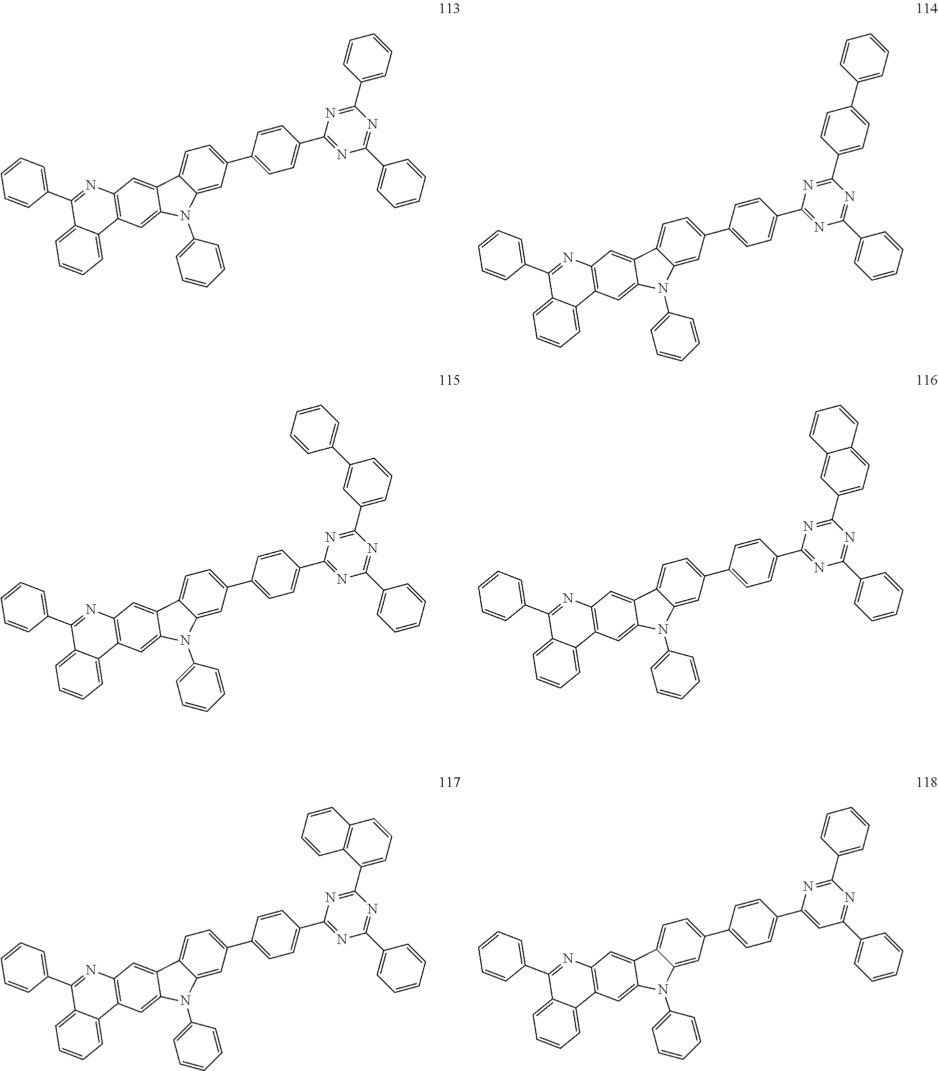

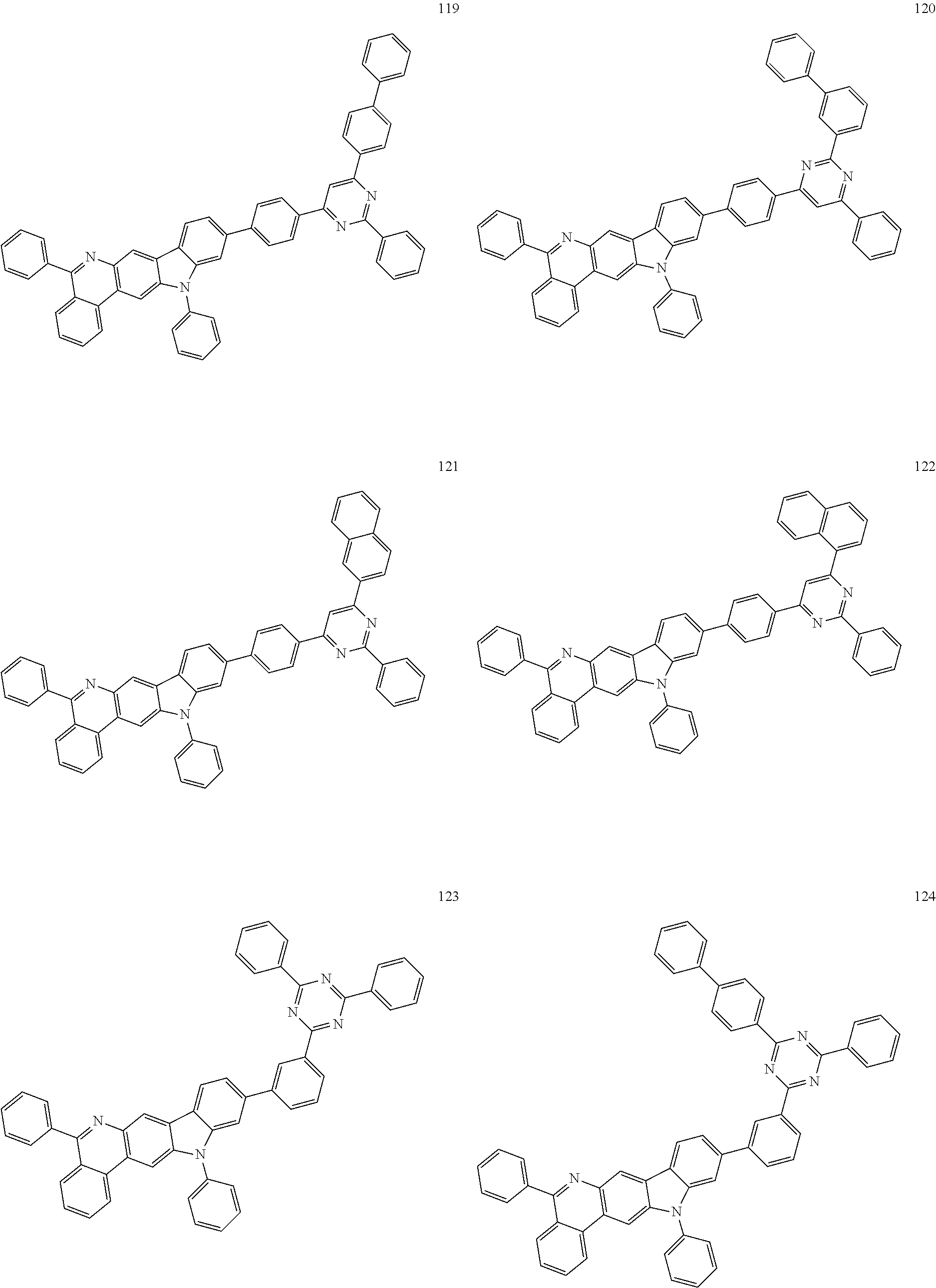

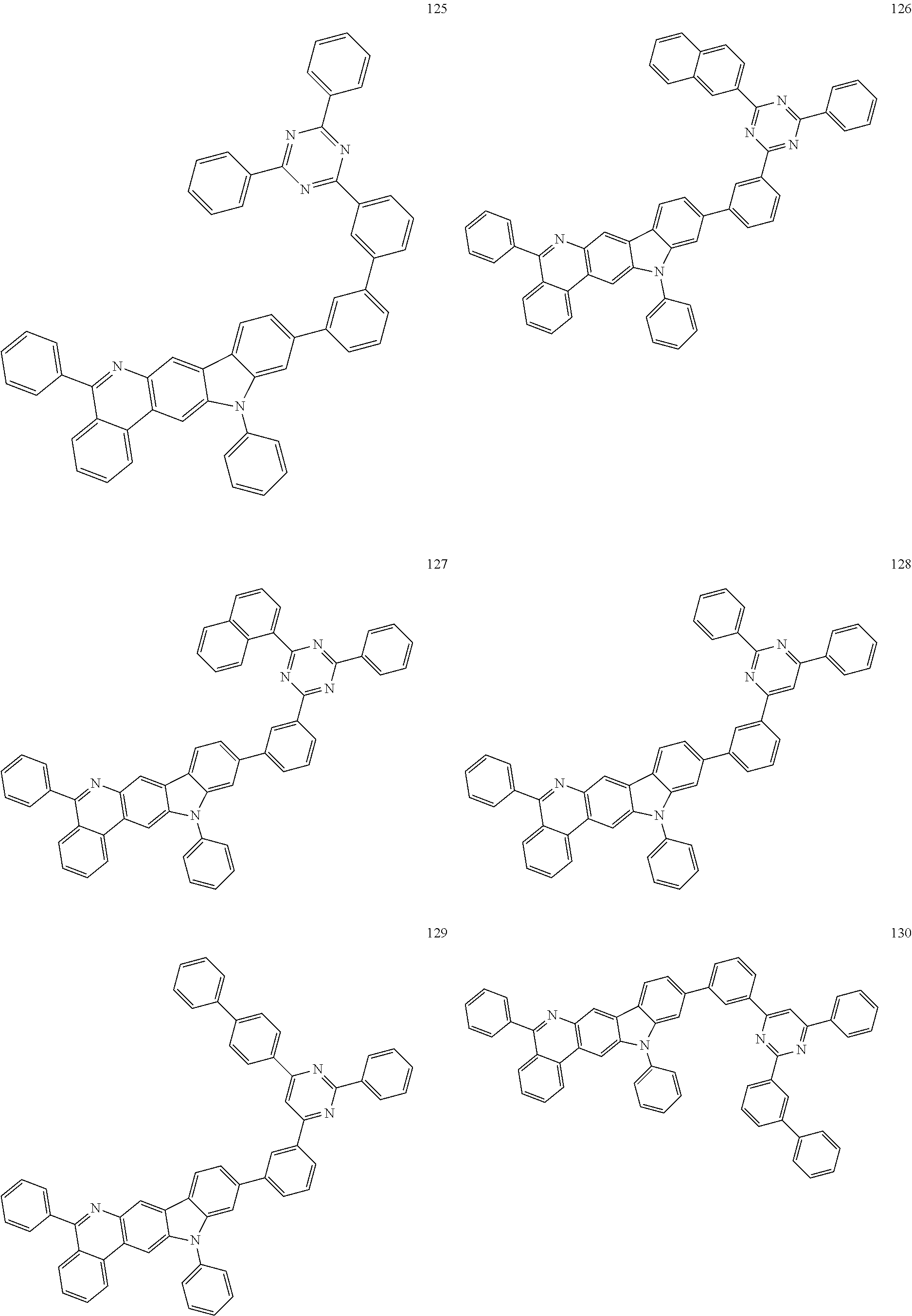

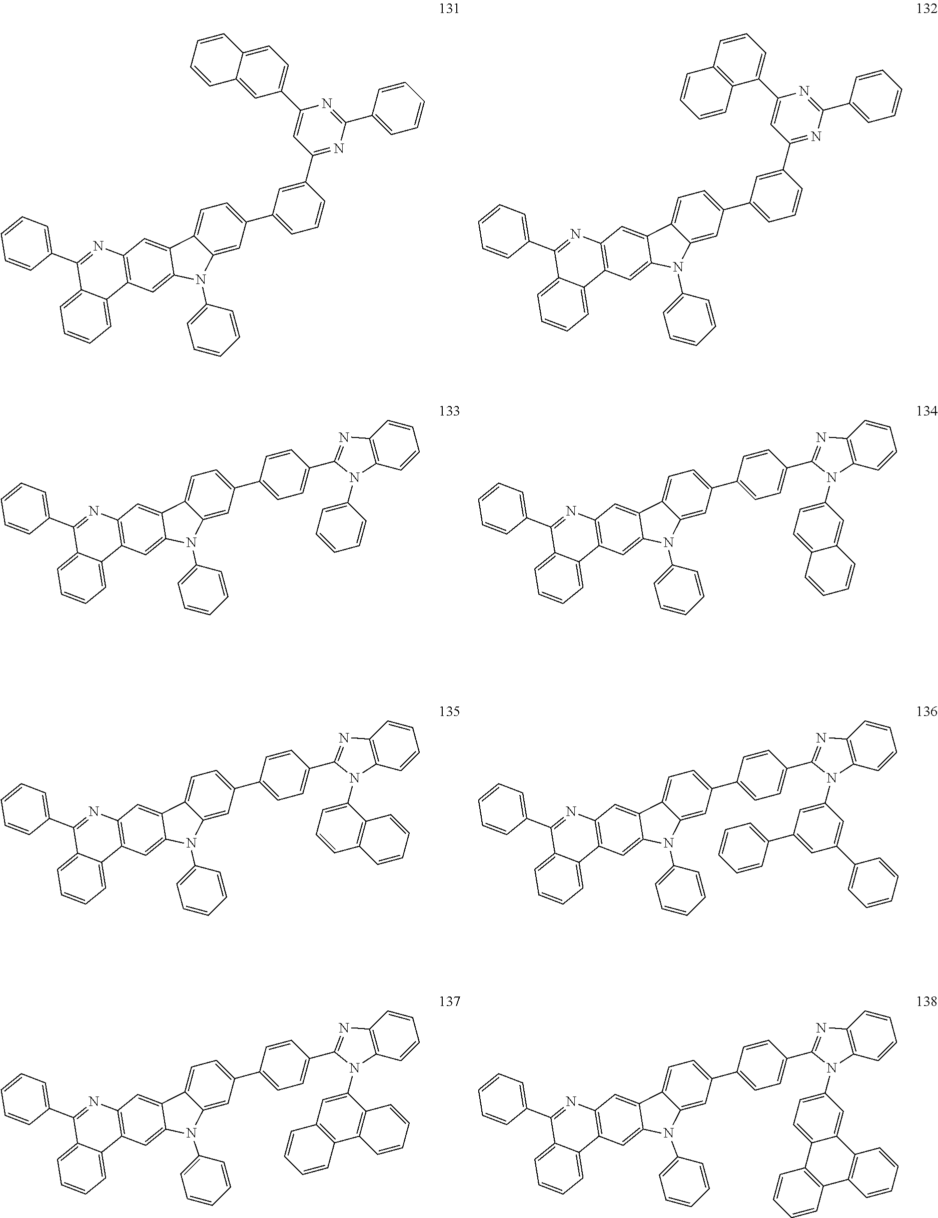

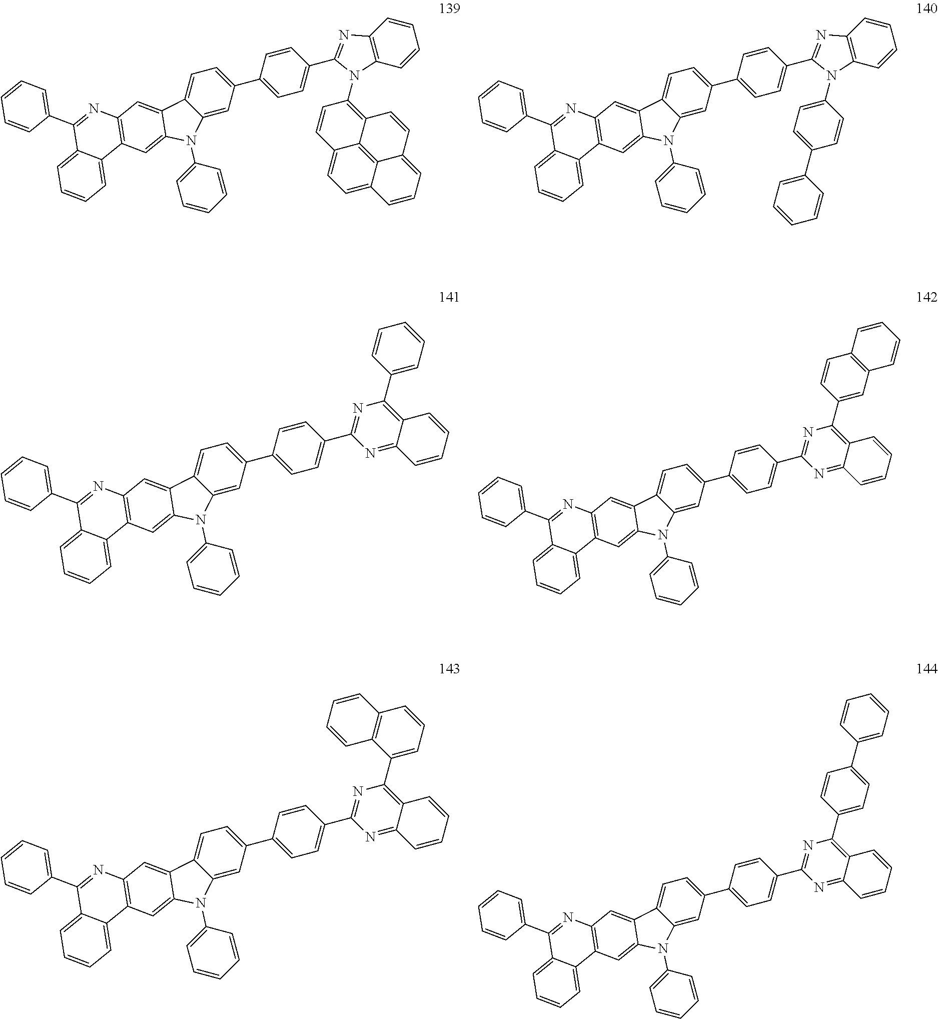

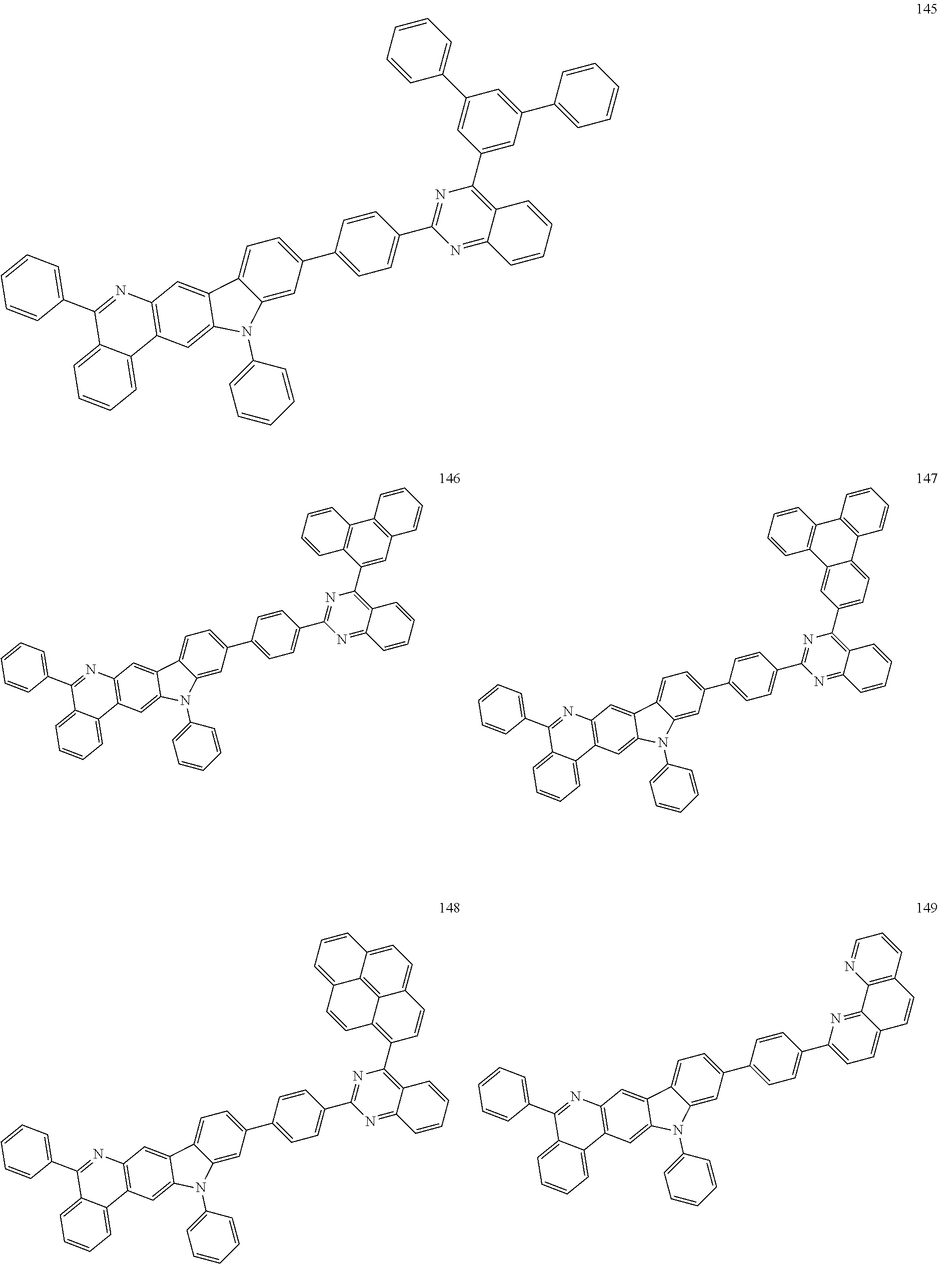

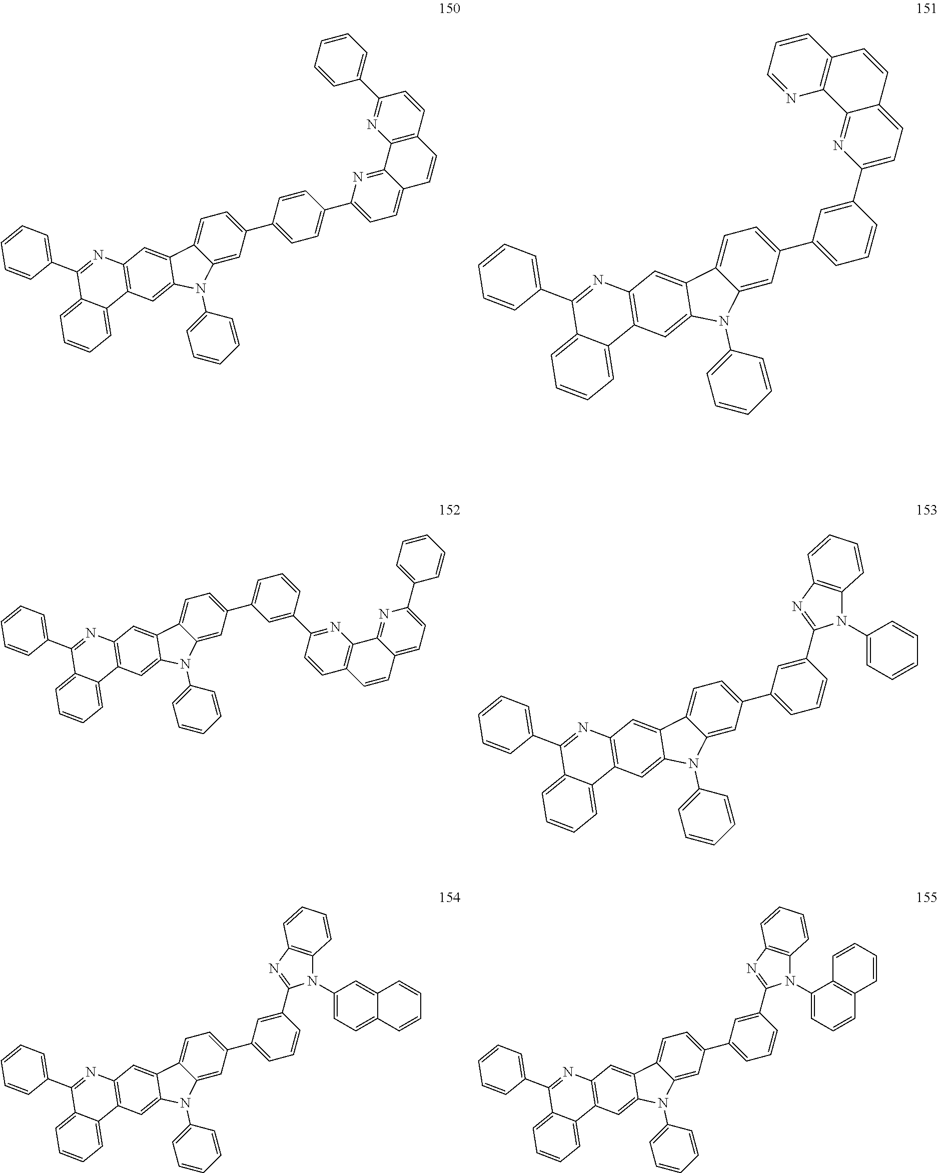

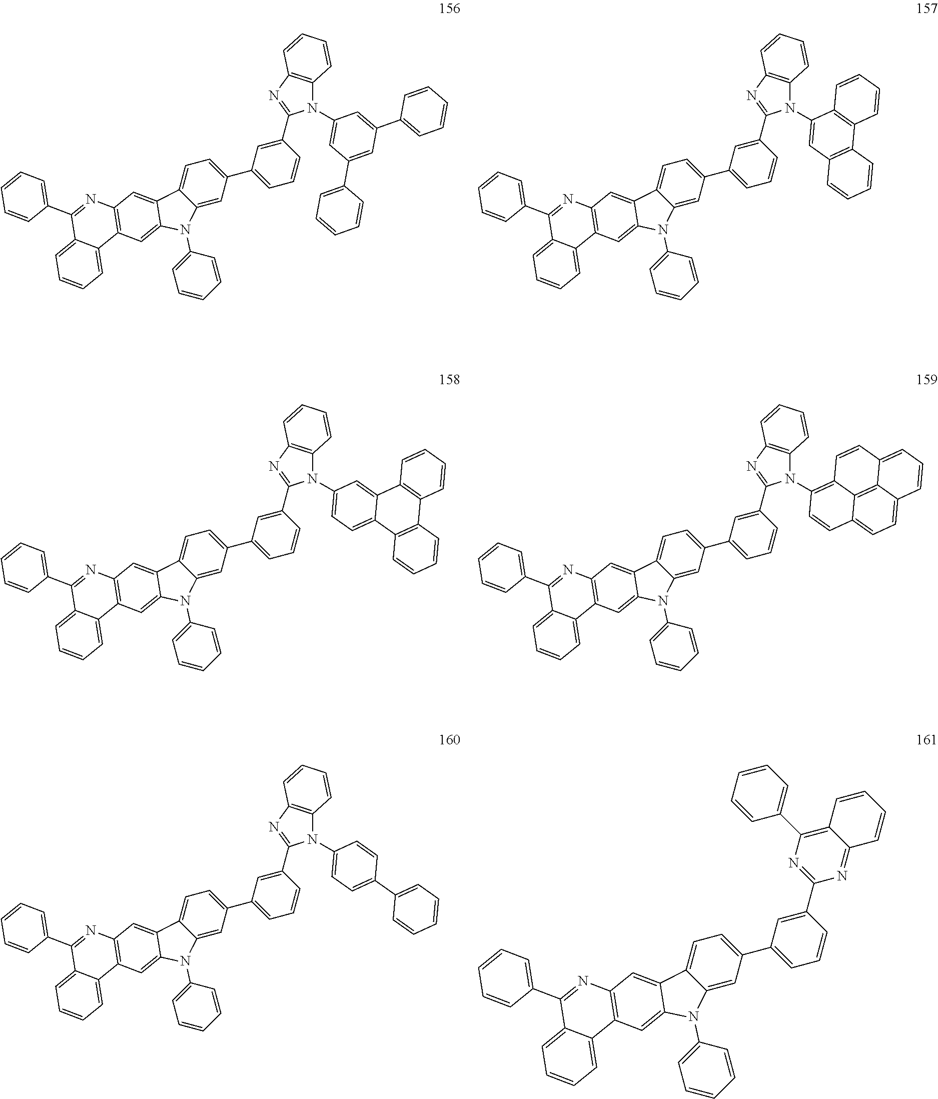

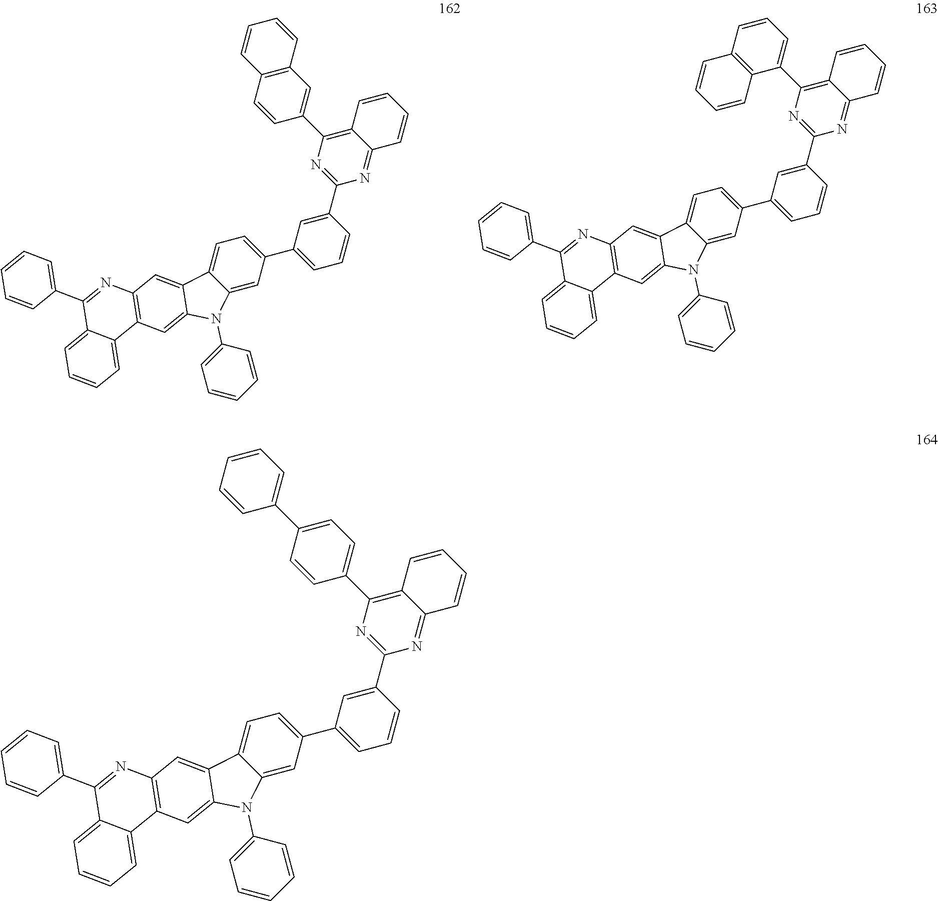 [SMILES: c1ccc(-c2nc3cc4c5ccc(-c6cccc(-c7nc(-c8ccc9ccccc9c8)c8ccccc8n7)c6)cc5n(-c5ccccc5)c4cc3c3ccccc23)cc1 ; c1ccc(-c2nc3cc4c5ccc(-c6cccc(-c7nc(-c8cccc9ccccc89)c8ccccc8n7)c6)cc5n(-c5ccccc5)c4cc3c3ccccc23)cc1 ; c1ccc(-c2ccc(-c3nc(-c4cccc(-c5ccc6c7cc8nc(-c9ccccc9)c9ccccc9c8cc7n(-c7ccccc7)c6c5)c4)nc4ccccc34)cc2)cc1]

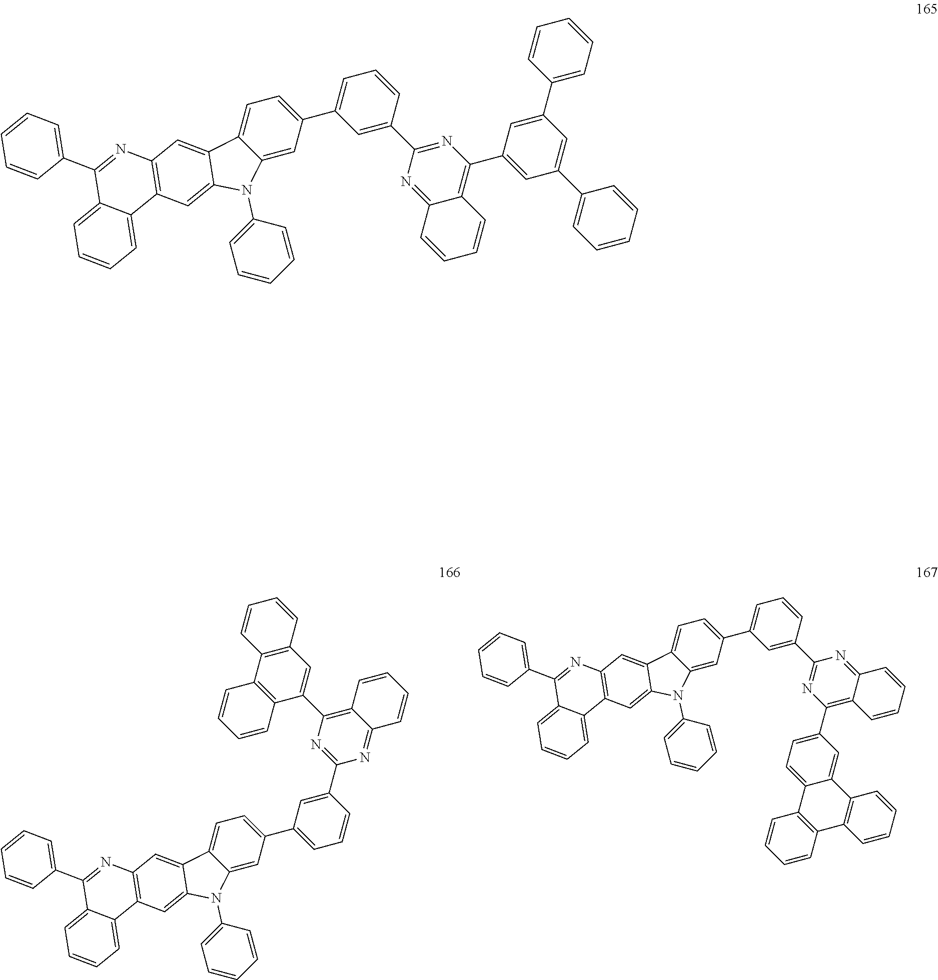 [SMILES: c1ccc(-c2cc(-c3ccccc3)cc(-c3nc(-c4cccc(-c5ccc6c7cc8nc(-c9ccccc9)c9ccccc9c8cc7n(-c7ccccc7)c6c5)c4)nc4ccccc34)c2)cc1 ; c1ccc(-c2nc3cc4c5ccc(-c6cccc(-c7nc(-c8cc9ccccc9c9ccccc89)c8ccccc8n7)c6)cc5n(-c5ccccc5)c4cc3c3ccccc23)cc1 ; c1ccc(-c2nc3cc4c5ccc(-c6cccc(-c7nc(-c8ccc9c%10ccccc%10c%10ccccc%10c9c8)c8ccccc8n7)c6)cc5n(-c5ccccc5)c4cc3c3ccccc23)cc1]

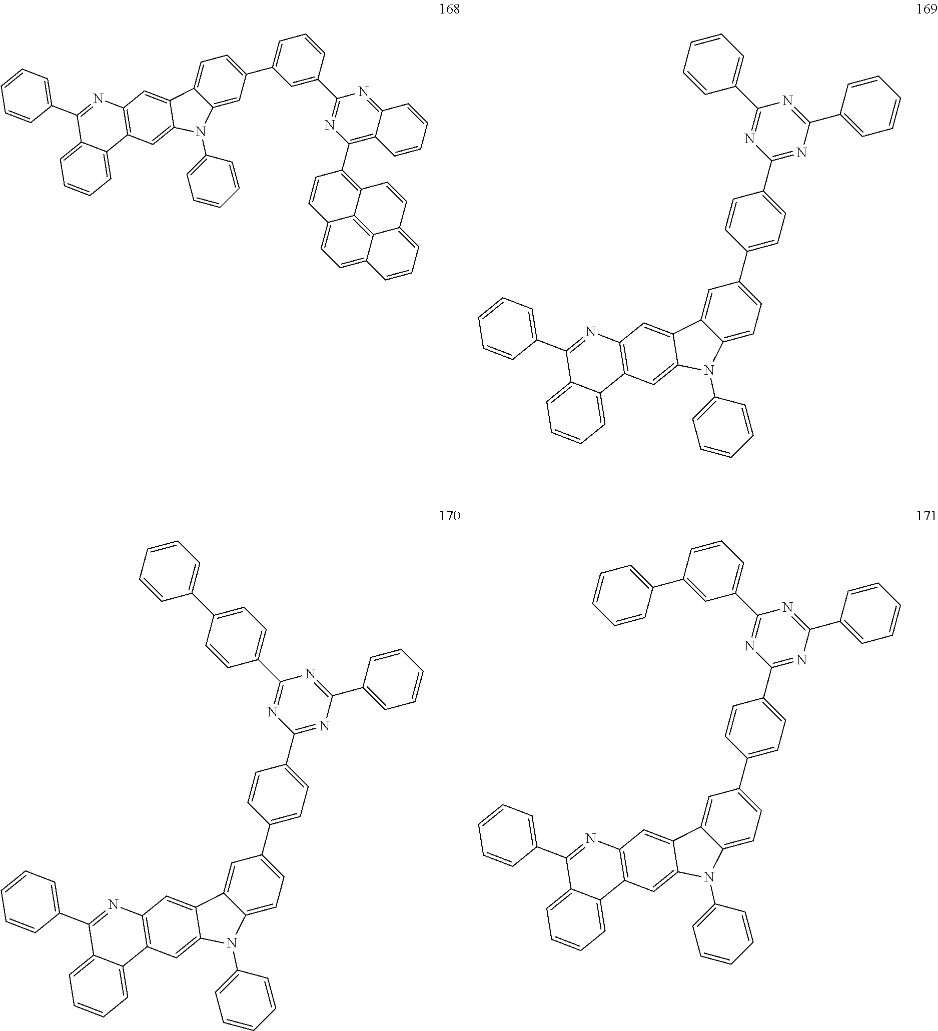 [SMILES: c1ccc(-c2nc3cc4c5ccc(-c6cccc(-c7nc(-c8ccc9ccc%10cccc%11ccc8c9c%10%11)c8ccccc8n7)c6)cc5n(-c5ccccc5)c4cc3c3ccccc23)cc1 ; c1ccc(-c2nc(-c3ccccc3)nc(-c3ccc(-c4ccc5c(c4)c4cc6nc(-c7ccccc7)c7ccccc7c6cc4n5-c4ccccc4)cc3)n2)cc1 ; c1ccc(-c2ccc(-c3nc(-c4ccccc4)nc(-c4ccc(-c5ccc6c(c5)c5cc7nc(-c8ccccc8)c8ccccc8c7cc5n6-c5ccccc5)cc4)n3)cc2)cc1 ; c1ccc(-c2cccc(-c3nc(-c4ccccc4)nc(-c4ccc(-c5ccc6c(c5)c5cc7nc(-c8ccccc8)c8ccccc8c7cc5n6-c5ccccc5)cc4)n3)c2)cc1]

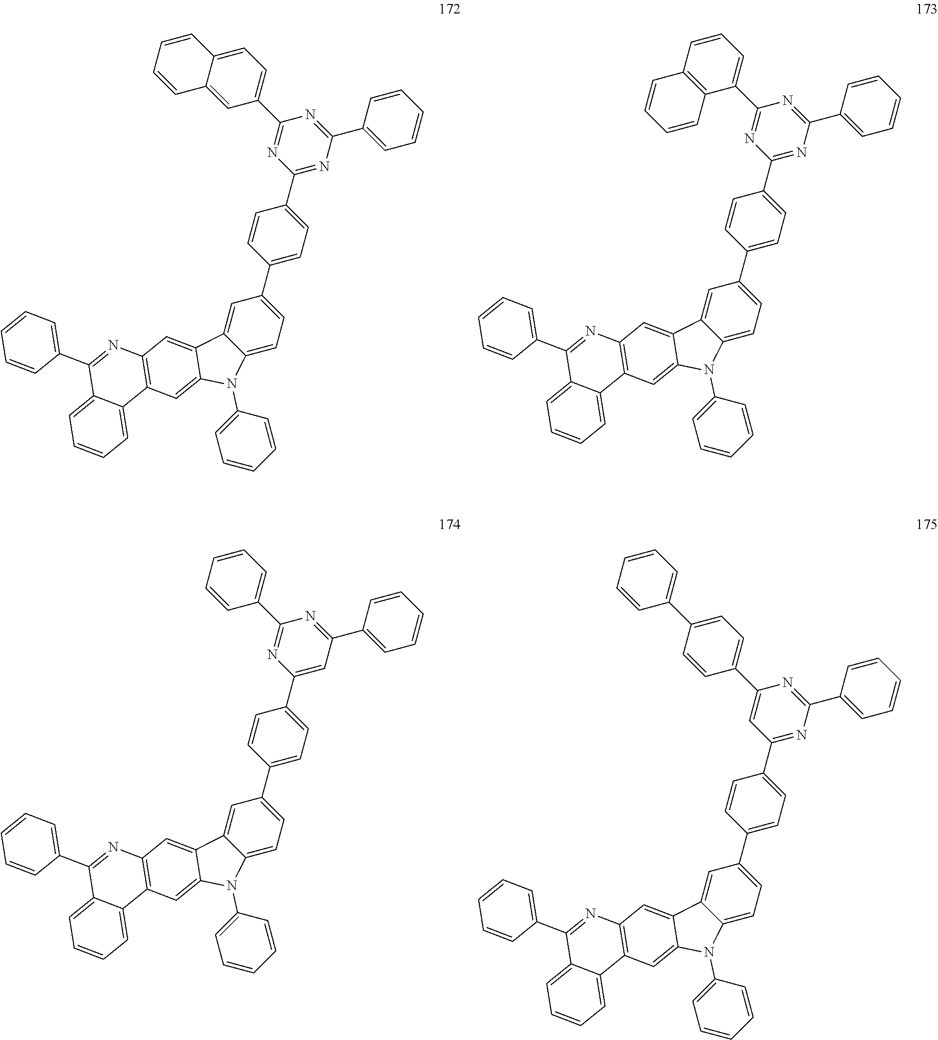 [SMILES: c1ccc(-c2nc(-c3ccc(-c4ccc5c(c4)c4cc6nc(-c7ccccc7)c7ccccc7c6cc4n5-c4ccccc4)cc3)nc(-c3ccc4ccccc4c3)n2)cc1 ; c1ccc(-c2nc(-c3ccc(-c4ccc5c(c4)c4cc6nc(-c7ccccc7)c7ccccc7c6cc4n5-c4ccccc4)cc3)nc(-c3cccc4ccccc34)n2)cc1 ; c1ccc(-c2cc(-c3ccc(-c4ccc5c(c4)c4cc6nc(-c7ccccc7)c7ccccc7c6cc4n5-c4ccccc4)cc3)nc(-c3ccccc3)n2)cc1 ; c1ccc(-c2ccc(-c3cc(-c4ccc(-c5ccc6c(c5)c5cc7nc(-c8ccccc8)c8ccccc8c7cc5n6-c5ccccc5)cc4)nc(-c4ccccc4)n3)cc2)cc1]

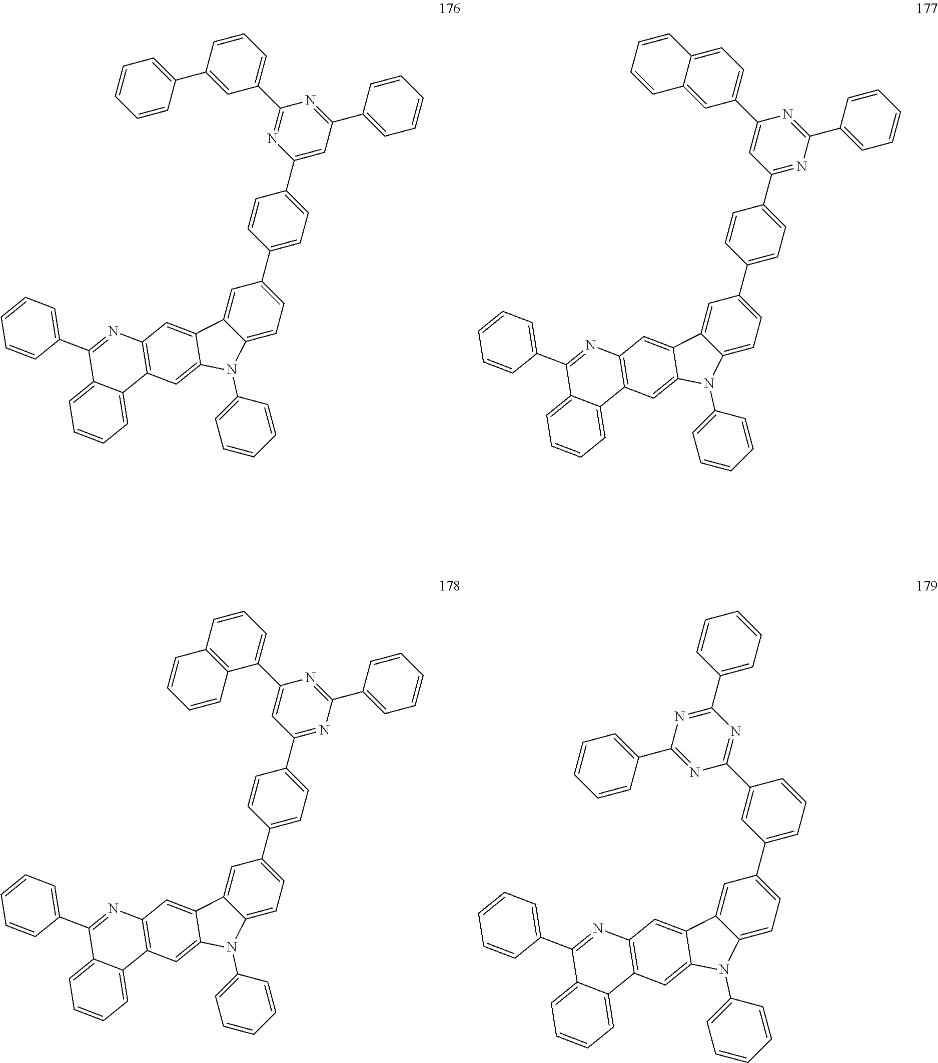 [SMILES: c1ccc(-c2cccc(-c3nc(-c4ccccc4)cc(-c4ccc(-c5ccc6c(c5)c5cc7nc(-c8ccccc8)c8ccccc8c7cc5n6-c5ccccc5)cc4)n3)c2)cc1 ; c1ccc(-c2nc(-c3ccc(-c4ccc5c(c4)c4cc6nc(-c7ccccc7)c7ccccc7c6cc4n5-c4ccccc4)cc3)cc(-c3ccc4ccccc4c3)n2)cc1 ; c1ccc(-c2nc(-c3ccc(-c4ccc5c(c4)c4cc6nc(-c7ccccc7)c7ccccc7c6cc4n5-c4ccccc4)cc3)cc(-c3cccc4ccccc34)n2)cc1 ; c1ccc(-c2nc(-c3ccccc3)nc(-c3cccc(-c4ccc5c(c4)c4cc6nc(-c7ccccc7)c7ccccc7c6cc4n5-c4ccccc4)c3)n2)cc1]

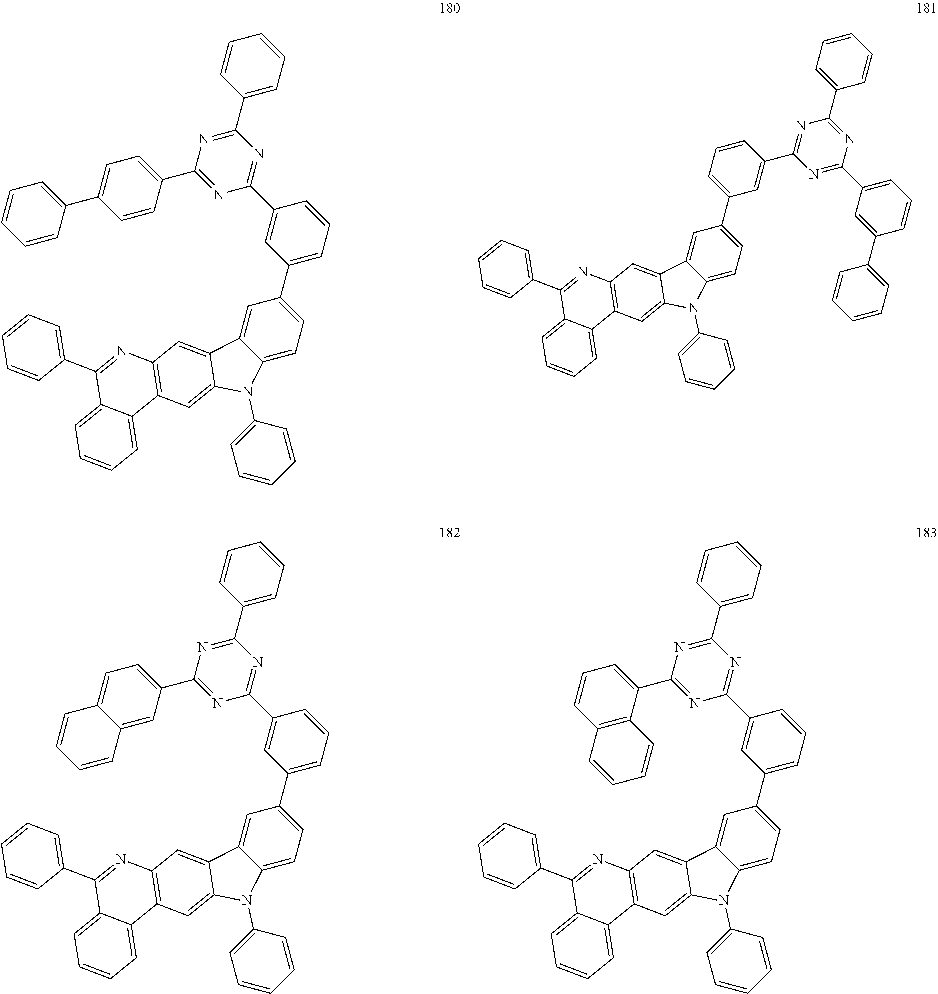 [SMILES: c1ccc(-c2ccc(-c3nc(-c4ccccc4)nc(-c4cccc(-c5ccc6c(c5)c5cc7nc(-c8ccccc8)c8ccccc8c7cc5n6-c5ccccc5)c4)n3)cc2)cc1 ; c1ccc(-c2cccc(-c3nc(-c4ccccc4)nc(-c4cccc(-c5ccc6c(c5)c5cc7nc(-c8ccccc8)c8ccccc8c7cc5n6-c5ccccc5)c4)n3)c2)cc1 ; c1ccc(-c2nc(-c3cccc(-c4ccc5c(c4)c4cc6nc(-c7ccccc7)c7ccccc7c6cc4n5-c4ccccc4)c3)nc(-c3ccc4ccccc4c3)n2)cc1 ; c1ccc(-c2nc(-c3cccc(-c4ccc5c(c4)c4cc6nc(-c7ccccc7)c7ccccc7c6cc4n5-c4ccccc4)c3)nc(-c3cccc4ccccc34)n2)cc1]

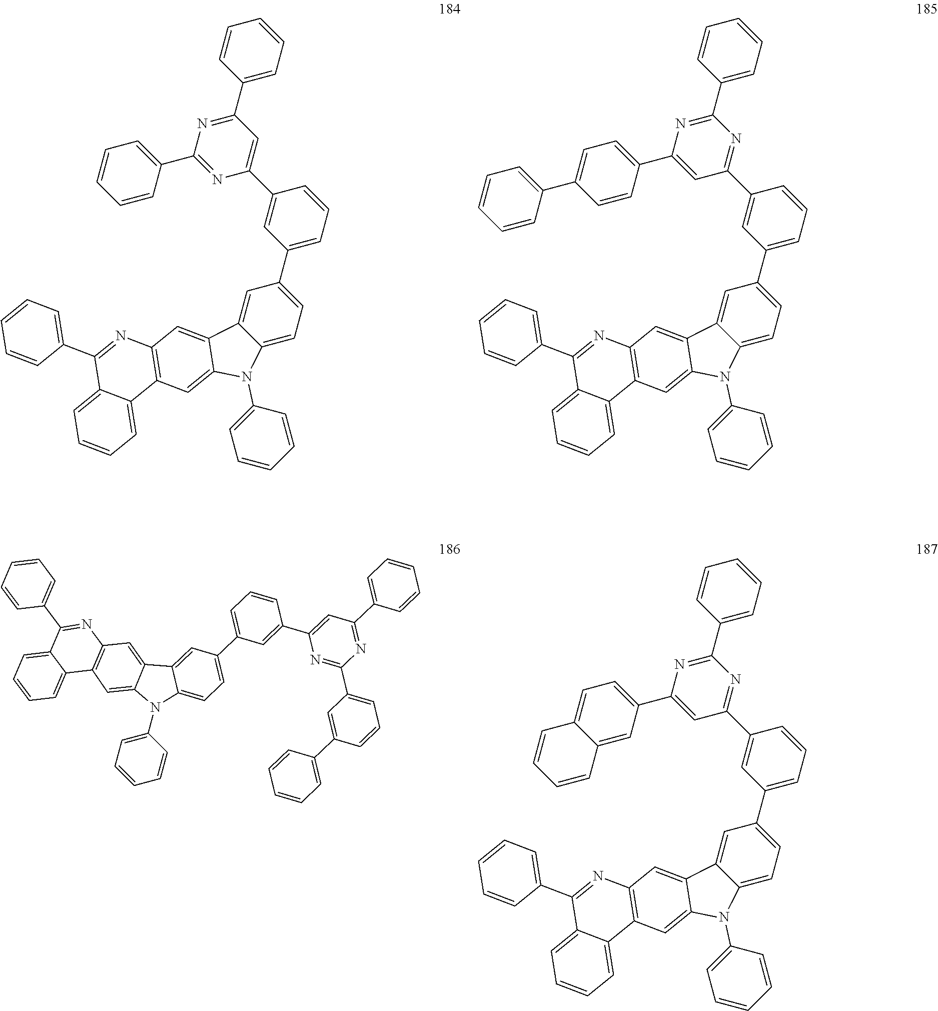 [SMILES: c1ccc(-c2cc(-c3cccc(-c4ccc5c(c4)c4cc6nc(-c7ccccc7)c7ccccc7c6cc4n5-c4ccccc4)c3)nc(-c3ccccc3)n2)cc1 ; c1ccc(-c2ccc(-c3cc(-c4cccc(-c5ccc6c(c5)c5cc7nc(-c8ccccc8)c8ccccc8c7cc5n6-c5ccccc5)c4)nc(-c4ccccc4)n3)cc2)cc1 ; c1ccc(-c2cccc(-c3nc(-c4ccccc4)cc(-c4cccc(-c5ccc6c(c5)c5cc7nc(-c8ccccc8)c8ccccc8c7cc5n6-c5ccccc5)c4)n3)c2)cc1 ; c1ccc(-c2nc(-c3cccc(-c4ccc5c(c4)c4cc6nc(-c7ccccc7)c7ccccc7c6cc4n5-c4ccccc4)c3)cc(-c3ccc4ccccc4c3)n2)cc1]

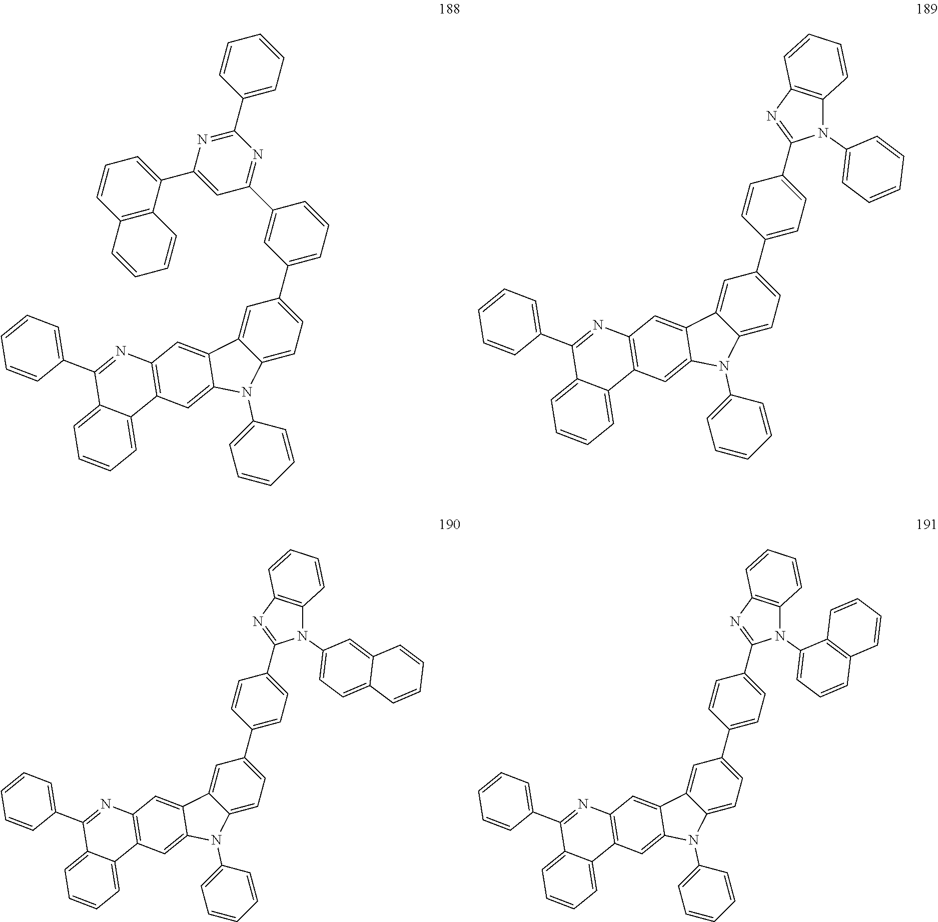 [SMILES: c1ccc(-c2nc(-c3cccc(-c4ccc5c(c4)c4cc6nc(-c7ccccc7)c7ccccc7c6cc4n5-c4ccccc4)c3)cc(-c3cccc4ccccc34)n2)cc1 ; c1ccc(-c2nc3cc4c5cc(-c6ccc(-c7nc8ccccc8n7-c7ccccc7)cc6)ccc5n(-c5ccccc5)c4cc3c3ccccc23)cc1 ; c1ccc(-c2nc3cc4c5cc(-c6ccc(-c7nc8ccccc8n7-c7ccc8ccccc8c7)cc6)ccc5n(-c5ccccc5)c4cc3c3ccccc23)cc1 ; c1ccc(-c2nc3cc4c5cc(-c6ccc(-c7nc8ccccc8n7-c7cccc8ccccc78)cc6)ccc5n(-c5ccccc5)c4cc3c3ccccc23)cc1]

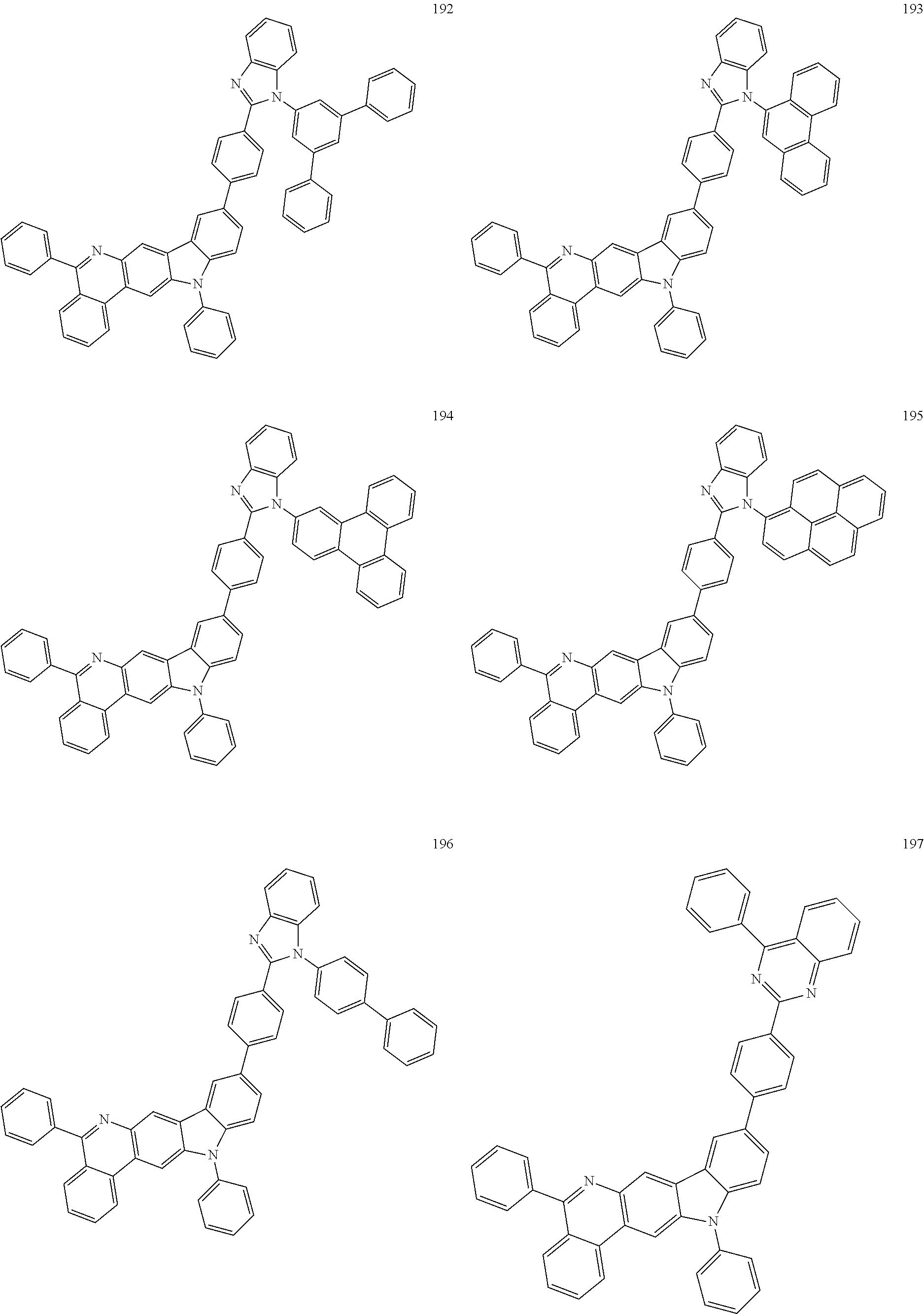

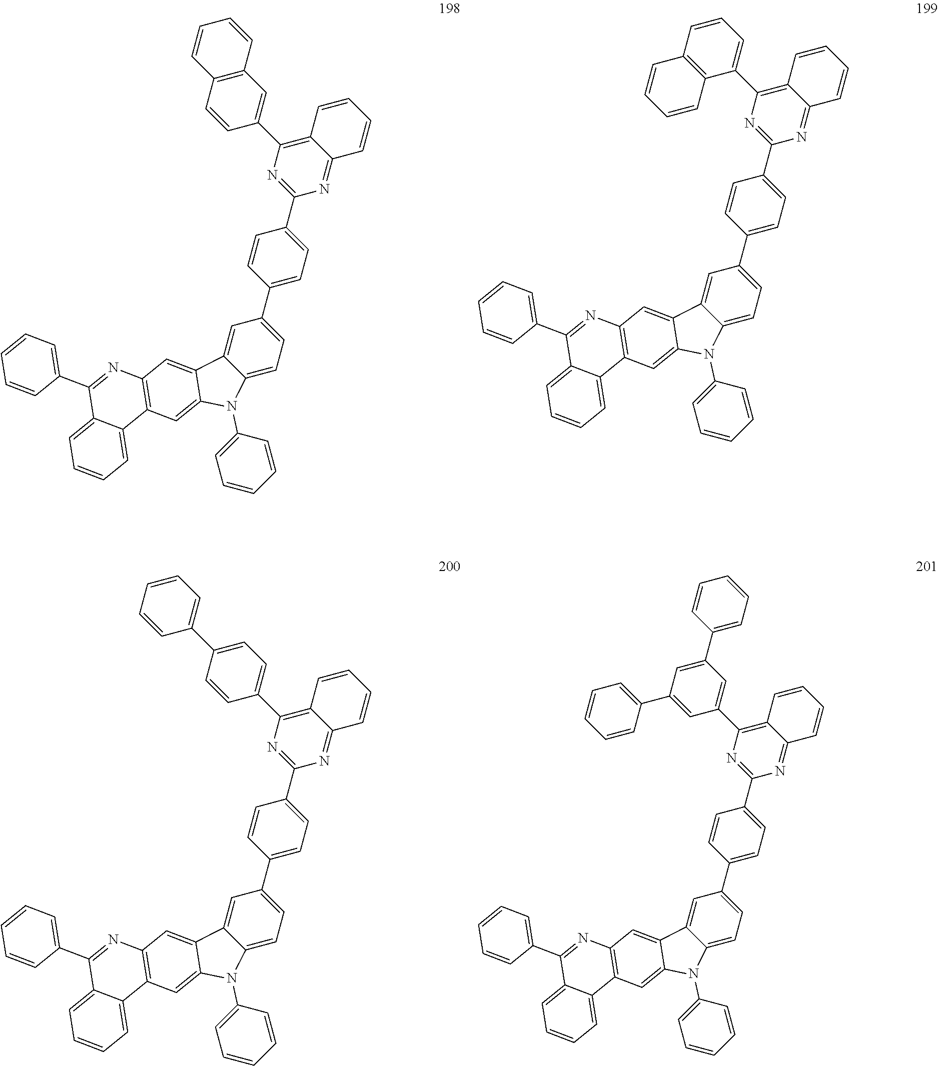 [SMILES: c1ccc(-c2nc3cc4c5cc(-c6ccc(-c7nc(-c8ccc9ccccc9c8)c8ccccc8n7)cc6)ccc5n(-c5ccccc5)c4cc3c3ccccc23)cc1 ; c1ccc(-c2nc3cc4c5cc(-c6ccc(-c7nc(-c8cccc9ccccc89)c8ccccc8n7)cc6)ccc5n(-c5ccccc5)c4cc3c3ccccc23)cc1 ; c1ccc(-c2ccc(-c3nc(-c4ccc(-c5ccc6c(c5)c5cc7nc(-c8ccccc8)c8ccccc8c7cc5n6-c5ccccc5)cc4)nc4ccccc34)cc2)cc1 ; c1ccc(-c2cc(-c3ccccc3)cc(-c3nc(-c4ccc(-c5ccc6c(c5)c5cc7nc(-c8ccccc8)c8ccccc8c7cc5n6-c5ccccc5)cc4)nc4ccccc34)c2)cc1]

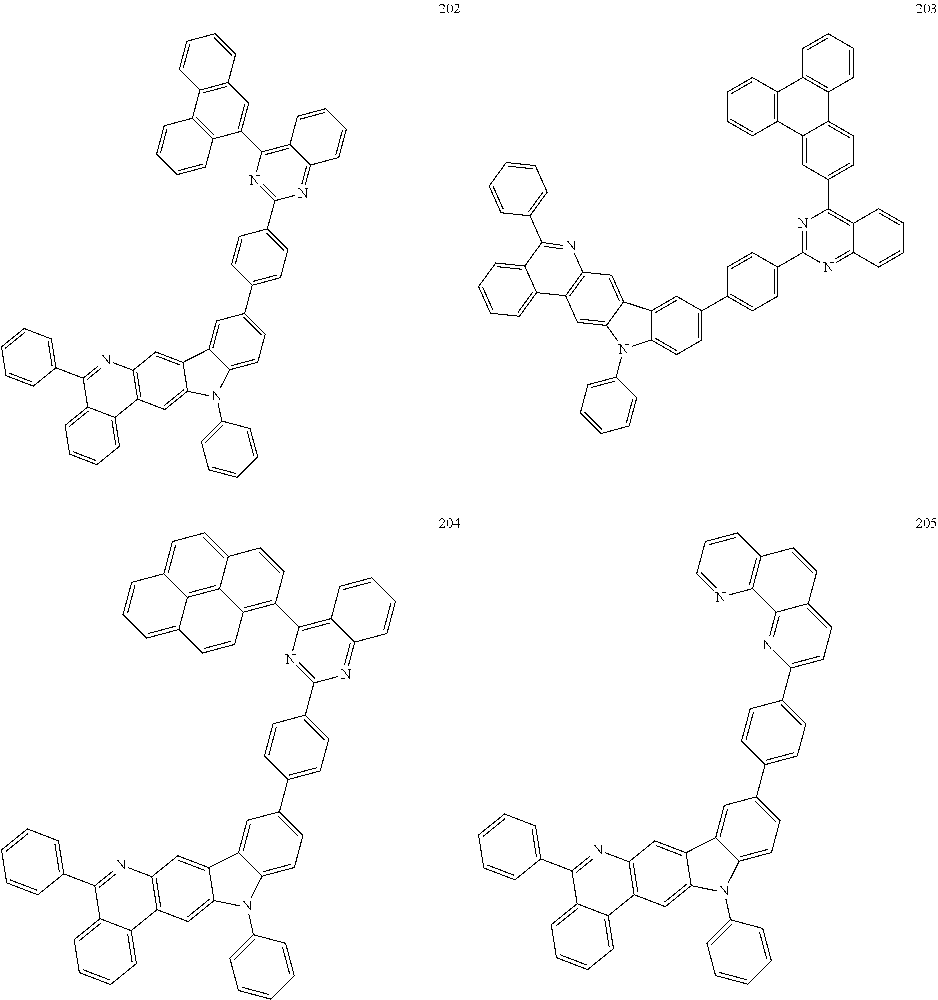 [SMILES: c1ccc(-c2nc3cc4c5cc(-c6ccc(-c7nc(-c8cc9ccccc9c9ccccc89)c8ccccc8n7)cc6)ccc5n(-c5ccccc5)c4cc3c3ccccc23)cc1 ; c1ccc(-c2nc3cc4c5cc(-c6ccc(-c7nc(-c8ccc9c%10ccccc%10c%10ccccc%10c9c8)c8ccccc8n7)cc6)ccc5n(-c5ccccc5)c4cc3c3ccccc23)cc1 ; c1ccc(-c2nc3cc4c5cc(-c6ccc(-c7nc(-c8ccc9ccc%10cccc%11ccc8c9c%10%11)c8ccccc8n7)cc6)ccc5n(-c5ccccc5)c4cc3c3ccccc23)cc1 ; c1ccc(-c2nc3cc4c5cc(-c6ccc(-c7ccc8ccc9cccnc9c8n7)cc6)ccc5n(-c5ccccc5)c4cc3c3ccccc23)cc1]

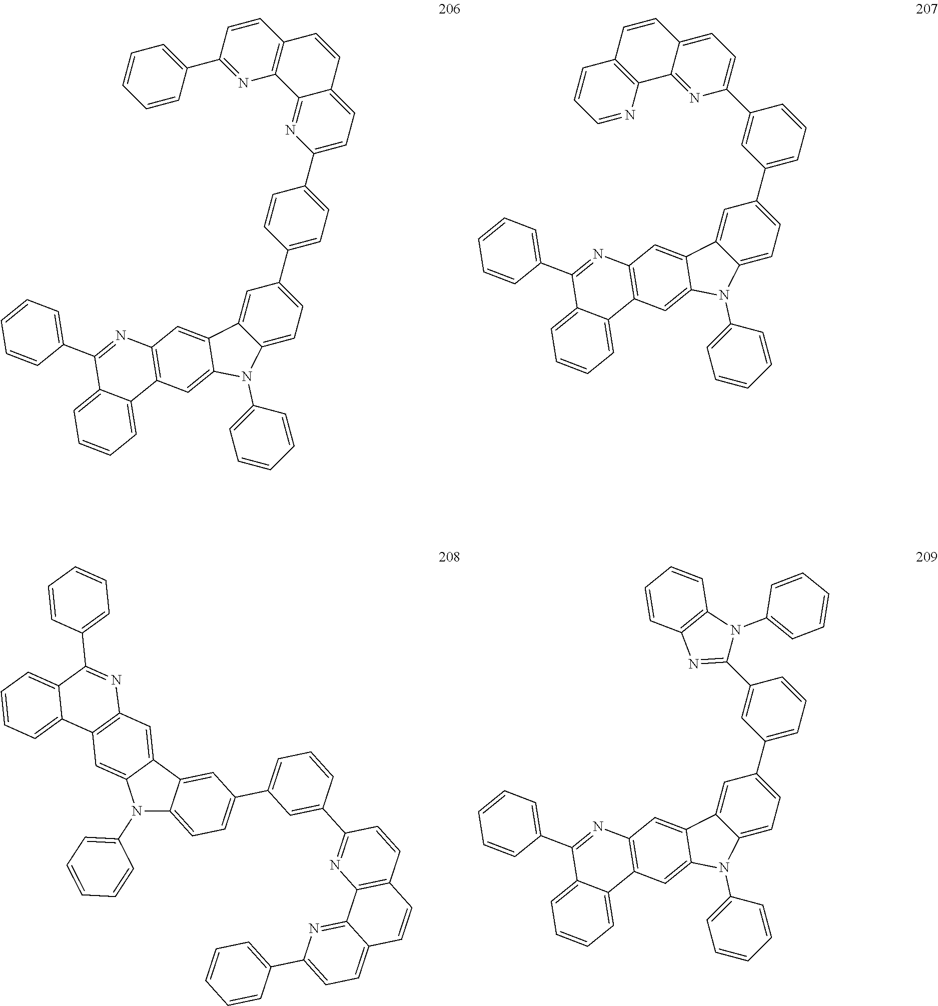 [SMILES: c1ccc(-c2ccc3ccc4ccc(-c5ccc(-c6ccc7c(c6)c6cc8nc(-c9ccccc9)c9ccccc9c8cc6n7-c6ccccc6)cc5)nc4c3n2)cc1 ; c1ccc(-c2nc3cc4c5cc(-c6cccc(-c7ccc8ccc9cccnc9c8n7)c6)ccc5n(-c5ccccc5)c4cc3c3ccccc23)cc1 ; c1ccc(-c2ccc3ccc4ccc(-c5cccc(-c6ccc7c(c6)c6cc8nc(-c9ccccc9)c9ccccc9c8cc6n7-c6ccccc6)c5)nc4c3n2)cc1 ; c1ccc(-c2nc3cc4c5cc(-c6cccc(-c7nc8ccccc8n7-c7ccccc7)c6)ccc5n(-c5ccccc5)c4cc3c3ccccc23)cc1]

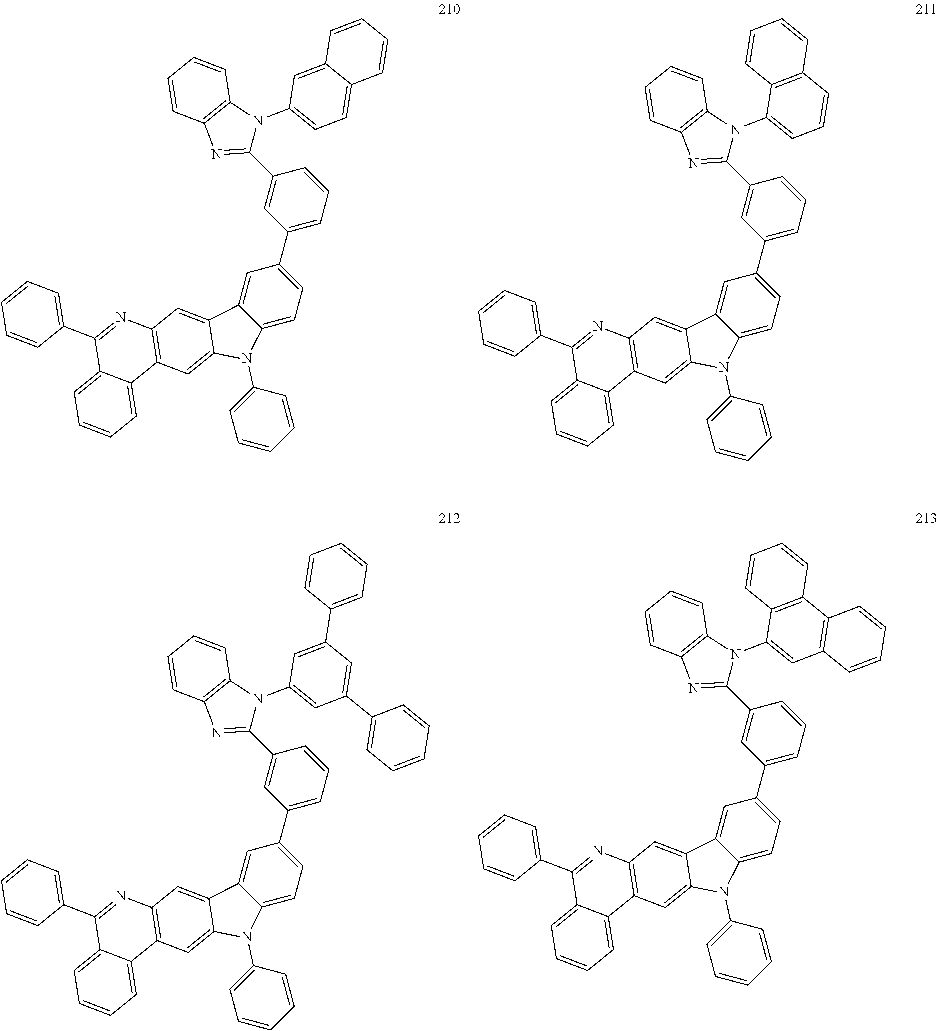 [SMILES: c1ccc(-c2nc3cc4c5cc(-c6cccc(-c7nc8ccccc8n7-c7ccc8ccccc8c7)c6)ccc5n(-c5ccccc5)c4cc3c3ccccc23)cc1 ; c1ccc(-c2nc3cc4c5cc(-c6cccc(-c7nc8ccccc8n7-c7cccc8ccccc78)c6)ccc5n(-c5ccccc5)c4cc3c3ccccc23)cc1 ; c1ccc(-c2cc(-c3ccccc3)cc(-n3c(-c4cccc(-c5ccc6c(c5)c5cc7nc(-c8ccccc8)c8ccccc8c7cc5n6-c5ccccc5)c4)nc4ccccc43)c2)cc1 ; c1ccc(-c2nc3cc4c5cc(-c6cccc(-c7nc8ccccc8n7-c7cc8ccccc8c8ccccc78)c6)ccc5n(-c5ccccc5)c4cc3c3ccccc23)cc1]

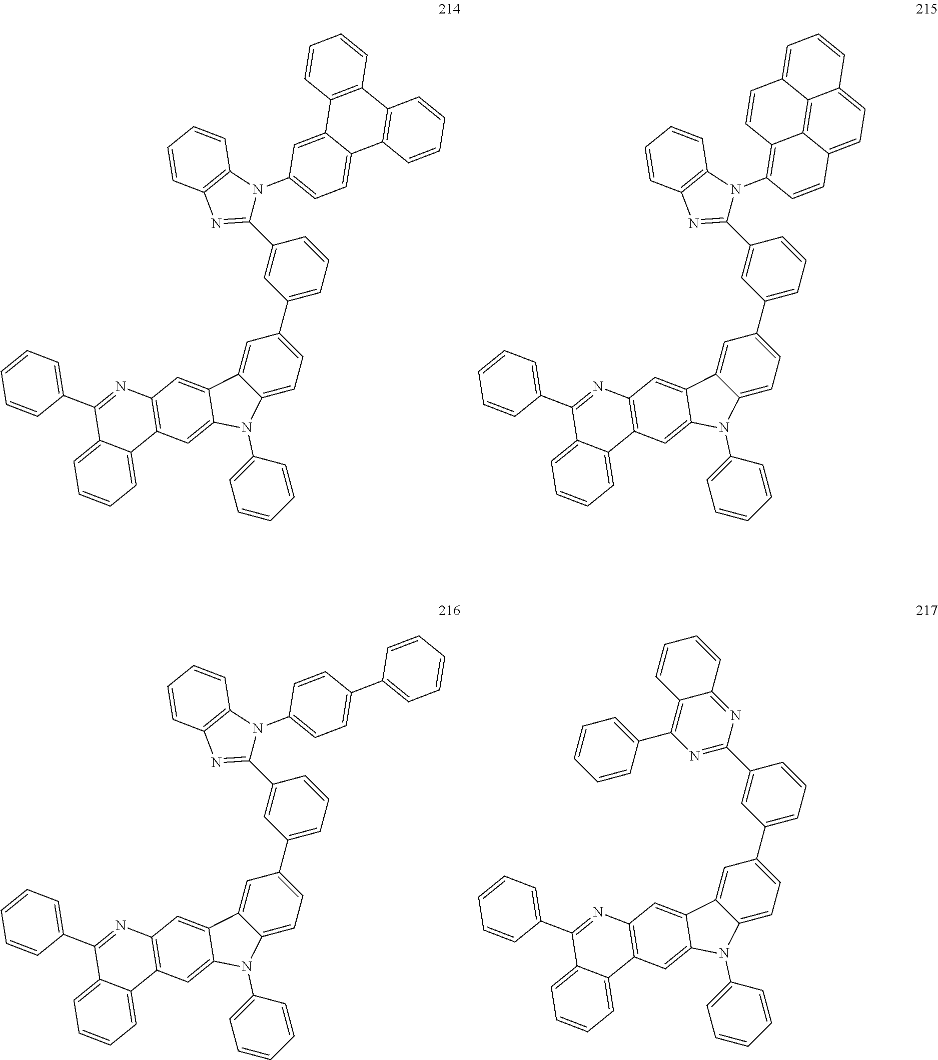 [SMILES: c1ccc(-c2nc3cc4c5cc(-c6cccc(-c7nc8ccccc8n7-c7ccc8c9ccccc9c9ccccc9c8c7)c6)ccc5n(-c5ccccc5)c4cc3c3ccccc23)cc1 ; c1ccc(-c2nc3cc4c5cc(-c6cccc(-c7nc8ccccc8n7-c7ccc8ccc9cccc%10ccc7c8c9%10)c6)ccc5n(-c5ccccc5)c4cc3c3ccccc23)cc1 ; c1ccc(-c2ccc(-n3c(-c4cccc(-c5ccc6c(c5)c5cc7nc(-c8ccccc8)c8ccccc8c7cc5n6-c5ccccc5)c4)nc4ccccc43)cc2)cc1 ; c1ccc(-c2nc(-c3cccc(-c4ccc5c(c4)c4cc6nc(-c7ccccc7)c7ccccc7c6cc4n5-c4ccccc4)c3)nc3ccccc23)cc1]

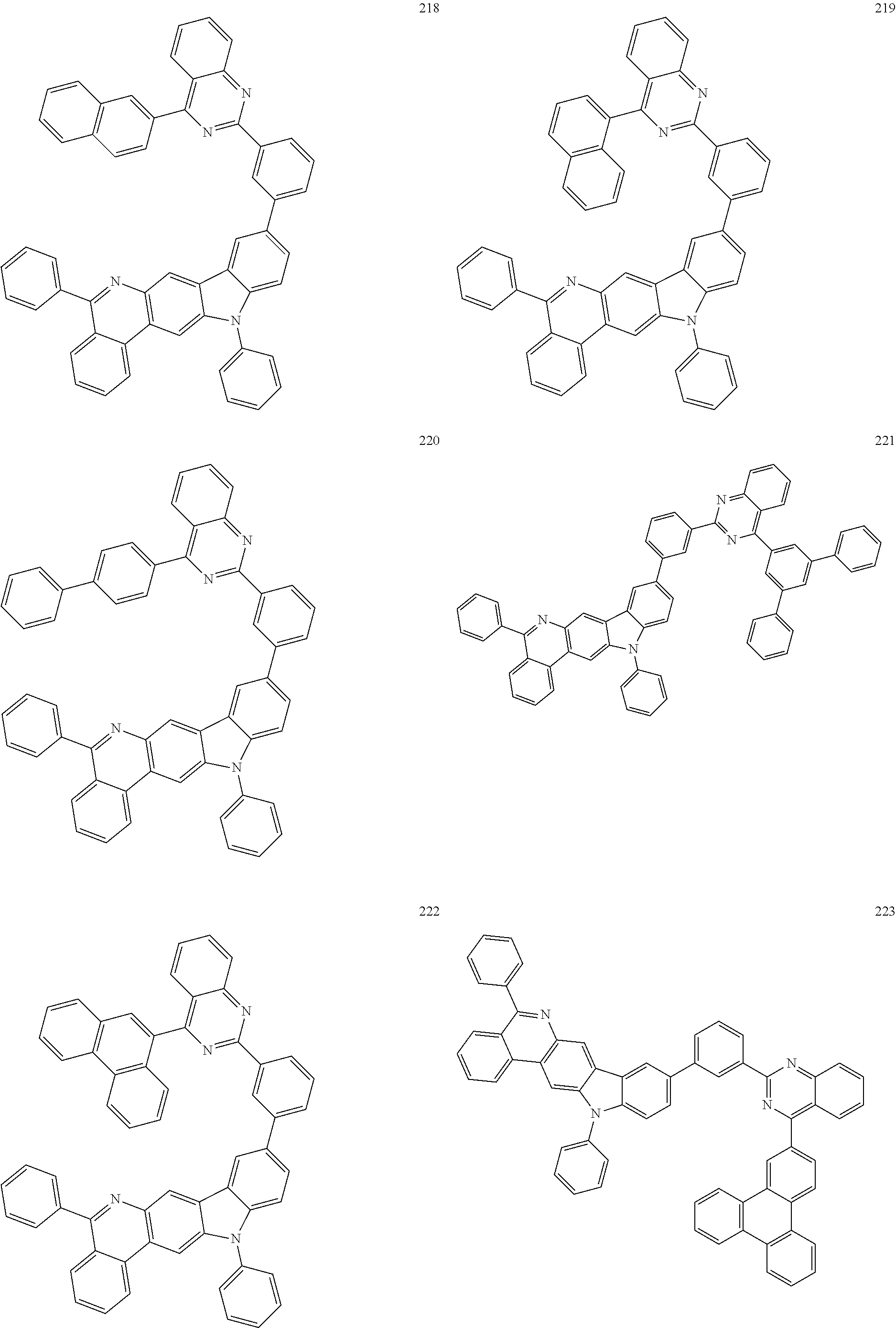

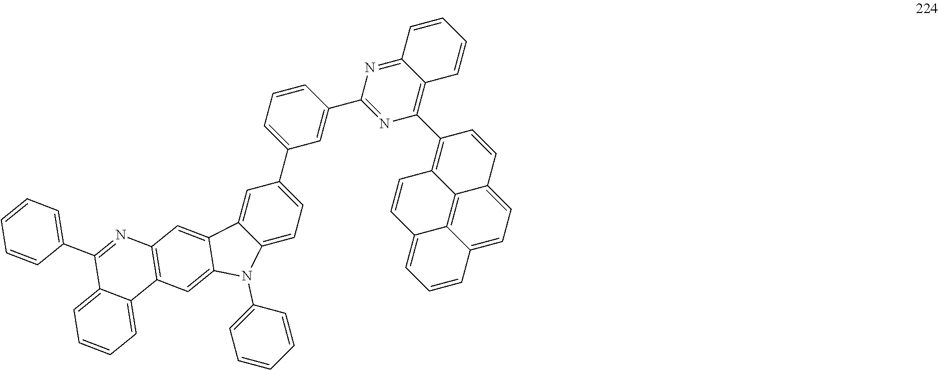 [SMILES: c1ccc(-c2nc3cc4c5cc(-c6cccc(-c7nc(-c8ccc9ccc%10cccc%11ccc8c9c%10%11)c8ccccc8n7)c6)ccc5n(-c5ccccc5)c4cc3c3ccccc23)cc1]

The organic compound may be used, for example, as a charge transport material and/or as a host material in the emitting layer, from among the materials used for an organic light emitting diode. Specifically, the organic compound may be used as a charge transport material, and preferably, as an electron transport material. In this case, excellent electron transport ability, stability, and efficiency may be achieved.

When the organic compound is used as a charge transport material, satisfactory charge transport characteristics may be obtained without a substantial increase in the driving voltage, and the light emission efficiency, stability, and lifetime of the organic light emitting diode may be ultimately improved through the provision of a stable and efficient charge transport material.

The organic light emitting diode of the present invention may comprise, for example: an anode; a cathode; and one or more layered organic thin film layer interposed between the anode and the cathode, wherein at least one layer of the organic thin film layer may comprise the charge transport material.

The organic thin film layer may comprise at least one selected from the group consisting of a hole injecting layer, a hole transport layer, an emitting layer, an electron transport layer, and an electron injecting layer.

In the organic light emitting diode, at least one layer of the organic thin film layer comprises the organic compound of the present invention, and the organic compound may be used for any one material from among the organic thin film layer, selected from the group consisting of a hole transport material, an electron transport material, the host material of an emitting layer, and a combination thereof.

Hereinafter, the organic light emitting diode is described in detail. However, it should be understood that the following exemplified description is an example only, and does not limit the organic light emitting diode of the present invention.

The organic light emitting diode may have, for example, a structure in which an anode (a hole injecting electrode); a hole injecting layer (HIL) and/or a hole transport layer (HTL); an emitting layer (EML); and a cathode (electron injecting electrode) are sequentially stacked, and preferably, may further comprise: an electron blocking layer (EBL) between the anode and the emitting layer; and an electron transport layer (ETL), an electron injecting layer (EIL) or a hole blocking layer (HBL) between the cathode and the emitting layer.

As an example of a method of manufacturing the organic light emitting diode, an anode is first formed by coating an anode material on the surface of a substrate using a conventional method. Here, the substrate to be used is preferably a glass substrate or a transparent plastic substrate with excellent transparency, surface smoothness, ease of handling, and waterproofness. Further, the anode material may be a material generally used in the art, and may be, for example, indium tin oxide (ITO), indium zinc oxide (IZO), tin oxide (SnO₂), zinc oxide (ZnO), or the like.

Next, the hole injecting material is subjected to vacuum thermal deposition or spin coating onto the surface of the anode using a conventional method, thereby forming the hole injecting layer.

When the hole injecting layer is formed by the vacuum thermal deposition method, the deposition conditions vary depending on the compound used as the hole injecting layer material, the structure and thermal properties of the hole injecting layer, and the like. For example, the deposition temperature may be selected in the range of 100 to 400° C., the vacuum pressure may be selected in the range of 10⁻⁸ to 10⁻⁶ torr, and the deposition rate may be selected in the range of 0.01 to 2 Å/sec, but the deposition conditions are not limited thereto.

Further, when the hole injecting layer is formed by the spin coating method, the coating conditions vary depending on the compound used as the hole injecting layer material, and the structure and thermal properties of the hole injecting layer. For example, the coating speed may be 2000 to 5000 rpm, and the heat treatment temperature for removing the solvent after coating may be in the range of 80 to 200° C., but the coating conditions are not limited thereto.

Examples of the hole injecting material include copper phthalocyanine (CuPc), 4,4′,4″-tris(3-methylphenylamino)triphenylamine (m-MTDATA), 4,4′,4″-tris(3-methylphenylamino)phenoxybenzene (m-MTDAPB), and starburst amines such as 4,4′,4″-tri(N-carbazolyl)triphenylamine (TCTA), 4,4′,4″-tris(N-(2-naphthyl)-N-phenylamino)-triphenylamine (2-TNATA), IDE406 (available from Idemitsu Kosan Co., Ltd., Tokyo, Japan), and the like. The thickness of the hole injecting layer may be, for example, 100 to 10,000 Å or 100 to 2,000 Å, and preferably 100 to 1,000 Å. Within this range, satisfactory hole injection characteristics may be obtained without a substantial increase in the driving voltage.

Next, the hole transport material is subjected to vacuum thermal deposition or spin coating onto a surface of the hole injecting layer using a conventional method, thereby forming the hole transport layer. Here, when the organic compound of the present invention is used as the hole transport material, the light emission efficiency, stability, and lifetime of the organic light emitting diode may be improved. In addition, as the hole transport material, bis(N-(1-naphthyl-n-phenyl))benzidine (α-NPD), N′-di(naphthalene-1-yl)-N,N′-biphenyl-benzidine (NPB), N,N′-biphenyl-N,N′-bis(3-methylphenyl)-1,1′-biphenyl-4,4′-diamine (TPD), or the like, may be used. The thickness of the hole transport layer may be, for example, 100 to 10,000 Å or 100 to 1,500 Å, and preferably 100 to 3,000 Å. Within this range, satisfactory hole transport characteristics may be obtained without a substantial increase in the driving voltage.

The thickness of each layer in the present description may be measured through a measurement method generally recognized in the art, and as a specific example, the thickness of each layer may be measured using a probe profilometer.

Next, an emitting layer (EML) material is subjected to vacuum thermal deposition or spin coating onto a surface of the hole transport layer using a conventional method, thereby forming the emitting layer. Here, when the organic compound of the present invention is used as an emitting host material from among the emitting layer materials to be used, the light emission efficiency, stability, and lifetime of the organic light emitting diode may be improved. In addition, as the emitting layer material, tris(8-hydroxyquinolinolato)aluminum (Alq₃), 8-hydroxyquinoline beryllium salt (Balq), a 4,4′-bis(2,2-biphenylethenyl)-1,1′-biphenyl)-based material (DPVBi), a spiro material, spiro-4,4′-bis(2,2-biphenylethenyl)-1,1′-biphenyl (spiro-DPVBi), 2-(2-benzooxazolyl)-phenollithium salt (LiPBO), bis(biphenylvinyl)benzene, an aluminum-quinoline metal complex, metal complexes of imidazole, thiazole, and oxazole, and the like, may be used.

Among the emitting layer materials, in the case of a dopant capable of being used together with an emitting host, IDE102 and IDE105 (available from Idemitsu Kosan Co., Ltd., Tokyo, Japan) may be used as fluorescent dopants, while tris(2-phenylpyridine)iridium (III) (Ir(ppy)₃), iridium (III) bis[(4,6-difluorophenyl)pyridinato-N,C2′]picolinate (FIrpic), platinum (II) octaethylporphyrin (PtOEP), TBE002 (manufactured by Corbion), and the like, may be used as phosphorescent dopants.

When the emitting layer comprises a host and a dopant, the content of the dopant may be, for example, 1 to 15 parts by weight, 1 to 10 parts by weight, and preferably 1 to 5 parts by weight, based on 100 parts by weight of the host. Within this range, it is possible to improve the light emission efficiency, stability, and lifetime of the organic light emitting diode.

The thickness of the hole transport layer may be, for example, 100 to 10,000 Å or 200 to 1,500 Å, and preferably 100 to 3,000 Å. Within this range, satisfactory emission characteristics can be obtained without a substantial increase in the driving voltage.

As an example, the electron transport material may be deposited by vacuum thermal deposition or spin coated onto the surface of the emitting layer, thereby forming the electron transport layer. Here, when the organic compound of the present invention is used as the electron transport material, the light emission efficiency, stability, and lifetime of the organic light emitting diode may be improved. Further, the electron transport layer may further comprise, for example, a metal-containing material such as tris(8-hydroxyquinolinolato)aluminum (Alq3), 2-(4-biphenylyl)-5(4-tert-butylphenyl)-1,3,4-oxadiazole (PBD), 2,4,7-trinitro fluorenone (TNF), BMD, BND, or the like, in addition to the organic compound of the present invention. The thickness of the electron transport layer may be, for example, 100 to 1,000 Å or 150 to 500 Å, and preferably 100 to 500 Å. Within this range, satisfactory electron transport characteristics may be obtained without a substantial increase in the driving voltage. The metal-containing material may be used together with, for example, a Li complex. Non-limiting examples of the Li complex may include lithium quinolate (LiQ), and the like.

By selectively forming a hole blocking layer between the emitting layer and the electron transport layer, and together using a phosphorescent dopant in the emitting layer, it is possible to prevent a phenomenon in which triplet excitons or holes are diffused into the electron transport layer. The hole blocking layer may be formed by vacuum thermal deposition or spin coating with a hole blocking layer material using a conventional method. The hole blocking layer material that may be used is not particularly limited, but it should have an ionization potential higher than that of the light emitting compound while having electron transport ability. For example, the hole blocking layer material may be (8-hydroxyquinolinolato)lithium (Liq), bis(8-hydroxy-2-methylquinolinolato)-aluminum biphenoxide (BAlq), bathocuproine (BCP), or the like.

As an example, the electron injecting material is subjected to vacuum thermal deposition or spin coating onto a surface of the electron transport layer using a conventional method, thereby forming the electron injecting layer. The electron injecting material to be used in this case is not particularly limited as long as it is a material generally used in the art, and may be, for example, LiF, Liq, Li₂O, BaO, CsF, or the like. The thickness of the electron injecting layer may be, for example, 1 to 100 Å, 3 to 90 Å, and preferably 5 to 50 Å. Within this range, satisfactory electron injecting characteristics can be obtained without a substantial increase in the driving voltage.

As an example, a cathode material is deposited by vacuum thermal deposition onto the surface of the electron injecting layer using a conventional method, thereby forming the cathode. The cathode material to be used in this case may be lithium (Li), aluminum (Al), aluminum-lithium (Al—Li), calcium (Ca), magnesium (Mg), magnesium-indium (Mg—In), magnesium-silver (Mg—Ag), or the like. In addition, in the case of a top emission organic electroluminescent device, for example, indium tin oxide (ITO) or indium zinc oxide (IZO) may be used to form a transparent cathode capable of transmitting light.

The organic light emitting diode according to the present invention may be manufactured in the order described above, that is, in the order of anode/hole injecting layer/hole transport layer/emitting layer/hole blocking layer/electron transport layer/electron injecting layer/and cathode, and may also be manufactured in the reverse order, that is, in the order of cathode/electron injecting layer/electron transport layer/hole blocking layer/emitting layer/hole transport layer/hole injecting layer/anode.

A display device of the present invention is characterized by comprising the organic light emitting diode as described above. The display device may be, for example, a portable device (a portable computer, an electronic organizer, a watch, a remote control, or a camcorder), a road sign, the display portion of an electronic device, or a wall-mounted TV, and in this case, the desired effect is well exhibited.

Hereinafter, a method of synthesizing the organic compounds of the present invention is described below with reference to representative examples. However, the method of synthesizing the compounds of the present invention is not limited to the following exemplified methods, and the compounds of the present invention may be prepared by the methods exemplified below as well as by other methods known in the art.

### Preparation Example: Synthesis of the Organic Compound

### Compound of the Present Invention

### Preparation Example

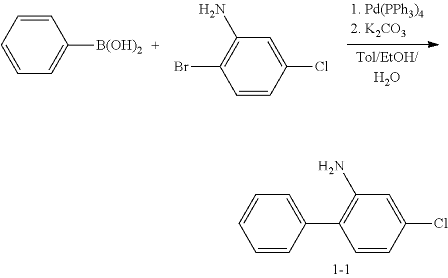 [SMILES: OB(O)c1ccccc1 ; Nc1cc(Cl)ccc1Br ; Nc1cc(Cl)ccc1-c1ccccc1]

<Preparation of Core 1-1>

A 10 L round-bottom flask was charged with 200 g (1.64 mol, 1.05 eq.) of Phenylboronic acid, 322 g (1.56 mol, 1 eq.) of 2-bromo-5-chloroaniline, 90 g (78.10 mmol, 0.05 eq.) of Pd(PPh₃)₄, and 646 g (4.69 mol, 3 eq.) of K₂CO₃, after which toluene/EtOH/H₂O was added thereto, and the mixture was stirred under reflux.

After completion of the reaction, the mixture was extracted with MC/H₂O and the MC layer was dried over MgSO₄. Purification was performed using a silica-gel column to obtain 254 g of Core 1-1 at a yield of 80%.

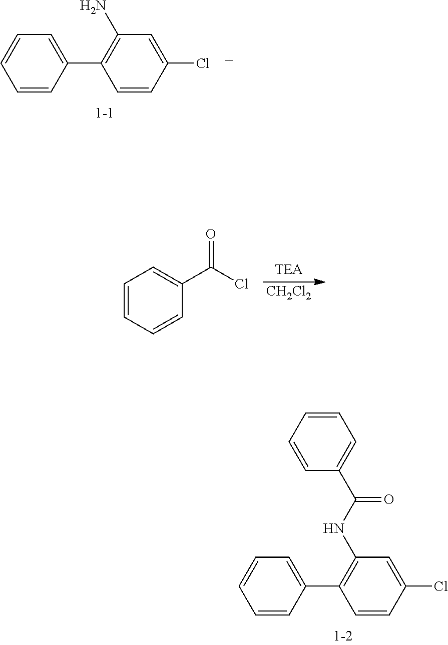

<Preparation of Core 1-2>

A 10 L round-bottom flask was charged with 200 g (983.42 mmol, 1 eq.) of Core 1-1 and 119 g (1180.11 mmol, 1.2 eq.) of TEA, and the mixture was dissolved in CH₂Cl₂, followed by cooling to 0° C. To the cooled mixture, 152 g (1081.77 mmol, 1.1 eq.) of benzoylchloride was added dropwise, and the mixture was heated to room temperature and stirred.

After completion of the reaction, the mixture was extracted with MC/H₂O and the MC layer was dried over MgSO₄. Purification was performed using a silica-gel column to obtain 271 g of Core 1-2 at a yield of 90%.

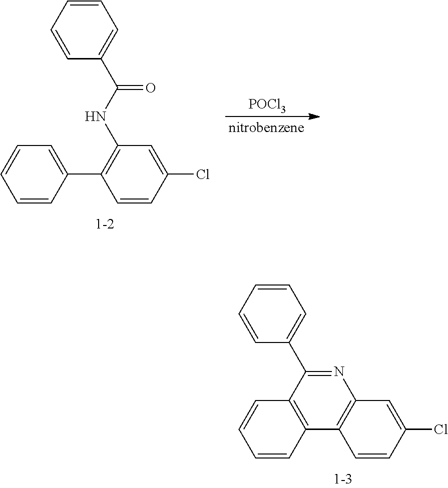 [SMILES: O=C(Nc1cc(Cl)ccc1-c1ccccc1)c1ccccc1 ; "POCl3" ; Clc1ccc2c(c1)nc(-c1ccccc1)c1ccccc12]

<Preparation of Core 1-3>

A 10 L round-bottom flask was charged with 200 g (651.46 mmol, 1 eq.) of Core 1-2 and 199 g (1302.93 mmol, 2 eq.) of POCl₃, then nitrobenzene was added thereto, and the mixture was stirred at 150° C.

After completion of the reaction, the mixture was extracted with MC/H₂O and the MC layer was dried over MgSO₄. Purification was performed using a silica-gel column to obtain 131 g of Core 1-3 at a yield of 70%.

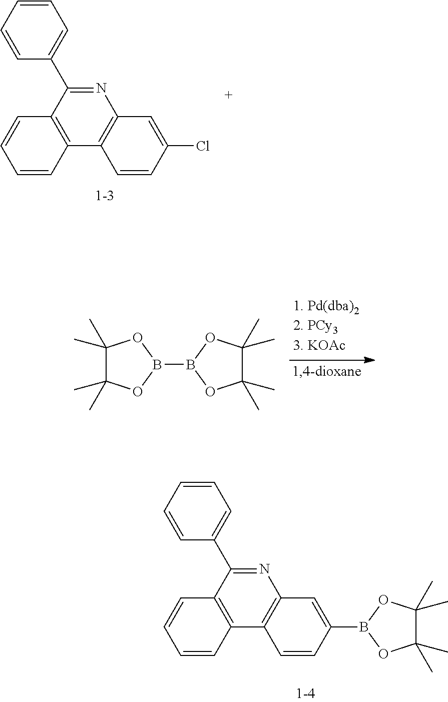

<Preparation of Core 1-4>

A 5 L round-bottom flask was charged with 131 g (452.09 mmol, 1 eq.) of Core 1-3, 172 g (678.14 mmol, 1.5 eq.) of bis(pinacolato)diboron, 13 g (22.60 mmol, 0.05 eq.) of Pd(dba)₂, 12 g (45.20 mmol, 0.1 eq.) of PCy₃, and 177.5 g (1808.36 mmol, 4 eq.) of KOAc, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 138 g of Core 1-4 at a yield of 80%.

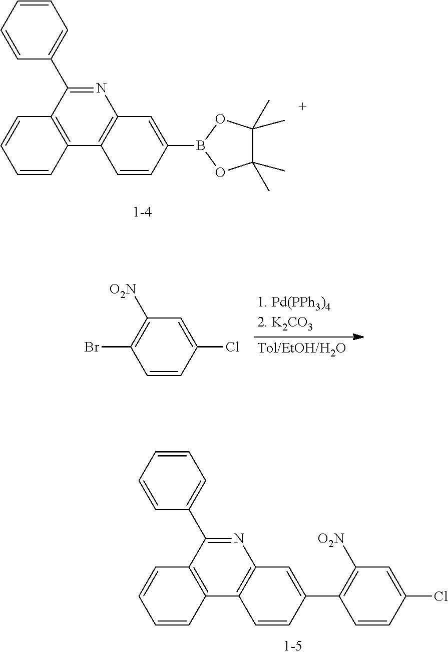 [SMILES: CC1(C)OB(c2ccc3c(c2)nc(-c2ccccc2)c2ccccc23)OC1(C)C ; O=[N+]([O-])c1cc(Cl)ccc1Br ; O=[N+]([O-])c1cc(Cl)ccc1-c1ccc2c(c1)nc(-c1ccccc1)c1ccccc12]

<Preparation of Core 1-5>

A 1 L round-bottom flask was charged with 30 g (78.68 mmol, 1 eq.) of Core 1-4, 18.6 g (78.68 mmol, 1 eq.) of 1-bromo-4-chloro-2-nitrobenzene, 4.6 g (3.93 mmol, 0.05 eq.) of Pd(PPh₃)₄, and 32.6 g (236.05 mmol, 3 eq.) of K₂CO₃, then toluene/EtOH/H₂O was added thereto, and the mixture was stirred under reflux.

After completion of the reaction, the mixture was extracted with MC/H₂O and the MC layer was dried over MgSO₄. Purification was performed using a silica-gel column to obtain 29 g of Core 1-5 at a yield of 90%.

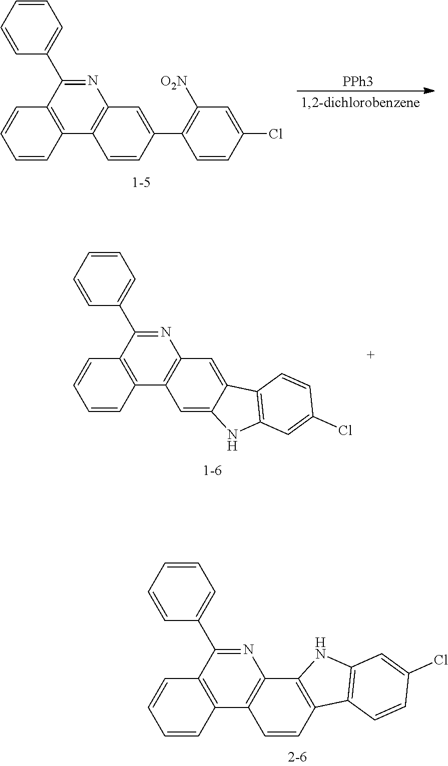 [SMILES: O=[N+]([O-])c1cc(Cl)ccc1-c1ccc2c(c1)nc(-c1ccccc1)c1ccccc12 ; Clc1ccc2c(c1)[nH]c1cc3c(cc12)nc(-c1ccccc1)c1ccccc13 ; Clc1ccc2c(c1)[nH]c1c2ccc2c3ccccc3c(-c3ccccc3)nc21 ; "PPh3"]

<Preparation of Core 1-6 and 2-6>

A 500 mL round-bottom flask was charged with 29 g (70.58 mmol, 1 eq.) of Core 1-5, and 37 g (141.17 mmol, 2 eq.) of PPh₃, then 1,2-dichlorobenzene was added thereto, and the mixture was stirred under reflux.

After completion of the reaction, the mixture was extracted with MC/H₂O and the MC layer was dried over MgSO₄. Purification was performed using a silica-gel column to obtain 15 g of Core 1-6 at a yield of 56%, and 9 g of Core 2-6 at a yield of 34%.

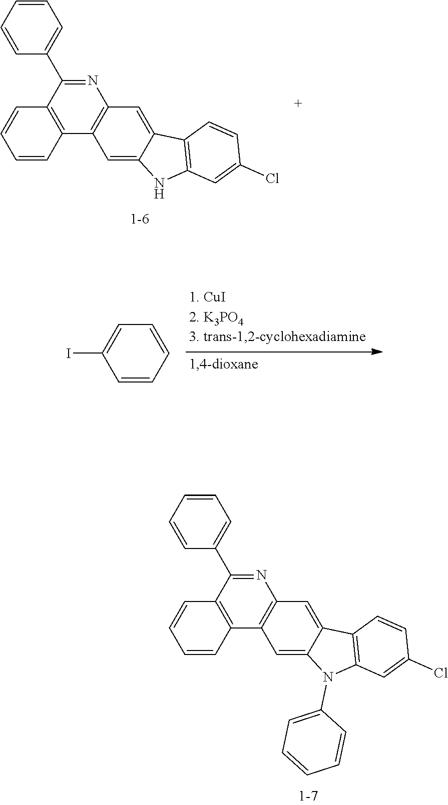 [SMILES: Clc1ccc2c(c1)[nH]c1cc3c(cc12)nc(-c1ccccc1)c1ccccc13 ; Ic1ccccc1 ; Clc1ccc2c3cc4nc(-c5ccccc5)c5ccccc5c4cc3n(-c3ccccc3)c2c1]

<Preparation of Core 1-7>

A 1 L round-bottom flask was charged with 9 g (23.76 mmol, 1 eq.) of Core 1-6, 5.3 g (26.13 mmol, 1.1 eq.) of iodobenzene, 2.3 g (11.88 mmol, 0.5 eq.) of CuI, 15.1 g (71.27 mmol, 3 eq.) of K₃PO₄, and 4.1 g (35.63 mmol, 1.5 eq.) of trans-1,2-cyclohexadiamine, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 9 g of Core 1-7 at a yield of 83%.

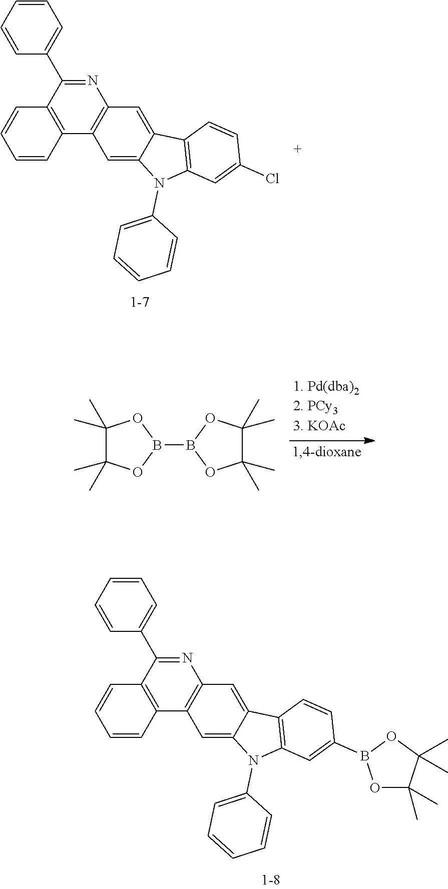

<Preparation of Core 1-8>

A 500 mL round-bottom flask was charged with 9 g (19.78 mmol, 1 eq.) of Core 1-7, 7.5 g (29.67 mmol, 1.5 eq.) of bis(pinacolato)diboron, 0.6 g (0.99 mmol, 0.05 eq.) of Pd(dba)₂, 0.6 g (1.98 mmol, 0.1 eq.) of PCy₃, and 7.8 g (79.13 mmol, 4 eq.) of KOAc, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 8.3 g of Core 1-8 at a yield of 77%.

<General Procedure A>

A 500 ml round-bottom flask was charged with Core 1-8 (1.1 eq.), Ar1-X (1 eq.) as described in Table 1 below, Pd(PPh₃)₄ (0.05 eq.) and K₂CO₃ (3 eq.), then toluene/EtOH/was added thereto, and the mixture was stirred under reflux.

After completion of the reaction, the mixture was extracted with MC/H₂O and the MC layer was dried over MgSO₄. Purification was performed using a silica-gel column to obtain a material.

TABLE 1

| Com- | | Yield | |
|---|---|---|---|
| pound | Ar1-X | (%) | Structure |
| 113 | 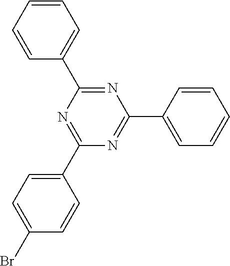 | 81 | 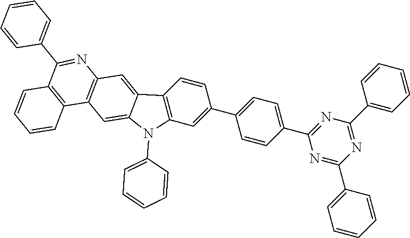 [SMILES: c1ccc(-c2nc(-c3ccccc3)nc(-c3ccc(-c4ccc5c6cc7nc(-c8ccccc8)c8ccccc8c7cc6n(-c6ccccc6)c5c4)cc3)n2)cc1] |
| 118 | 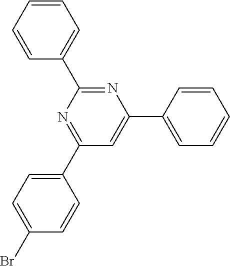 | 78 | 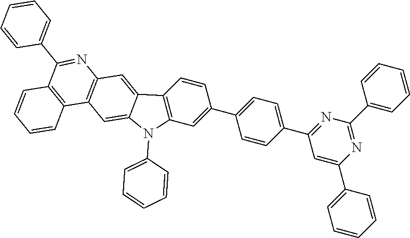 [SMILES: c1ccc(-c2cc(-c3ccc(-c4ccc5c6cc7nc(-c8ccccc8)c8ccccc8c7cc6n(-c6ccccc6)c5c4)cc3)nc(-c3ccccc3)n2)cc1] |
| 119 | 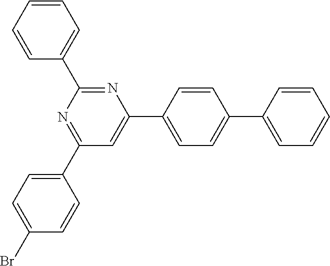 | 84 | 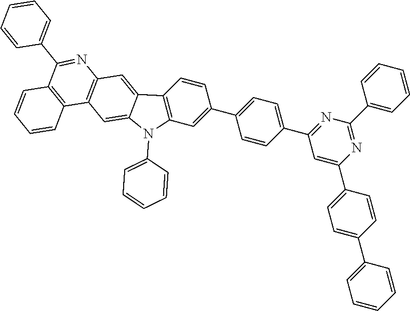 [SMILES: c1ccc(-c2ccc(-c3cc(-c4ccc(-c5ccc6c7cc8nc(-c9ccccc9)c9ccccc9c8cc7n(-c7ccccc7)c6c5)cc4)nc(-c4ccccc4)n3)cc2)cc1] |
| 123 | 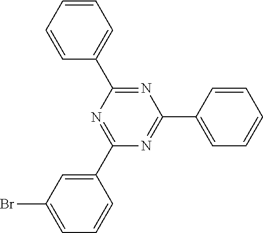 | 81 | 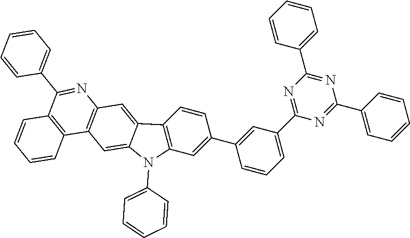 [SMILES: c1ccc(-c2nc(-c3ccccc3)nc(-c3cccc(-c4ccc5c6cc7nc(-c8ccccc8)c8ccccc8c7cc6n(-c6ccccc6)c5c4)c3)n2)cc1] |
| 128 | 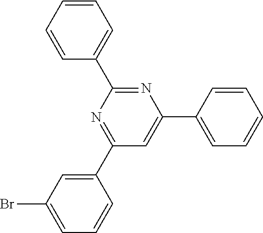 | 70 | 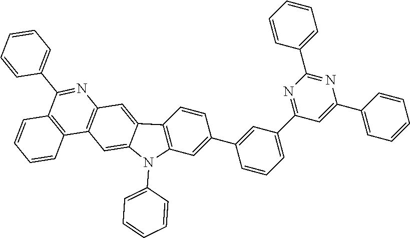 [SMILES: c1ccc(-c2cc(-c3cccc(-c4ccc5c6cc7nc(-c8ccccc8)c8ccccc8c7cc6n(-c6ccccc6)c5c4)c3)nc(-c3ccccc3)n2)cc1] |
| 129 | 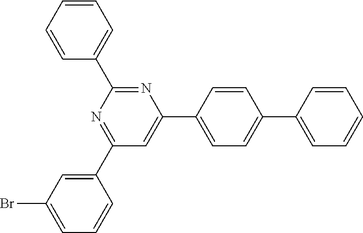 | 89 | 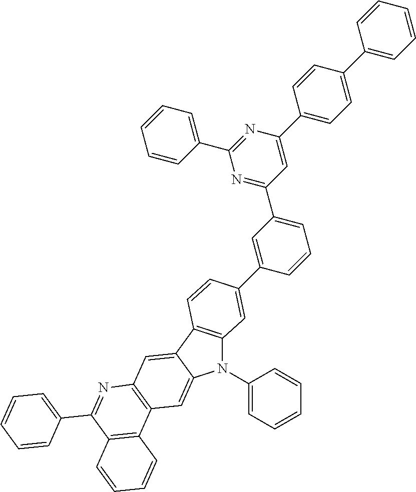 [SMILES: c1ccc(-c2ccc(-c3cc(-c4cccc(-c5ccc6c7cc8nc(-c9ccccc9)c9ccccc9c8cc7n(-c7ccccc7)c6c5)c4)nc(-c4ccccc4)n3)cc2)cc1] |

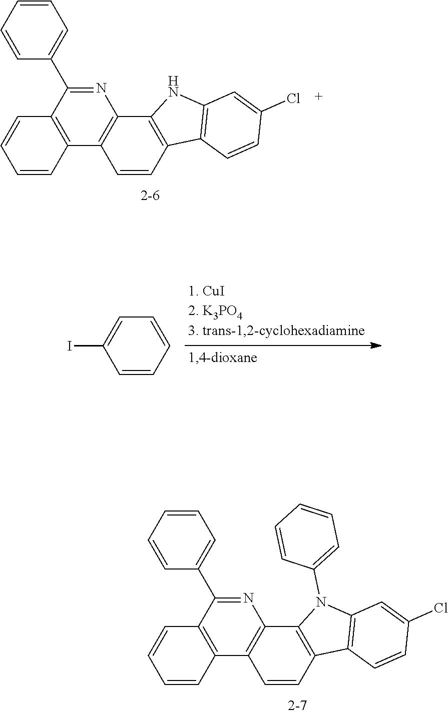 [SMILES: Clc1ccc2c(c1)[nH]c1c2ccc2c3ccccc3c(-c3ccccc3)nc21 ; Ic1ccccc1 ; Clc1ccc2c3ccc4c5ccccc5c(-c5ccccc5)nc4c3n(-c3ccccc3)c2c1]

<Preparation of Core 2-7>

A 1 L round-bottom flask was charged with 9 g (23.76 mmol, 1 eq.) of Core 2-6, 5.3 g (26.13 mmol, 1.1 eq.) of iodobenzene, 2.3 g (11.88 mmol, 0.5 eq.) of CuI, 15.1 g (71.27 mmol, 3 eq.) of K₃PO₄, and 4.1 g (35.63 mmol, 1.5 eq.) of trans-1,2-cyclohexadiamine, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 8.1 g of Core 2-7 at a yield of 75%.

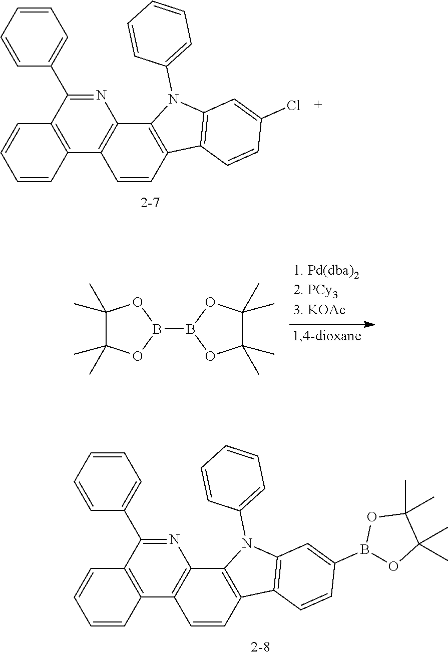

<Preparation of Core 2-8>

A 500 mL round-bottom flask was charged with 9 g (19.78 mmol, 1 eq.) of Core 2-7, 7.5 g (29.67 mmol, 1.5 eq.) of bis(pinacolato)diboron, 0.6 g (0.99 mmol, 0.05 eq.) of Pd(dba)₂, 0.6 g (1.98 mmol, 0.1 eq.) of PCy₃, and 7.8 g (79.13 mmol, 4 eq.) of KOAc, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 9 g of Core 2-8 at a yield of 83%.

<General Procedure B>

A 500 ml round-bottom flask was charged with Core 2-8 (1.1 eq.), Ar1-X (1 eq.) as described in Table 2 below, Pd(PPh₃)₄ (0.05 eq.) and K₂CO₃ (3 eq.), then toluene/EtOH/was added thereto, and the mixture was stirred under reflux.

After completion of the reaction, the mixture was extracted with MC/H₂O and the MC layer was dried over MgSO₄. Purification was performed using a silica-gel column to obtain a material.

TABLE 2

| Com- | | Yield | |
|---|---|---|---|
| pound | Ar1-X | (%) | Structure |
| 1 | 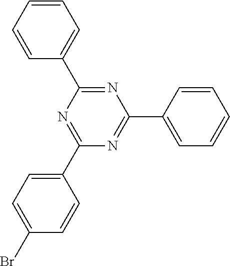 | 87 | 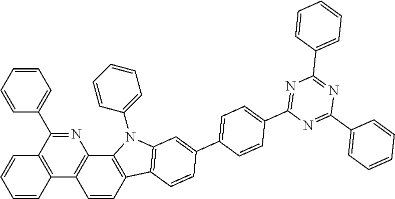 [SMILES: c1ccc(-c2nc(-c3ccccc3)nc(-c3ccc(-c4ccc5c6ccc7c8ccccc8c(-c8ccccc8)nc7c6n(-c6ccccc6)c5c4)cc3)n2)cc1] |
| 6 | 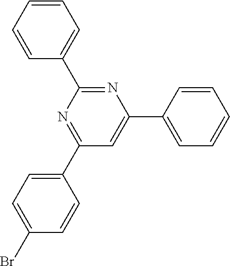 | 78 | 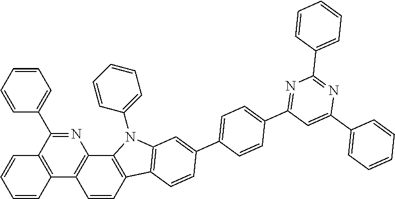 [SMILES: c1ccc(-c2cc(-c3ccc(-c4ccc5c6ccc7c8ccccc8c(-c8ccccc8)nc7c6n(-c6ccccc6)c5c4)cc3)nc(-c3ccccc3)n2)cc1] |
| 7 | 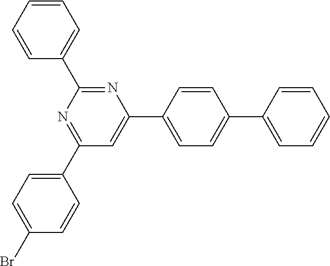 | 90 | 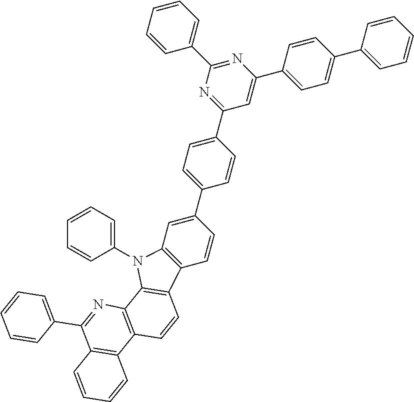 [SMILES: c1ccc(-c2ccc(-c3cc(-c4ccc(-c5ccc6c7ccc8c9ccccc9c(-c9ccccc9)nc8c7n(-c7ccccc7)c6c5)cc4)nc(-c4ccccc4)n3)cc2)cc1] |
| 11 | 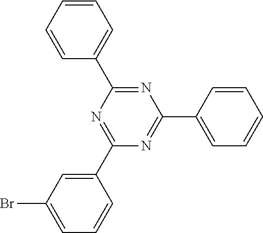 | 83 | 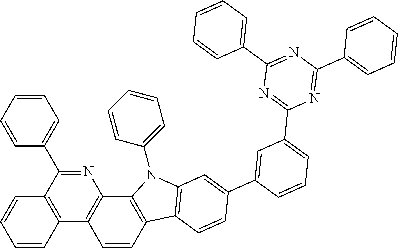 [SMILES: c1ccc(-c2nc(-c3ccccc3)nc(-c3cccc(-c4ccc5c6ccc7c8ccccc8c(-c8ccccc8)nc7c6n(-c6ccccc6)c5c4)c3)n2)cc1] |
| 16 | 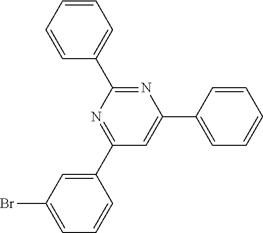 | 70 | 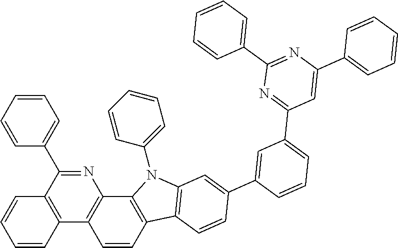 [SMILES: c1ccc(-c2cc(-c3cccc(-c4ccc5c6ccc7c8ccccc8c(-c8ccccc8)nc7c6n(-c6ccccc6)c5c4)c3)nc(-c3ccccc3)n2)cc1] |
| 17 | 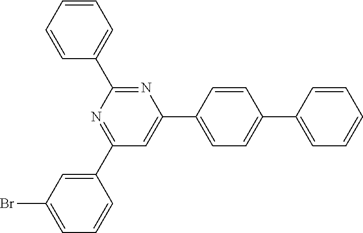 | 85 | 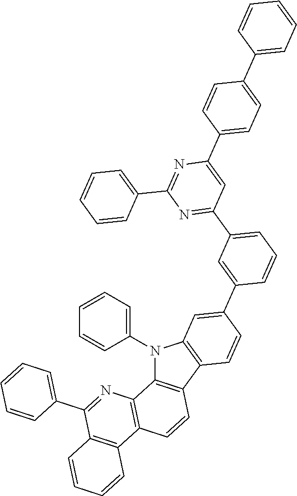 [SMILES: c1ccc(-c2ccc(-c3cc(-c4cccc(-c5ccc6c7ccc8c9ccccc9c(-c9ccccc9)nc8c7n(-c7ccccc7)c6c5)c4)nc(-c4ccccc4)n3)cc2)cc1] |

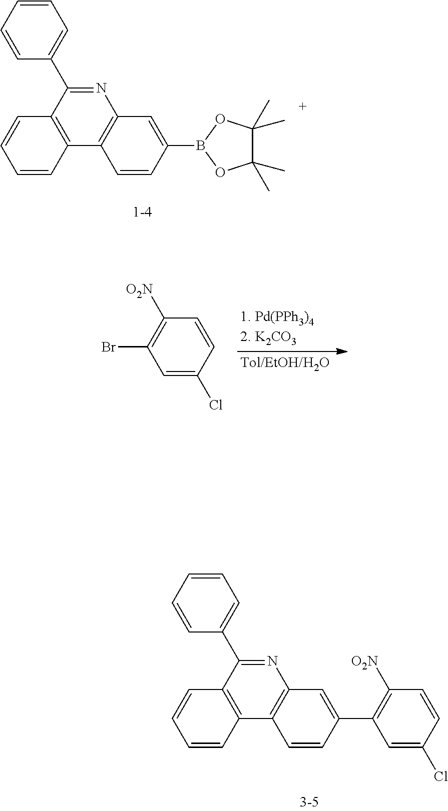 [SMILES: CC1(C)OB(c2ccc3c(c2)nc(-c2ccccc2)c2ccccc23)OC1(C)C ; O=[N+]([O-])c1ccc(Cl)cc1Br ; O=[N+]([O-])c1ccc(Cl)cc1-c1ccc2c(c1)nc(-c1ccccc1)c1ccccc12]

<Preparation of Core 3-5>

A 1 L round-bottom flask was charged with 30 g (78.68 mmol, 1 eq.) of Core 1-4, 18.6 g (78.68 mmol, 1 eq.) of 1-bromo-4-chloro-2-nitrobenzene, 4.6 g (3.93 mmol, 0.05 eq.) of Pd(PPh₃)₄, and 32.6 g (236.05 mmol, 3 eq.) of K₂CO₃, then toluene/EtOH/H₂O was added thereto, and the mixture was stirred under reflux.

After completion of the reaction, the mixture was extracted with MC/H₂O and the MC layer was dried over MgSO₄. Purification was performed using a silica-gel column to obtain 25 g of Core 3-5 at a yield of 77%.

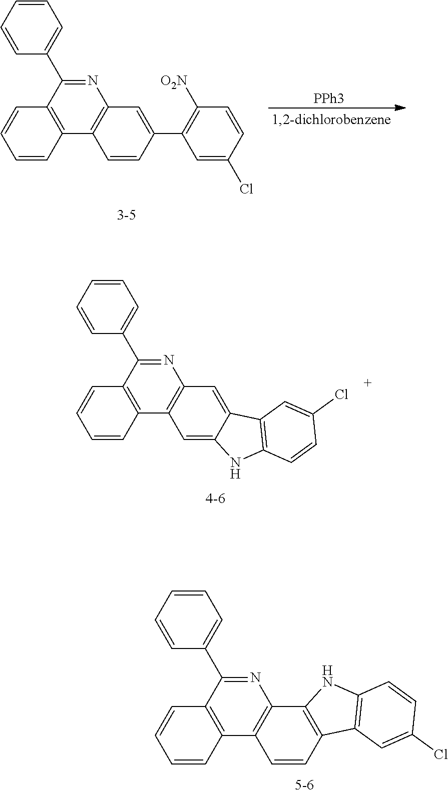 [SMILES: O=[N+]([O-])c1ccc(Cl)cc1-c1ccc2c(c1)nc(-c1ccccc1)c1ccccc12 ; Clc1ccc2[nH]c3cc4c(cc3c2c1)nc(-c1ccccc1)c1ccccc14 ; Clc1ccc2[nH]c3c(ccc4c5ccccc5c(-c5ccccc5)nc43)c2c1 ; "PPh3"]

<Preparation of Core 4-6 and 5-6>

A 500 mL round-bottom flask was charged with 25 g (60.85 mmol, 1 eq.) of Core 3-5, and 31.9 g (121.70 mmol, 2 eq.) of PPh₃, then 1,2-dichlorobenzene was added thereto, and the mixture was stirred under reflux.

After completion of the reaction, the mixture was extracted with MC/H₂O and the MC layer was dried over MgSO₄. Purification was performed using a silica-gel column to obtain 12 g of Core 4-6 at a yield of 52%, and 9 g of Core 5-6 at a yield of 39%.

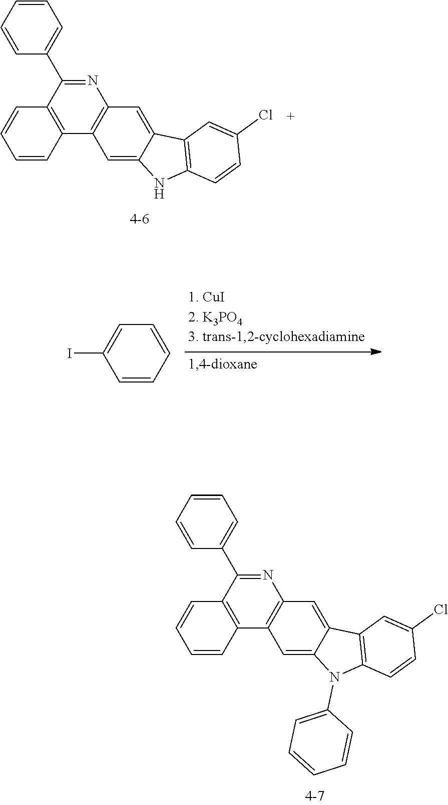 [SMILES: Clc1ccc2[nH]c3cc4c(cc3c2c1)nc(-c1ccccc1)c1ccccc14 ; Ic1ccccc1 ; Clc1ccc2c(c1)c1cc3nc(-c4ccccc4)c4ccccc4c3cc1n2-c1ccccc1]

<Preparation of Core 4-7>

A 1 L round-bottom flask was charged with 9 g (23.76 mmol, 1 eq.) of Core 4-6, 5.3 g (26.13 mmol, 1.1 eq.) of iodobenzene, 2.3 g (11.88 mmol, 0.5 eq.) of CuI, 15.1 g (71.27 mmol, 3 eq.) of K₃PO₄, and 4.1 g (35.63 mmol, 1.5 eq.) of trans-1,2-cyclohexadiamine, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 8.4 g of Core 4-7 at a yield of 78%.

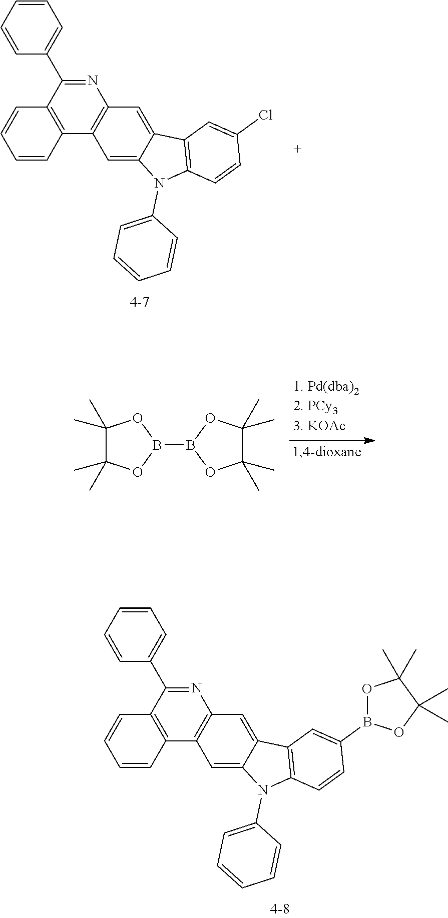

<Preparation of Core 4-8>

A 500 mL round-bottom flask was charged with 8.4 g (18.46 mmol, 1 eq.) of Core 4-7, 7 g (27.70 mmol, 1.5 eq.) of bis(pinacolato)diboron, 0.5 g (0.92 mmol, 0.05 eq.) of Pd(dba)₂, 0.5 g (1.85 mmol, 0.1 eq.) of PCy₃, and 7.3 g (73.85 mmol, 4 eq.) of KOAc, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 9 g of Core 4-8 at a yield of 89%.

<General Procedure C>

A 500 ml round-bottom flask was charged with Core 4-8 (1.1 eq.), Ar1-X (1 eq.) as described in Table 3 below, Pd(PPh₃)₄ (0.05 eq.) and K₂CO₃ (3 eq.), then toluene/EtOH/H₂O was added thereto, and the mixture was stirred under reflux.

After completion of the reaction, the mixture was extracted with MC/H₂O and the MC layer was dried over MgSO₄. Purification was performed using a silica-gel column to obtain a material.

TABLE 3

| Com- | | Yield | |
|---|---|---|---|
| pound | Ar1-X | (%) | Structure |
| 169 | 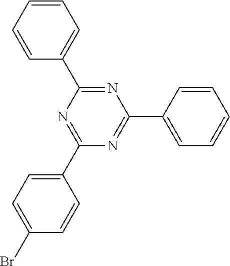 | 90 | 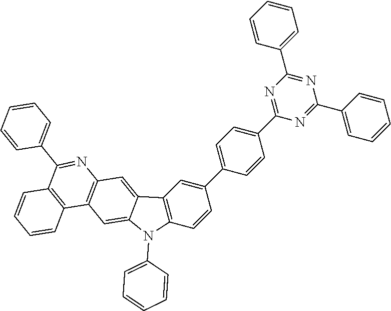 [SMILES: c1ccc(-c2nc(-c3ccccc3)nc(-c3ccc(-c4ccc5c(c4)c4cc6nc(-c7ccccc7)c7ccccc7c6cc4n5-c4ccccc4)cc3)n2)cc1] |
| 174 | 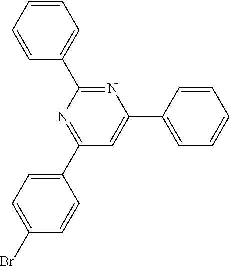 | 86 | 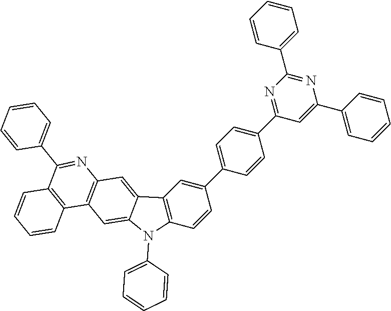 [SMILES: c1ccc(-c2cc(-c3ccc(-c4ccc5c(c4)c4cc6nc(-c7ccccc7)c7ccccc7c6cc4n5-c4ccccc4)cc3)nc(-c3ccccc3)n2)cc1] |
| 175 | 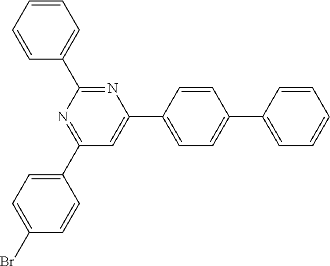 | 79 | 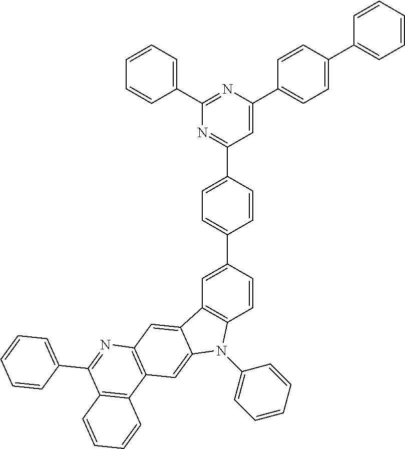 [SMILES: c1ccc(-c2ccc(-c3cc(-c4ccc(-c5ccc6c(c5)c5cc7nc(-c8ccccc8)c8ccccc8c7cc5n6-c5ccccc5)cc4)nc(-c4ccccc4)n3)cc2)cc1] |
| 179 | 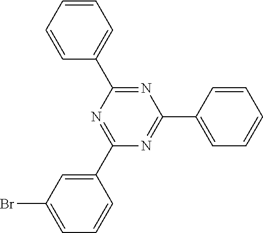 | 81 | 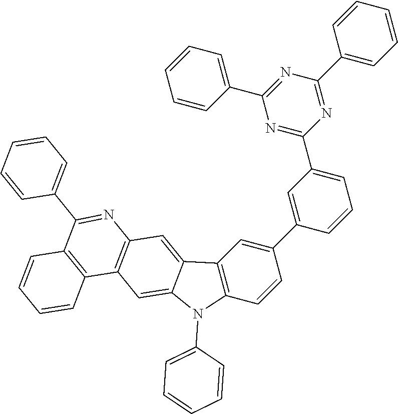 [SMILES: c1ccc(-c2nc(-c3ccccc3)nc(-c3cccc(-c4ccc5c(c4)c4cc6nc(-c7ccccc7)c7ccccc7c6cc4n5-c4ccccc4)c3)n2)cc1] |
| 184 | 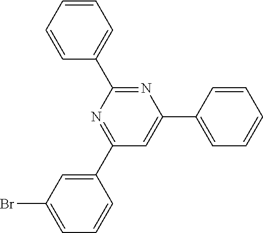 | 79 | 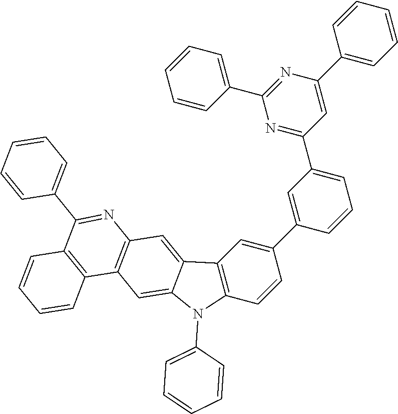 [SMILES: c1ccc(-c2cc(-c3cccc(-c4ccc5c(c4)c4cc6nc(-c7ccccc7)c7ccccc7c6cc4n5-c4ccccc4)c3)nc(-c3ccccc3)n2)cc1] |
| 185 | 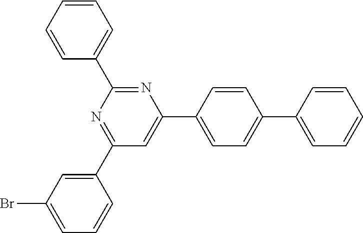 | 85 | 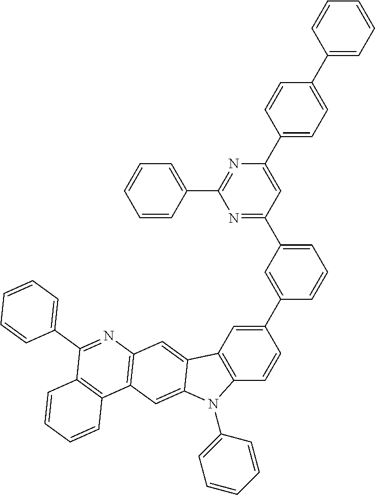 [SMILES: c1ccc(-c2ccc(-c3cc(-c4cccc(-c5ccc6c(c5)c5cc7nc(-c8ccccc8)c8ccccc8c7cc5n6-c5ccccc5)c4)nc(-c4ccccc4)n3)cc2)cc1] |

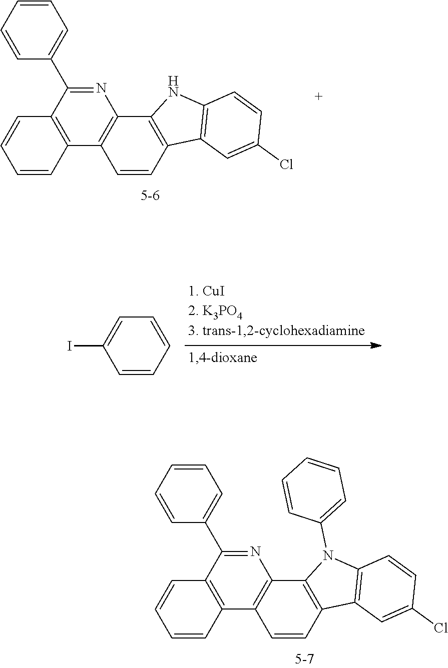 [SMILES: Clc1ccc2[nH]c3c(ccc4c5ccccc5c(-c5ccccc5)nc43)c2c1 ; Ic1ccccc1 ; Clc1ccc2c(c1)c1ccc3c4ccccc4c(-c4ccccc4)nc3c1n2-c1ccccc1]

<Preparation of Core 5-7>

A 1 L round-bottom flask was charged with 9 g (23.76 mmol, 1 eq.) of Core 5-6, 5.3 g (26.13 mmol, 1.1 eq.) of iodobenzene, 2.3 g (11.88 mmol, 0.5 eq.) of CuI, 15.1 g (71.27 mmol, 3 eq.) of K₃PO₄, and 4.1 g (35.63 mmol, 1.5 eq.) of trans-1,2-cyclohexadiamine, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 8 g of Core 5-7 at a yield of 74%.

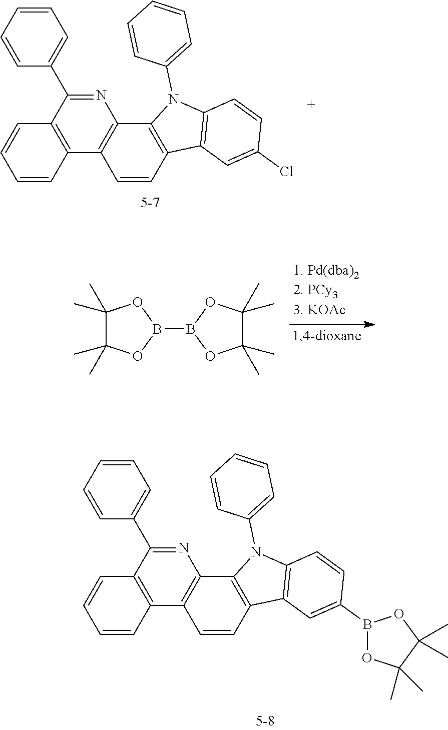

<Preparation of Core 5-8>

A 500 mL round-bottom flask was charged with 8 g (17.58 mmol, 1 eq.) of Core 5-7, 6.7 g (26.38 mmol, 1.5 eq.) of bis(pinacolato)diboron, 0.5 g (0.88 mmol, 0.05 eq.) of Pd(dba)₂, 0.5 g (1.76 mmol, 0.1 eq.) of PCy₃, and 6.9 g (70.34 mmol, 4 eq.) of KOAc, then 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 8.3 g of Core 5-8 at a yield of 87%.

<General Procedure D>

A 500 ml round-bottom flask was charged with Core 5-8 (1.1 eq.), Ar1-X (1 eq.) as described in Table 4 below, Pd(PPh₃)₄ (0.05 eq.) and K₂CO₃ (3 eq.), then toluene/EtOH/H₂O was added thereto, and the mixture was stirred under reflux.

After completion of the reaction, the mixture was extracted with MC/H₂O and the MC layer was dried over MgSO₄. Purification was performed using a silica-gel column to obtain a material.

TABLE 4

| Com- | | Yield | |
|---|---|---|---|
| pound | Ar1-X | (%) | Structure |
| 57 | 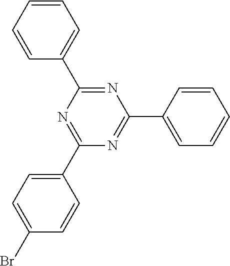 | 93 | 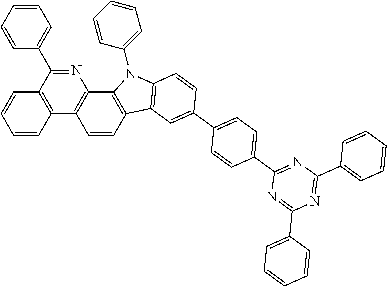 [SMILES: c1ccc(-c2nc(-c3ccccc3)nc(-c3ccc(-c4ccc5c(c4)c4ccc6c7ccccc7c(-c7ccccc7)nc6c4n5-c4ccccc4)cc3)n2)cc1] |
| 62 | 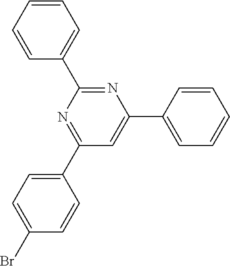 | 81 | 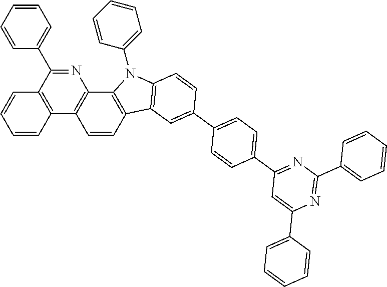 [SMILES: c1ccc(-c2cc(-c3ccc(-c4ccc5c(c4)c4ccc6c7ccccc7c(-c7ccccc7)nc6c4n5-c4ccccc4)cc3)nc(-c3ccccc3)n2)cc1] |
| 63 | 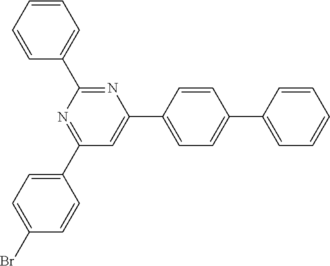 | 88 | 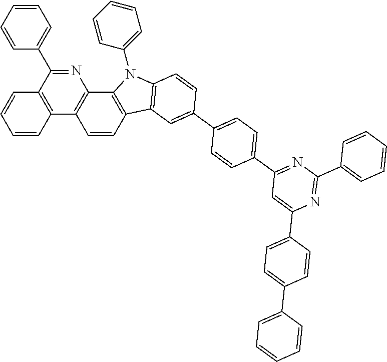 [SMILES: c1ccc(-c2ccc(-c3cc(-c4ccc(-c5ccc6c(c5)c5ccc7c8ccccc8c(-c8ccccc8)nc7c5n6-c5ccccc5)cc4)nc(-c4ccccc4)n3)cc2)cc1] |
| 67 | 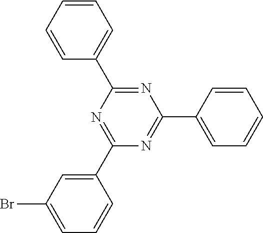 | 85 | 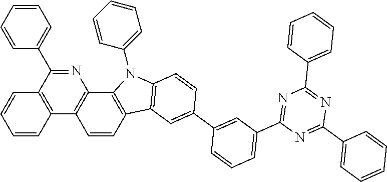 [SMILES: c1ccc(-c2nc(-c3ccccc3)nc(-c3cccc(-c4ccc5c(c4)c4ccc6c7ccccc7c(-c7ccccc7)nc6c4n5-c4ccccc4)c3)n2)cc1] |
| 72 | 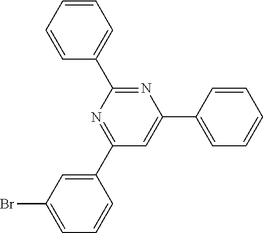 | 79 | 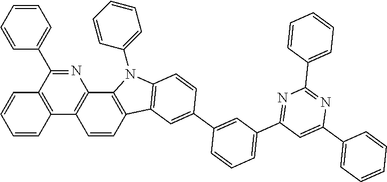 [SMILES: c1ccc(-c2cc(-c3cccc(-c4ccc5c(c4)c4ccc6c7ccccc7c(-c7ccccc7)nc6c4n5-c4ccccc4)c3)nc(-c3ccccc3)n2)cc1] |
| 73 | 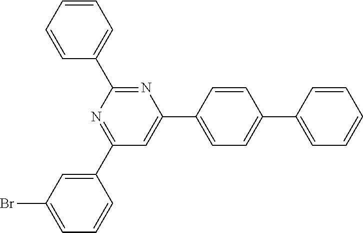 | 80 | 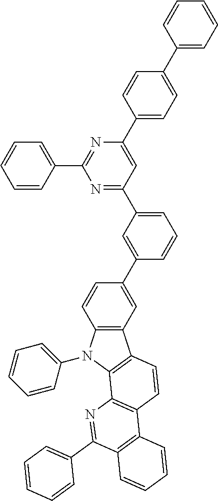 [SMILES: c1ccc(-c2ccc(-c3cc(-c4cccc(-c5ccc6c(c5)c5ccc7c8ccccc8c(-c8ccccc8)nc7c5n6-c5ccccc5)c4)nc(-c4ccccc4)n3)cc2)cc1] |

TABLE 5

| Com- | | Com- | |
|---|---|---|---|
| pound | FD-MS | pound | FD-MS |
| 1 | m/z = 727.27 | 2 | m/z = 803.30 |
| | C52H33N5 = 727.87 | | C58H37N5 = 803.97 |
| 3 | m/z = 803.30 | 4 | m/z = 777.29 |
| | C58H37N5 = 803.97 | | C56H35N5 = 777.93 |
| 5 | m/z = 777.29 | 6 | m/z = 726.28 |
| | C56H35N5 = 777.93 | | C53H34N4 = 826.88 |
| 7 | m/z = 802.31 | 8 | m/z = 802.31 |
| | C59H38N4 = 802.98 | | C59H38N4 = 802.98 |
| 9 | m/z = 776.29 | 10 | m/z = 776.29 |
| | C57H36N4 = 776.94 | | C57H36N4 = 776.94 |
| 11 | m/z = 727.27 | 12 | m/z = 803.30 |
| | C52H33N5 = 727.87 | | C58H37N5 = 803.97 |
| 13 | m/z = 803.30 | 14 | m/z = 777.29 |
| | C58H37N5 = 803.97 | | C56H35N5 = 777.93 |
| 15 | m/z = 777.29 | 16 | m/z = 726.28 |
| | C56H35N5 = 777.93 | | C53H34N4 = 826.88 |
| 17 | m/z = 802.31 | 18 | m/z = 802.31 |
| | C59H38N4 = 802.98 | | C59H38N4 = 802.98 |
| 19 | m/z = 776.29 | 20 | m/z = 776.29 |
| | C57H36N4 = 776.94 | | C57H36N4 = 776.94 |
| 21 | m/z = 688.26 | 22 | m/z = 738.28 |
| | C50H32N4 = 688.83 | | C54H34N4 = 738.89 |
| 23 | m/z = 738.28 | 24 | m/z = 840.33 |
| | C54H34N4 = 738.89 | | C62H40N4 = 841.03 |
| 25 | m/z = 788.29 | 26 | m/z = 838.31 |
| | C58H36N4 = 788.95 | | C62H38N4 = 839.01 |
| 27 | m/z = 812.29 | 28 | m/z = 764.29 |
| | C60H36N4 = 812.98 | | C56H36N4 = 764.93 |
| 29 | m/z = 700.26 | 30 | m/z = 750.28 |
| | C51H32N4 = 700.85 | | C5H34N4 = 750.91 |
| 31 | m/z = 750.28 | 32 | m/z = 776.29 |
| | C5H34N4 = 750.91 | | C57H36N4 = 776.94 |
| 33 | m/z = 852.33 | 34 | m/z = 800.29 |
| | C63H40N4 = 853.04 | | C59H36N4 = 800.97 |
| 35 | m/z = 850.31 | 36 | m/z = 824.29 |
| | C63H38N4 = 851.03 | | C61H36N4 = 824.99 |
| 37 | m/z = 674.25 | 38 | m/z = 750.28 |
| | C49H30N4 = 674.81 | | C55H34N4 = 750.91 |
| 39 | m/z = 674.25 | 40 | m/z = 750.28 |
| | C49H30N4 = 674.81 | | C55H34N4 = 750.91 |
| 41 | m/z = 688.26 | 42 | m/z = 738.28 |
| | C50H32N4 = 688.83 | | C54H34N4 = 738.89 |
| 43 | m/z = 738.28 | 44 | m/z = 840.33 |
| | C54H34N4 = 738.89 | | C62H40N4 = 841.03 |
| 45 | m/z = 788.29 | 46 | m/z = 838.31 |
| | C58H36N4 = 788.95 | | C62H38N4 = 839.01 |
| 47 | m/z = 812.29 | 48 | m/z = 764.29 |
| | C60H36N4 = 812.98 | | C56H36N4 = 764.93 |
| 49 | m/z = 700.26 | 50 | m/z = 750.28 |
| | C51H32N4 = 700.85 | | C55H34N4 = 750.91 |
| 51 | m/z = 750.28 | 52 | m/z = 776.29 |
| | C55H34N4 = 750.91 | | C57H36N4 = 776.94 |
| 53 | m/z = 852.33 | 54 | m/z = 800.29 |
| | C63H40N4 = 853.04 | | C59H36N4 = 800.97 |
| 55 | m/z = 850.31 | 56 | m/z = 824.29 |
| | C63H38N4 = 851.03 | | C61H36N4 = 824.99 |
| 57 | m/z = 727.27 | 58 | m/z = 803.30 |
| | C52H33N5 = 727.87 | | C58H37N5 = 803.97 |
| 59 | m/z = 803.30 | 60 | m/z = 777.29 |
| | C58H37N5 = 803.97 | | C56H35N5 = 777.93 |
| 61 | m/z = 777.29 | 62 | m/z = 726.28 |
| | C56H35N5 = 777.93 | | C53H34N4 = 726.88 |
| 63 | m/z = 802.31 | 64 | m/z = 802.31 |
| | C59H38N4 = 802.98 | | C59H38N4 = 802.98 |
| 65 | m/z = 776.29 | 66 | m/z = 776.29 |
| | C57H36N4 = 776.94 | | C57H36N4 = 776.94 |
| 67 | m/z = 727.27 | 68 | m/z = 803.30 |
| | C52H33N5 = 727.87 | | C58H37N5 = 803.97 |
| 69 | m/z = 803.30 | 70 | m/z = 777.29 |
| | C58H37N5 = 803.97 | | C56H35N5 = 777.93 |
| 71 | m/z = 777.29 | 72 | m/z = 726.28 |
| | C56H35N5 = 777.93 | | C53H34N4 = 726.88 |
| 73 | m/z = 802.31 | 74 | m/z = 802.31 |
| | C59H38N4 = 802.98 | | C59H38N4 = 802.98 |
| 75 | m/z = 776.29 | 76 | m/z = 776.29 |
| | C57H36N4 = 776.94 | | C57H36N4 = 776.94 |
| 77 | m/z = 688.26 | 78 | m/z = 738.28 |
| | C50H32N4 = 688.83 | | C54H34N4 = 738.89 |
| 79 | m/z = 738.28 | 80 | m/z = 840.33 |
| | C54H34N4 = 738.89 | | C62H40N4 = 841.03 |
| 81 | m/z = 788.29 | 82 | m/z = 838.31 |
| | C58H36N4 = 788.95 | | C62H38N4 = 839.01 |
| 83 | m/z = 812.29 | 84 | m/z = 764.29 |
| | C60H36N4 = 812.98 | | C56H36N4 = 764.93 |
| 85 | m/z = 700.26 | 86 | m/z = 750.28 |
| | C51H32N4 = 700.85 | | C55H34N4 = 750.91 |
| 87 | m/z = 750.28 | 88 | m/z = 776.29 |
| | C55H34N4 = 750.91 | | C57H36N4 = 776.94 |
| 89 | m/z = 852.33 | 90 | m/z = 800.29 |
| | C63H40N4 = 853.04 | | C59H36N4 = 800.97 |
| 91 | m/z = 850.31 | 92 | m/z = 824.29 |
| | C63H38N4 = 851.03 | | C61H36N4 = 824.99 |
| 93 | m/z = 674.25 | 94 | m/z = 750.28 |
| | C49H30N4 = 674.81 | | C55H34N4 = 750.91 |
| 95 | m/z = 674.25 | 96 | m/z = 750.28 |
| | C49H30N4 = 674.81 | | C55H34N4 = 750.91 |
| 97 | m/z = 688.26 | 98 | m/z = 738.28 |
| | C50H32N4 = 688.83 | | C54H34N4 = 738.89 |
| 99 | m/z = 738.28 | 100 | m/z = 840.33 |
| | C54H34N4 = 738.89 | | C62H40N4 = 841.03 |
| 101 | m/z = 788.29 | 102 | m/z = 838.31 |
| | C58H36N4 = 788.95 | | C62H38N4 = 839.01 |
| 103 | m/z = 812.29 | 104 | m/z = 764.29 |
| | C60H36N4 = 812.98 | | C56H36N4 = 764.93 |
| 105 | m/z = 700.26 | 106 | m/z = 750.28 |
| | C51H32N4 = 700.85 | | C55H34N4 = 750.91 |
| 107 | m/z = 750.28 | 108 | m/z = 776.29 |
| | C55H34N4 = 750.91 | | C57H36N4 = 776.94 |
| 109 | m/z = 852.33 | 110 | m/z = 800.29 |
| | C63H40N4 = 853.04 | | C59H36N4 = 800.97 |
| 111 | m/z = 850.31 | 112 | m/z = 824.29 |
| | C63H38N4 = 851.03 | | C61H36N4 = 824.99 |
| 113 | m/z = 727.27 | 114 | m/z = 803.30 |
| | C52H33N5 = 727.87 | | C58H37N5 = 803.97 |
| 115 | m/z = 803.30 | 116 | m/z = 777.29 |
| | C58H37N5 = 803.97 | | C56H35N5 = 777.93 |
| 117 | m/z = 777.29 | 118 | m/z = 726.28 |
| | C56H35N5 = 777.93 | | C53H34N4 = 726.88 |
| 119 | m/z = 802.31 | 120 | m/z = 802.31 |
| | C59H38N4 = 802.98 | | C59H38N4 = 802.98 |
| 121 | m/z = 776.29 | 122 | m/z = 776.29 |
| | C57H36N4 = 776.94 | | C57H36N4 = 776.94 |
| 123 | m/z = 727.27 | 124 | m/z = 803.30 |
| | C52H33N5 = 727.87 | | C58H37N5 = 803.97 |
| 125 | m/z = 803.30 | 126 | m/z = 777.29 |
| | C58H37N5 = 803.97 | | C56H35N5 = 777.93 |
| 127 | m/z = 777.29 | 128 | m/z = 726.28 |
| | C56H35N5 = 777.93 | | C53H34N4 = 726.88 |
| 129 | m/z = 802.31 | 130 | m/z = 802.31 |
| | C59H38N4 = 802.98 | | C59H38N4 = 802.98 |
| 131 | m/z = 776.29 | 132 | m/z = 776.29 |
| | C57H36N4 = 776.94 | | C57H36N4 = 776.94 |
| 133 | m/z = 688.26 | 134 | m/z = 738.28 |
| | C50H32N4 = 688.83 | | C54H34N4 = 738.89 |
| 135 | m/z = 738.28 | 136 | m/z = 840.33 |
| | C54H34N4 = 738.89 | | C62H40N4 = 841.03 |
| 137 | m/z = 788.29 | 138 | m/z = 838.31 |
| | C58H36N4 = 788.95 | | C62H38N4 = 839.01 |
| 139 | m/z = 812.29 | 140 | m/z = 764.29 |
| | C60H36N4 = 812.98 | | C56H36N4 = 764.93 |
| 141 | m/z = 700.26 | 142 | m/z = 750.28 |
| | C51H32N4 = 700.85 | | C55H34N4 = 750.91 |
| 143 | m/z = 750.28 | 144 | m/z = 776.29 |
| | C55H34N4 = 750.91 | | C57H36N4 = 776.94 |
| 145 | m/z = 852.33 | 146 | m/z = 800.29 |
| | C63H40N4 = 853.04 | | C59H36N4 = 800.97 |
| 147 | m/z = 850.31 | 148 | m/z = 824.29 |
| | C63H38N4 = 851.03 | | C61H36N4 = 824.99 |
| 149 | m/z = 674.25 | 150 | m/z = 750.28 |
| | C49H30N4 = 674.81 | | C55H34N4 = 750.91 |
| 151 | m/z = 674.25 | 152 | m/z = 750.28 |
| | C49H30N4 = 674.81 | | C55H34N4 = 750.91 |
| 153 | m/z = 688.26 | 154 | m/z = 738.28 |
| | C50H32N4 = 688.83 | | C54H34N4 = 738.89 |
| 155 | m/z = 738.28 | 156 | m/z = 840.33 |
| | C54H34N4 = 738.89 | | C62H40N4 = 841.03 |
| 157 | m/z = 788.29 | 158 | m/z = 838.31 |
| | C58H36N4 = 788.95 | | C62H38N4 = 739.01 |
| 159 | m/z = 812.29 | 160 | m/z = 764.29 |
| | C60H36N4 = 812.98 | | C56H36N4 = 764.93 |
| 161 | m/z = 700.26 | 162 | m/z = 750.28 |
| | C51H32N4 = 700.85 | | C55H34N4 = 750.91 |
| 163 | m/z = 750.28 | 164 | m/z = 776.29 |
| | C55H34N4 = 750.91 | | C57H36N4 = 776.94 |
| 165 | m/z = 852.33 | 166 | m/z = 800.29 |
| | C63H40N4 = 853.04 | | C59H36N4 = 800.97 |
| 167 | m/z = 850.31 | 168 | m/z = 824.29 |
| | C63H38N4 = 851.03 | | C61H36N4 = 824.99 |
| 169 | m/z = 727.27 | 170 | m/z = 803.30 |
| | C52H33N5 = 727.87 | | C58H37N5 = 803.97 |
| 171 | m/z = 803.30 | 172 | m/z = 777.29 |
| | C58H37N5 = 803.97 | | C56H35N5 = 777.93 |
| 173 | m/z = 777.29 | 174 | m/z = 726.28 |
| | C56H35N5 = 777.93 | | C53H34N4 = 726.88 |
| 175 | m/z = 802.31 | 176 | m/z = 802.31 |
| | C59H38N4 = 802.98 | | C59H38N4 = 802.98 |
| 177 | m/z = 776.29 | 178 | m/z = 776.29 |
| | C57H36N4 = 776.94 | | C57H36N4 = 776.94 |
| 179 | m/z = 727.27 | 180 | m/z = 803.30 |
| | C52H33N5 = 727.87 | | C58H37N5 = 803.97 |
| 181 | m/z = 803.30 | 182 | m/z = 777.29 |
| | C58H37N5 = 803.97 | | C56H35N5 = 777.93 |
| 183 | m/z = 777.29 | 184 | m/z = 726.28 |
| | C56H35N5 = 777.93 | | C53H34N4 = 726.88 |
| 185 | m/z = 802.31 | 186 | m/z = 802.31 |
| | C59H38N4 = 802.98 | | C59H38N4 = 802.98 |
| 187 | m/z = 776.29 | 188 | m/z = 776.29 |
| | C57H36N4 = 776.94 | | C57H36N4 = 776.94 |
| 189 | m/z = 688.26 | 190 | m/z = 738.28 |
| | C50H32N4 = 688.83 | | C54H34N4 = 738.89 |
| 191 | m/z = 738.28 | 192 | m/z = 840.33 |
| | C54H34N4 = 738.89 | | C62H40N4 = 841.03 |
| 193 | m/z = 788.29 | 194 | m/z = 838.31 |
| | C58H36N4 = 788.95 | | C62H38N4 = 839.01 |
| 195 | m/z = 812.29 | 196 | m/z = 764.29 |
| | C60H36N4 = 812.98 | | C56H36N4 = 764.93 |
| 197 | m/z = 700.26 | 198 | m/z = 750.28 |
| | C51H32N4 = 700.85 | | C55H34N4 = 750.91 |
| 199 | m/z = 750.28 | 200 | m/z = 776.29 |
| | C55H34N4 = 750.91 | | C57H36N4 = 776.94 |
| 201 | m/z = 852.33 | 202 | m/z = 800.29 |
| | C63H40N4 = 853.04 | | C59H36N4 = 800.97 |
| 203 | m/z = 850.31 | 204 | m/z = 824.29 |
| | C63H38N4 = 851.03 | | C61H36N4 = 824.99 |
| 205 | m/z = 674.25 | 206 | m/z = 750.28 |
| | C49H30N4 = 674.81 | | C55H34N4 = 750.91 |
| 207 | m/z = 674.25 | 208 | m/z = 750.28 |
| | C49H30N4 = 674.81 | | C55H34N4 = 750.91 |
| 209 | m/z = 688.26 | 210 | m/z = 738.28 |
| | C50H32N4 = 688.83 | | C54H34N4 = 738.89 |
| 211 | m/z = 738.28 | 212 | m/z = 840.33 |
| | C54H34N4 = 738.89 | | C62H40N4 = 841.03 |
| 213 | m/z = 788.29 | 214 | m/z = 838.31 |
| | C58H36N4 = 788.95 | | C62H38N4 = 839.01 |
| 215 | m/z = 812.29 | 216 | m/z = 764.29 |
| | C60H36N4 = 812.98 | | C56H36N4 = 764.93 |
| 217 | m/z = 700.26 | 218 | m/z = 750.28 |
| | C51H32N4 = 700.85 | | C55H34N4 = 750.91 |
| 219 | m/z = 750.28 | 220 | m/z = 776.29 |
| | C55H34N4 = 750.91 | | C57H36N4 = 776.94 |
| 221 | m/z = 852.33 | 222 | m/z = 800.29 |
| | C63H40N4 = 853.04 | | C59H36N4 = 800.97 |
| 223 | m/z = 850.31 | 224 | m/z = 824.29 |
| | C63H38N4 = 851.03 | | C61H36N4 = 824.99 |

### Preparation Example: Manufacture of Organic Light Emitting Diode

The abbreviations used in the Examples below are as follows.

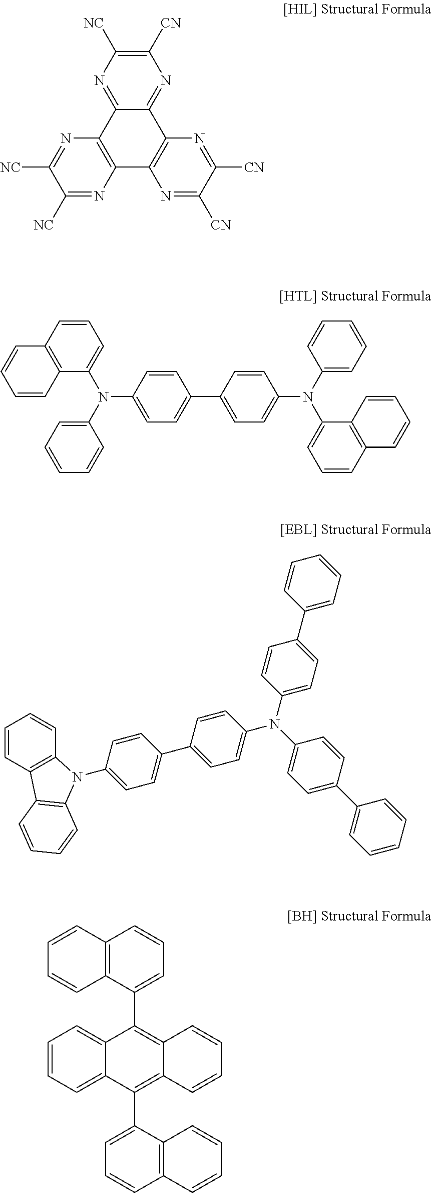

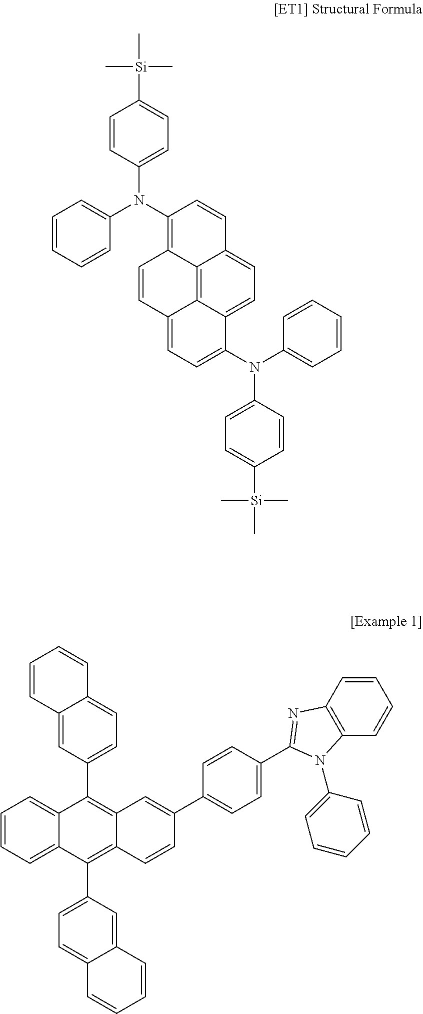 [SMILES: C[Si](C)(C)c1ccc(N(c2ccccc2)c2ccc3ccc4c(N(c5ccccc5)c5ccc([Si](C)(C)C)cc5)ccc5ccc2c3c54)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

A substrate used for manufacturing a diode was ultrasonically cleaned with distilled water for 10 minutes, dried in an oven at 100° C. for 30 minutes, and transferred to a vacuum deposition chamber.

The substrate used in the present invention was a top emission type, and an anode was formed with a metal/indium tin oxide (ITO) layer. Examples of metal materials which may be used here include Ag, Au, Pt, Al, Cu, Ni, Mo, Cr, or alloys thereof, and the like. The indium tin oxide (ITO) may be stacked with a thickness between 7 nm and 15 nm. On the ITO electrode, a hole injecting layer, a hole transport layer, an electron blocking layer, an emitting layer, an electron transport layer, and an electron injecting layer are formed sequentially. The hole injecting layer (HIL) was deposited at a thickness of 10 nm and added with about 3% dopant to facilitate good hole injection. The hole transport layer (HTL) was deposited at a thickness of 120 nm. On the deposited HIL, the electron blocking layer (EBL) was deposited at a thickness of 15 nm. Then, the organic emitting layer (OEL) was deposited at a thickness of 20 nm and added with 5% of dopant. Next, on the organic emitting layer, Compound 118 synthesized in Preparation Example 1 and lithium quinolate (LiQ) were formed as the electron transport layer at a weight ratio of 2:1 and deposited at a thickness of 30 nm. In this process, the deposition rate of the organic material was maintained at 0.5 to 1.0 Å/sec, and the vacuum pressure at the time of deposition was maintained at 1×10⁻⁷ to 4×10⁻⁷ torr. To form a resonance structure, the total thickness of the organic material varies according to the light emission color. Further, in order to maximize the resonance effect, the electrode was formed as a semi-transparent electrode. The metal used for the semi-transparent electrode may be Al, Mg, Ag, LiF, or an alloy thereof, and they should be applied to have a ratio and a specific thickness so as to induce light reflection characteristics. The thickness of a cathode used here was 14 nm. Finally, a light-efficiency improvement layer (capping layer) was deposited at a thickness of 63 nm. After vacuum deposition, the substrate was transferred to a glove box and subjected to a sealing process. The sealing member may be provided with a glass cap including a getter therein, and may be subjected to UV irradiation (curing) after applying a resin for sealing, and thus oxygen and moisture may be blocked from penetrating the deposition surface. For reference, % means a percent by weight unless otherwise defined in the present Example.

### Examples 2 to 10 and Comparative Example 1

Examples 2 to 10 and Comparative Example 1 were prepared in the same manner as in Example 1, except that the compounds described in Table 6 below were used as the electron transport layer instead of using Compound 118.

### Test Example

For the organic light emitting diodes manufactured in Examples 1 to 10 and Comparative Example 1, the driving voltage and current efficiency were measured at a current density of 10 mA/cm2, that is, light emission efficiency was measured, and the time to reach 95% as compared to the initial luminance of 1000 cd/m² (LT95) was also measured, the results of which are shown in Table 6 below.

TABLE 6

| | | Driving | Current | |
|---|---|---|---|---|
| | | Voltage | Efficiency | Life Time 95 at |
| | Compound | (V) | (cd/A) | 1000 cd/m² |
| Example 1 | 118 | 4.13 | 7.03 | 141 |
| Example 2 | 119 | 4.19 | 6.99 | 146 |
| Example 3 | 6 | 4.09 | 7.03 | 149 |
| Example 4 | 7 | 4.17 | 7.01 | 143 |
| Example 5 | 174 | 4.13 | 7.29 | 159 |
| Example 6 | 175 | 4.38 | 7.21 | 169 |
| Example 7 | 57 | 4.03 | 6.81 | 153 |
| Example 8 | 62 | 4.26 | 7.23 | 171 |
| Example 9 | 63 | 4.29 | 7.18 | 164 |
| Example 10 | 67 | 4.25 | 7.17 | 183 |
| Comparative | ET1 | 4.56 | 6.63 | 101 |
| Example 1 | | | | |

As can be seen from Table 6, the organic light emitting diodes manufactured using the compound of the present invention as a material for the electron transport layer exhibited excellent characteristics in view of efficiency and stability as compared with the case of using the compound of Comparative Example 1.

According to the present invention, there are provided organic compounds capable of improving the light emitting efficiency, stability, and lifetime of an organic light emitting diode, an organic light emitting diode including the same, and a display including the organic light emitting diode.

While the present invention is described with reference to preferable embodiments thereof, the present invention is not limited to these embodiments, and various modifications can be made within the scope of the appended claims and the detailed description of the present invention.

What is claimed is:

1. An organic compound represented by Chemical Formula 1 below: 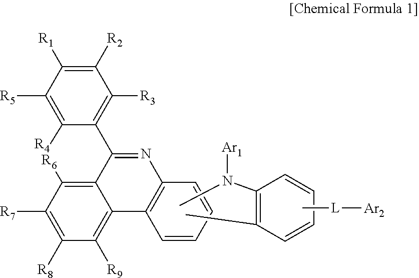
In Chemical Formula 1,
R₁ to R₉ are each, independently, any one selected from the group consisting of hydrogen, a halogen, a nitro group, a substituted or unsubstituted C1-C6 alkyl group, a substituted or unsubstituted C3-C6 cycloalkyl group, a substituted or unsubstituted C6-C40 aryl group, and a substituted or unsubstituted C5-C40 heteroaryl group,
L represents a single bond, and is any one selected from the group consisting of an alkylene group, a cycloalkylene group, a heterocycloalkylene group, an arylene group, and a heteroarylene group, and
Ar₁ and Ar₂ are each, independently, any one selected from the group consisting of hydrogen, a substituted or unsubstituted C3-C6 cycloalkyl group, a substituted or unsubstituted C3-C6 heterocycloalkyl group, a substituted or unsubstituted C6-C40 aryl group, and a substituted or unsubstituted C2-C40 heteroaryl group.

2. The organic compound of claim 1, wherein the Ar₁ is any one selected from the group consisting of a phenyl group, a biphenyl group, a terphenyl group, a naphthyl group, an anthracenyl group, a phenanthrene group, a pyrenyl group, a triphenylene group, a perylenyl group, a chrysenyl group, a carbazole group, a thiophene group, a furan group, a pyrrolyl group, an imidazole group, a thiazole group, an oxazole group, an oxadiazole group, a triazole group, a pyridyl group, a pyradazine group, a quinolinyl group, an isoquinoline group, and an acridyl group.

3. The organic compound of claim 1, wherein the Ar₂ is a pyridyl group, a quinolinyl group, an isoquinolinyl group, a benzoquinolinyl group, a quinoxanyl group, a naphthyridyl group, a pyrazinyl group, a pyrimidyl group, a pyridazinyl group, a phenanthrolyl group, an imidazopyridyl group, a triazyl group, an acridinyl group, an imidazolyl group, a benzimidazolyl group, an oxazolyl group, a benzoxazolyl group, a thiazolyl group, or a benzothiazolyl group, or 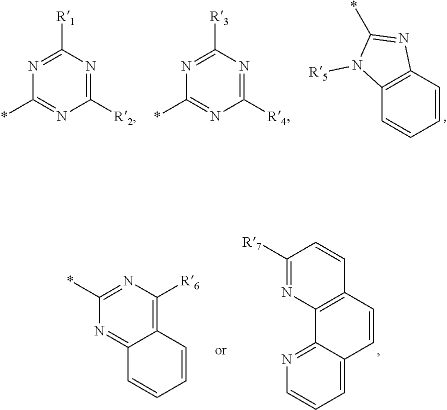
wherein R′₁ to R′₇ are each, independently, any one selected from the group consisting of hydrogen, a halogen, CN, Si(CH)₃, CF₃, nitro, a substituted or unsubstituted C1-C10 alkyl group, a substituted or unsubstituted C3-C10 cycloalkyl group, a substituted or unsubstituted C1-C10 alkoxy group, a phenyl group, a biphenyl group, a terphenyl group, a naphthyl group, an anthracenyl group, a phenanthrene group, a pyrenyl group, a triphenylene group, a perylenyl group, a chrysenyl group, a carbazole group, a thiophene group, a furan group, a pyrrolyl group, an imidazole group, a thiazole group, an oxazole group, an oxadiazole group, a triazole group, a pyridyl group, a pyradazine group, a quinolinyl group, an isoquinoline group, and an acridyl group.

4. The organic compound of claim 1, wherein the organic compound is represented by Structural Formulas below.

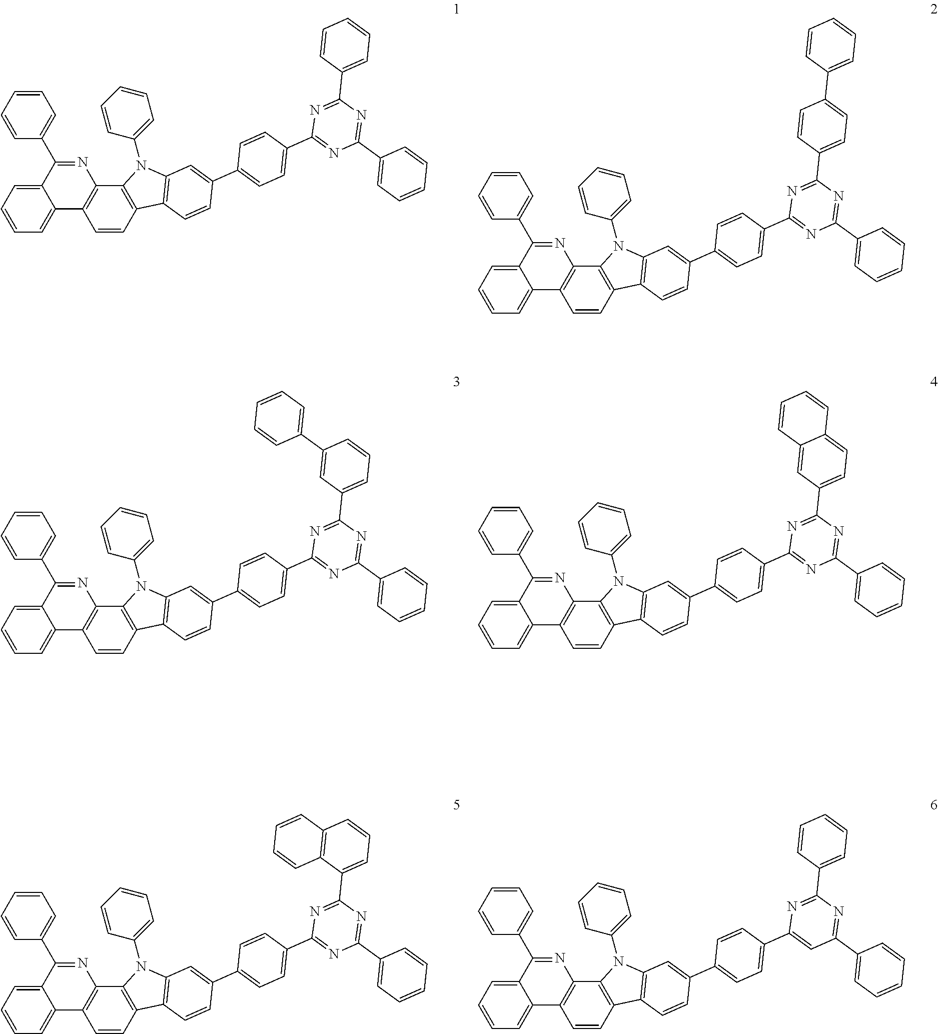

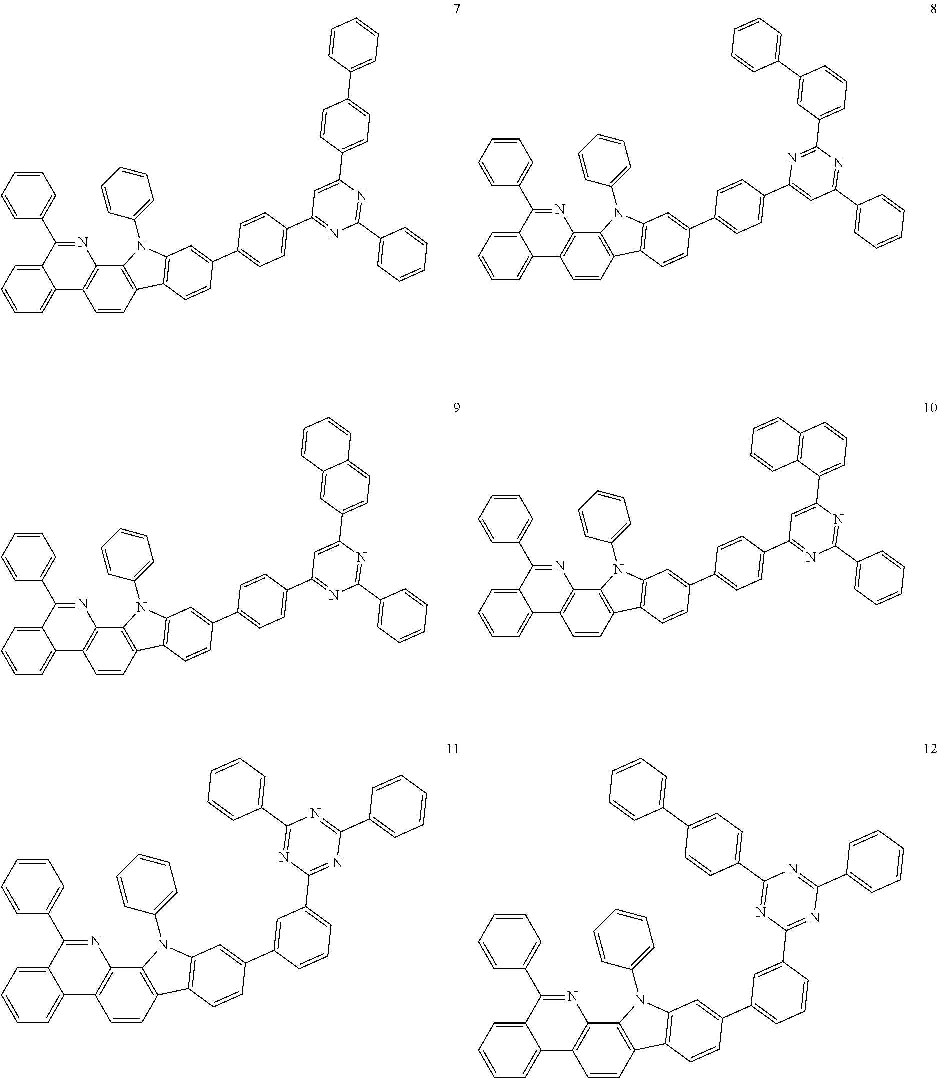

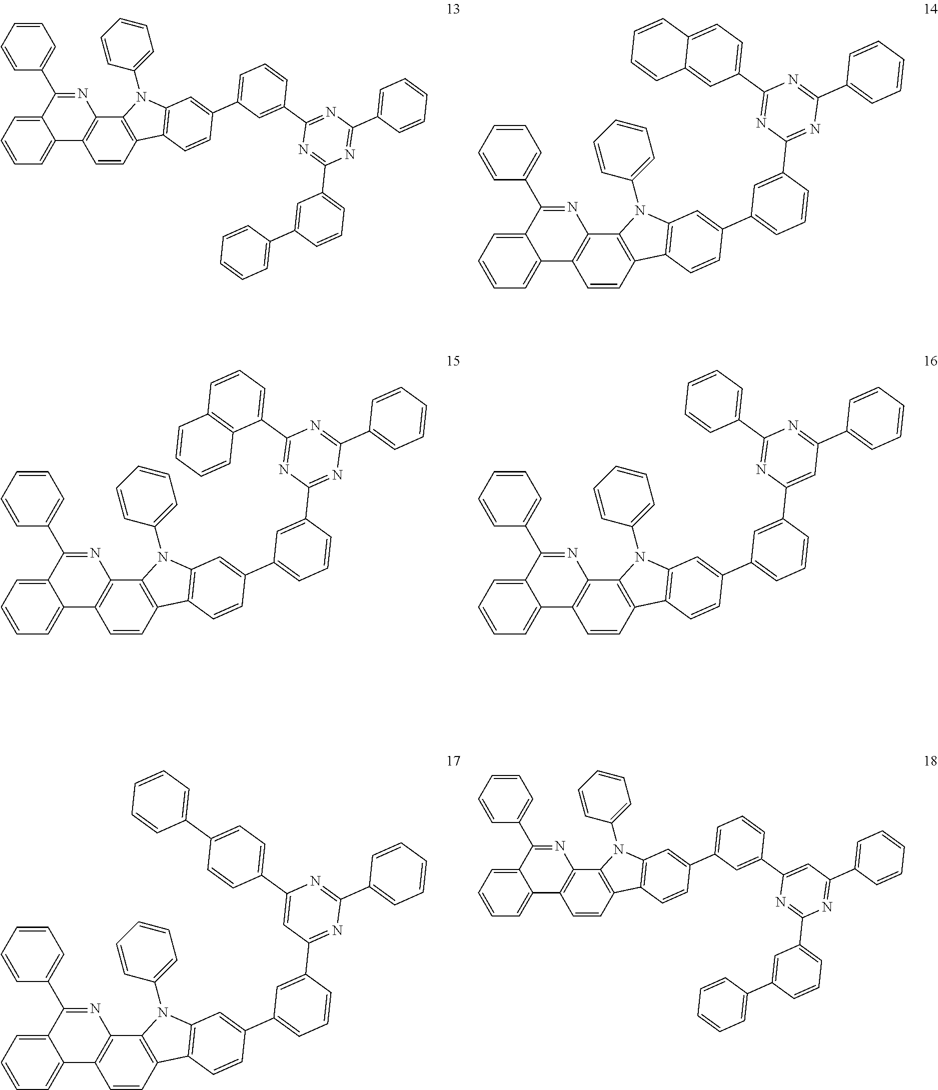

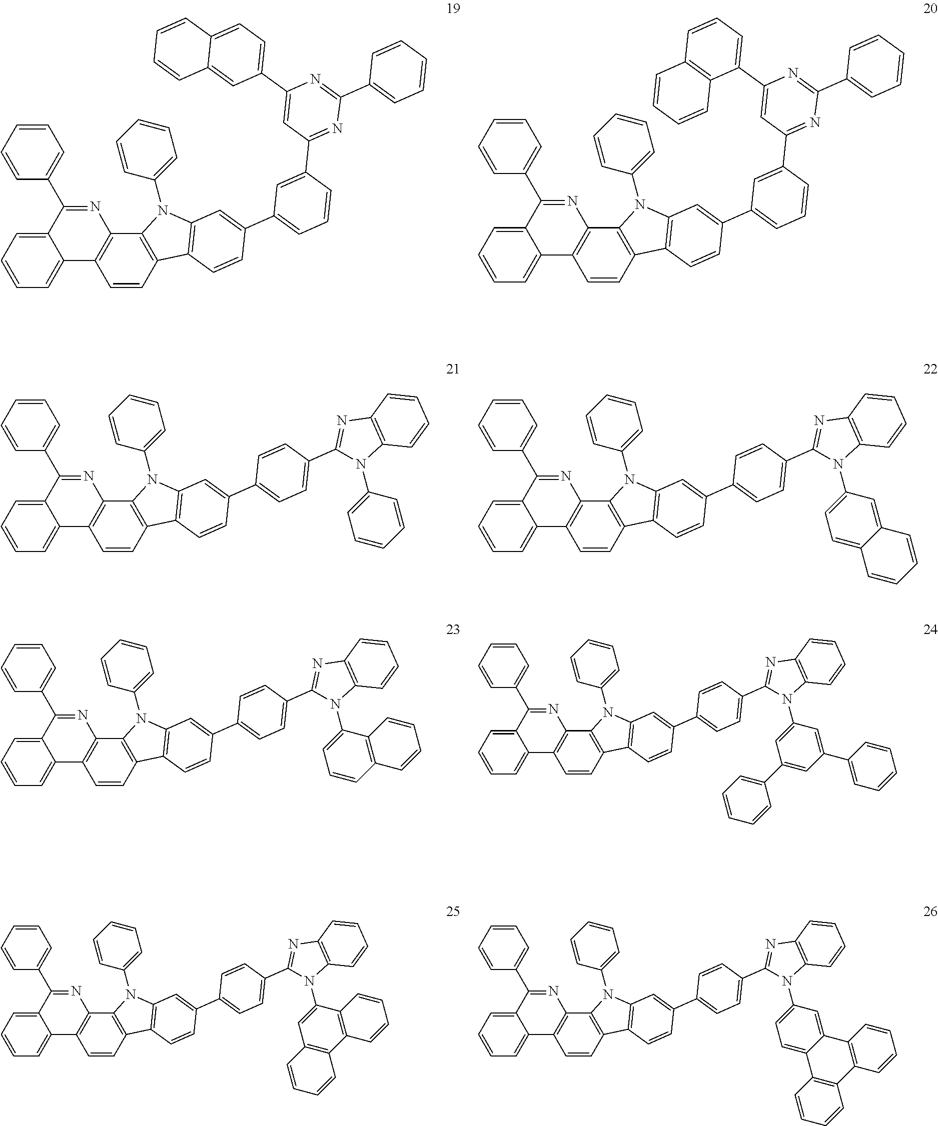

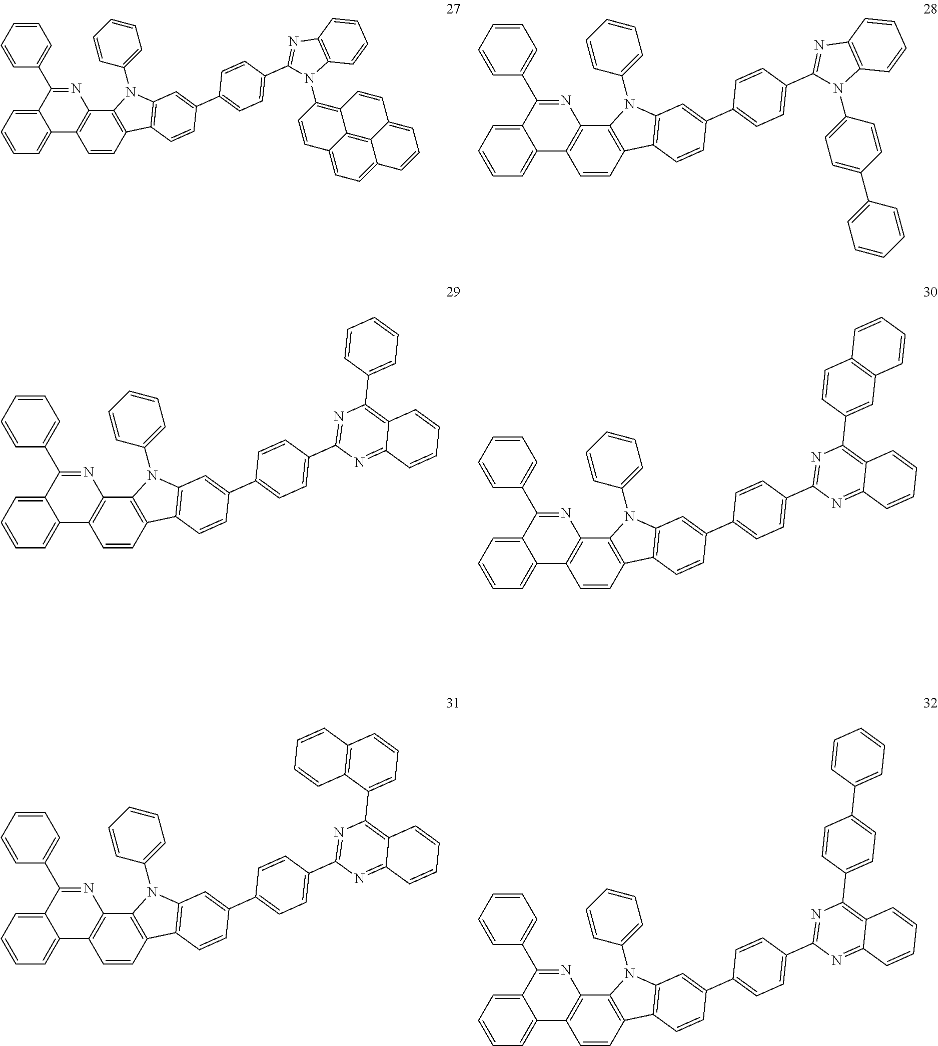

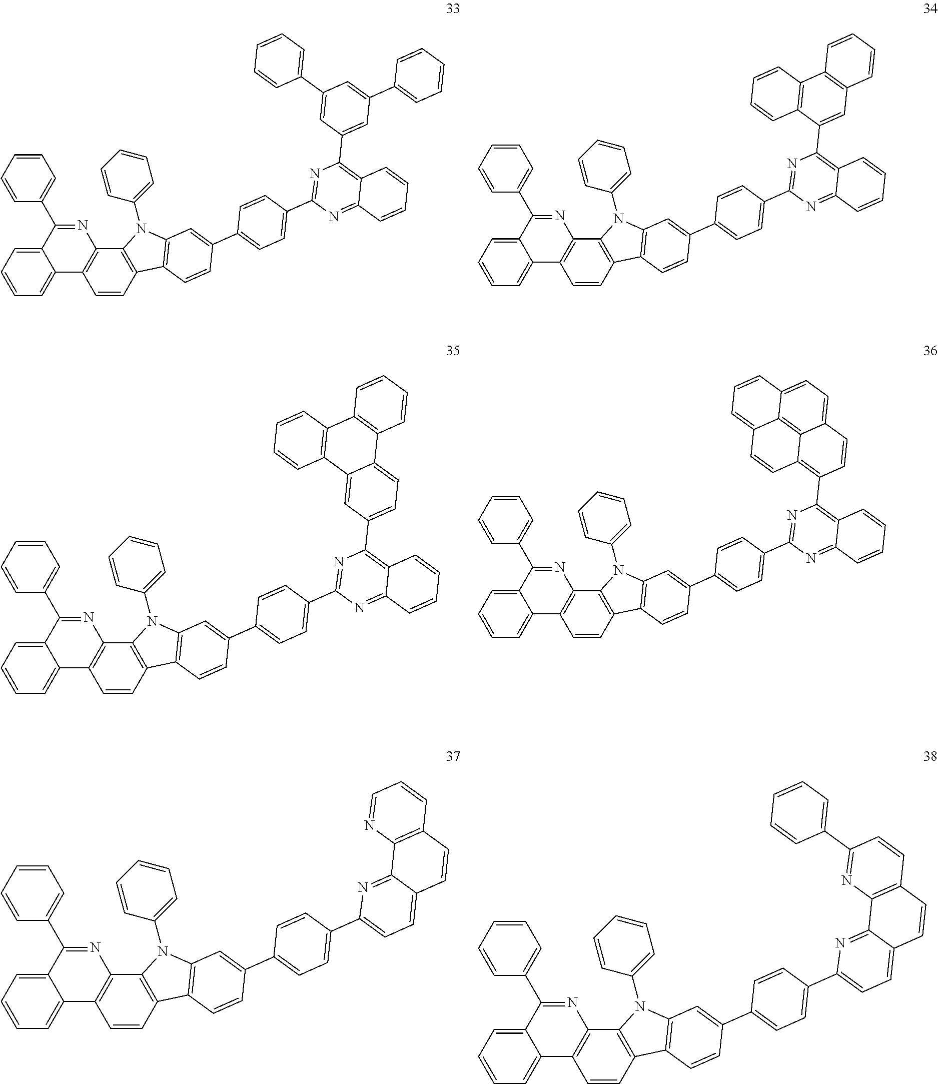

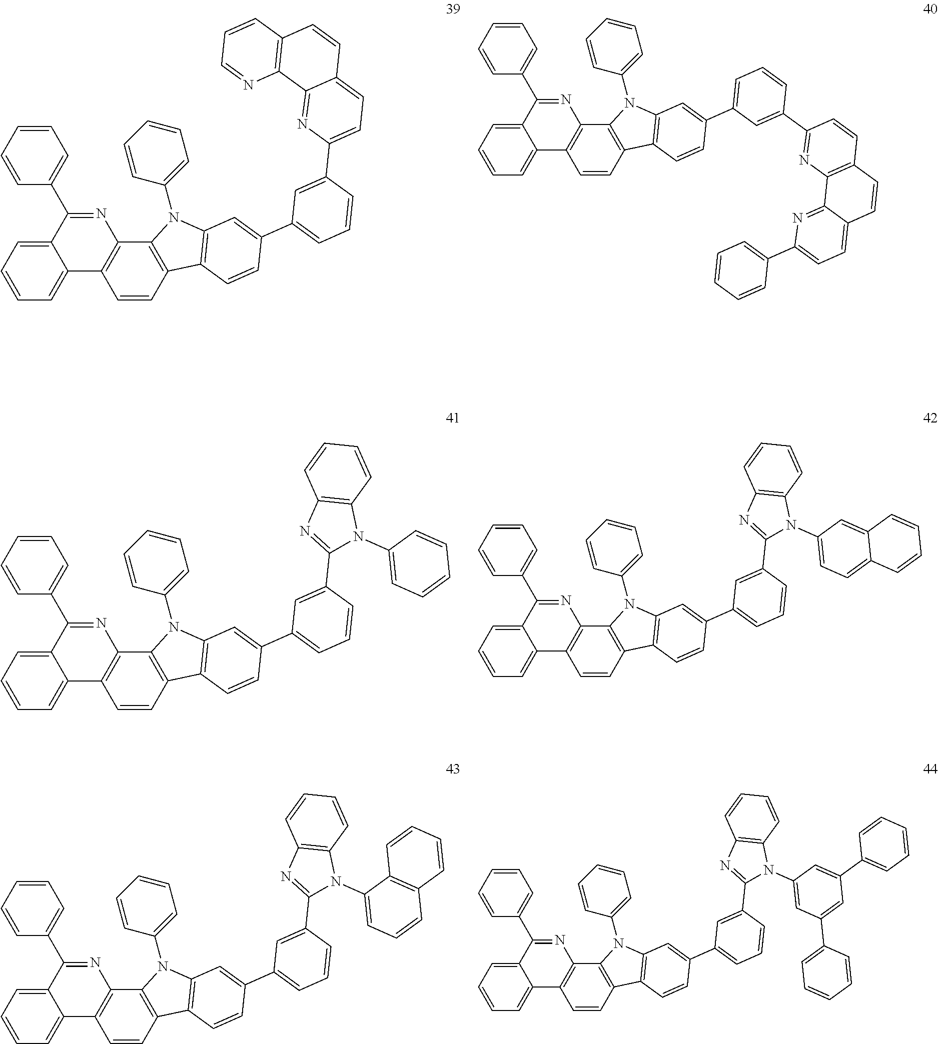

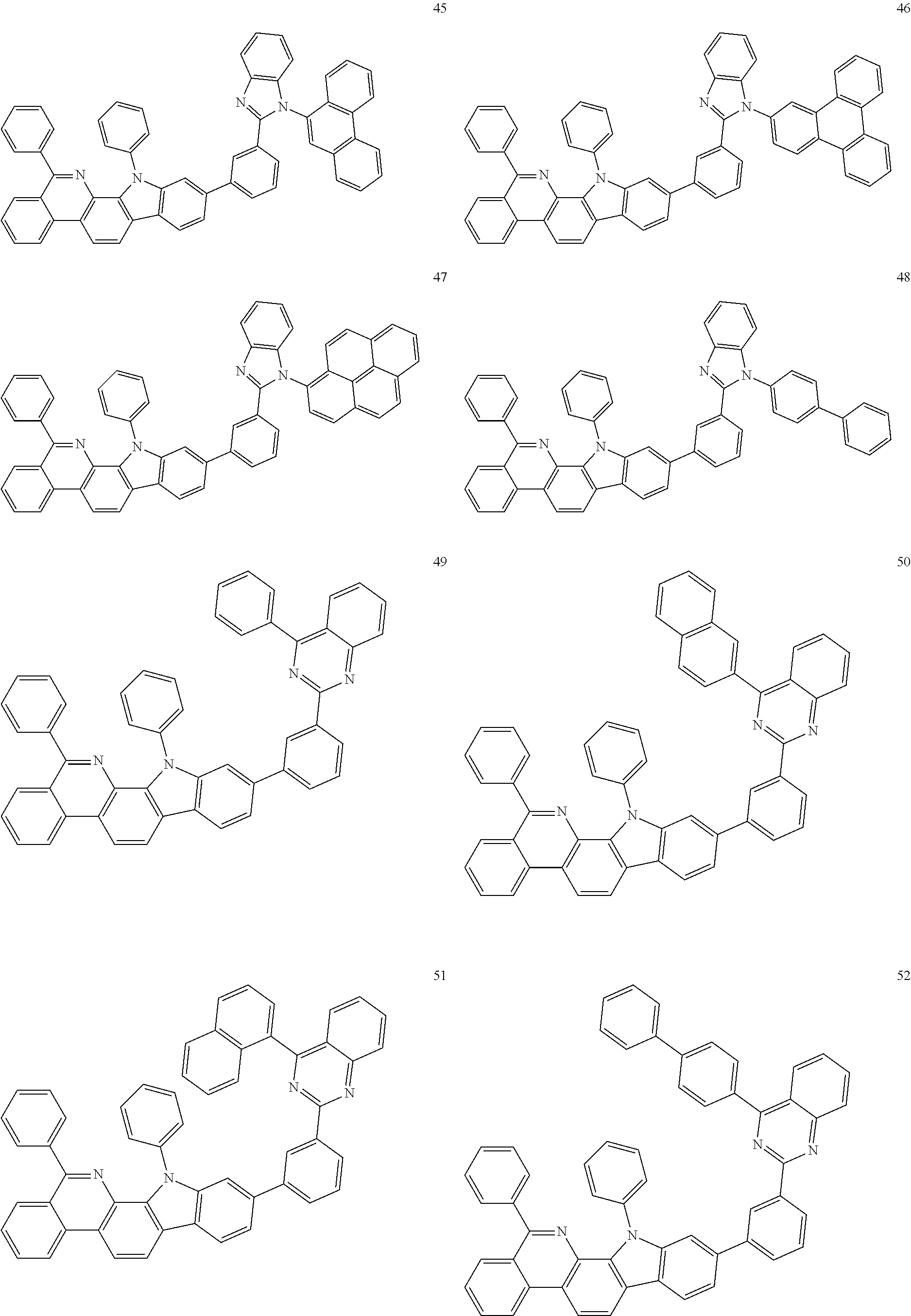

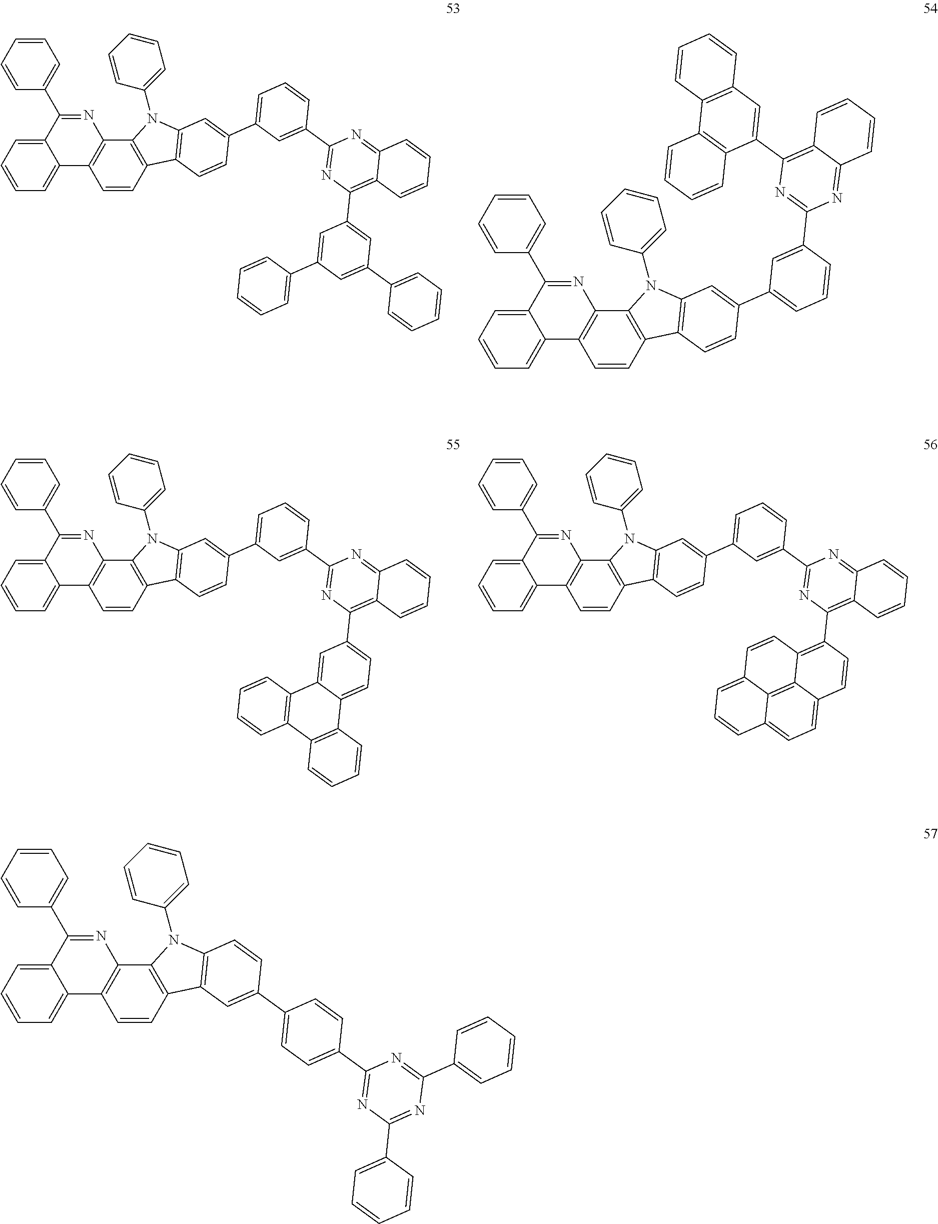

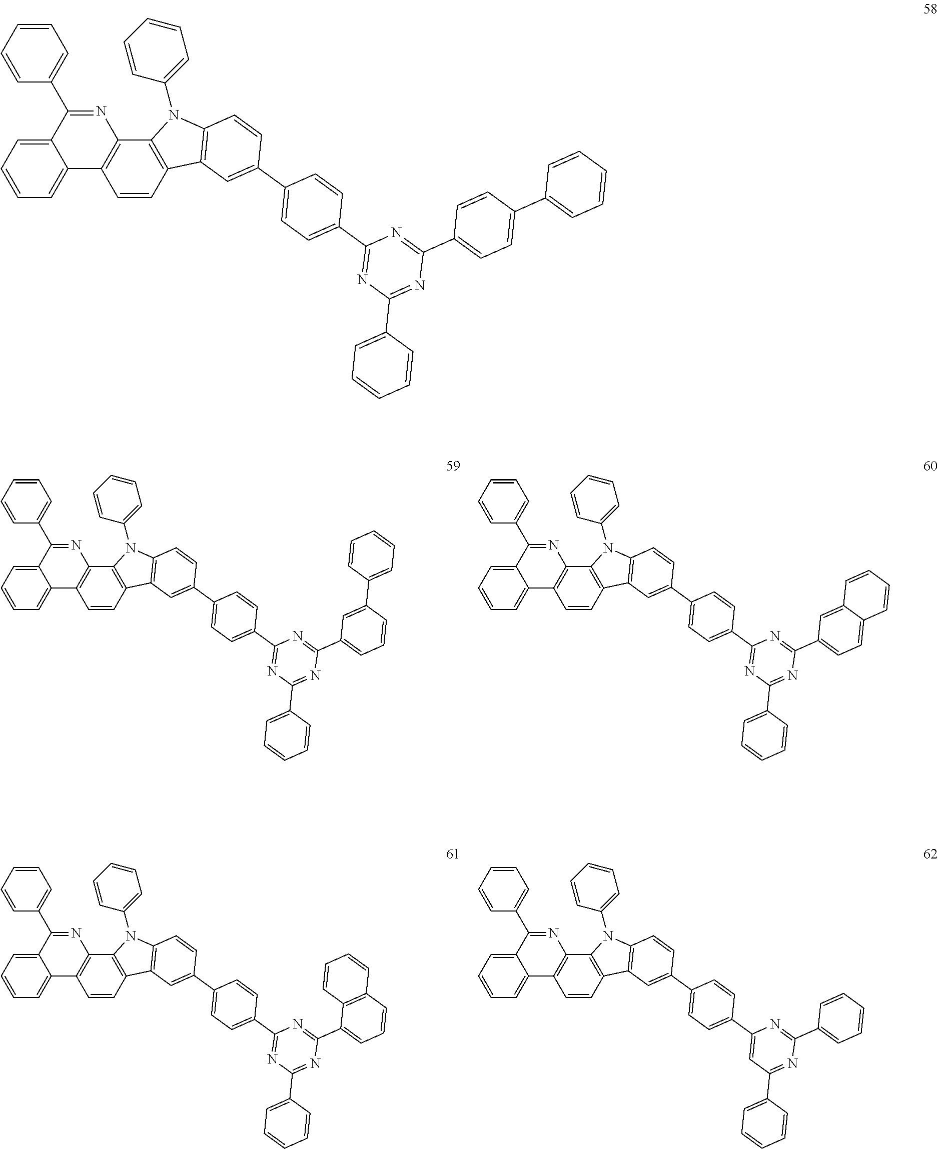

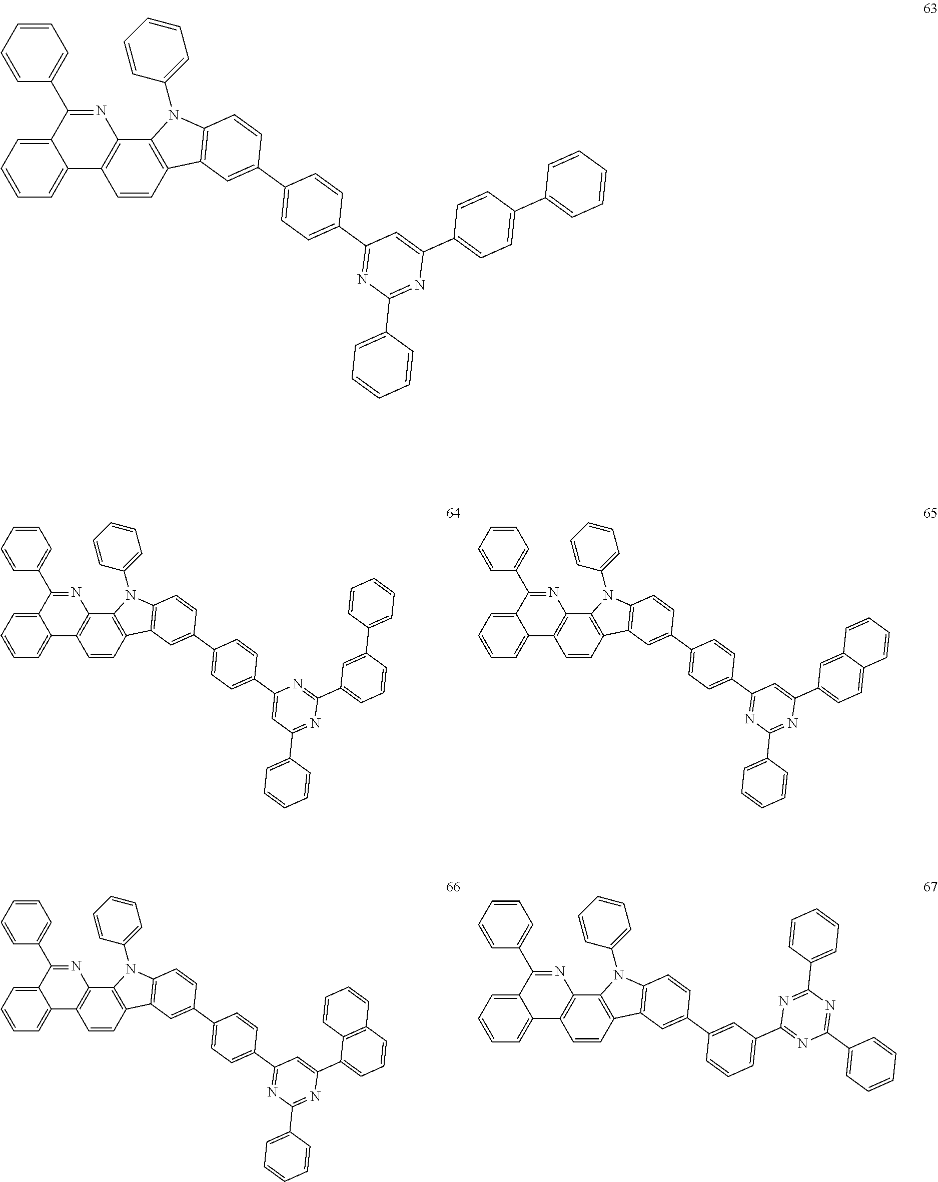

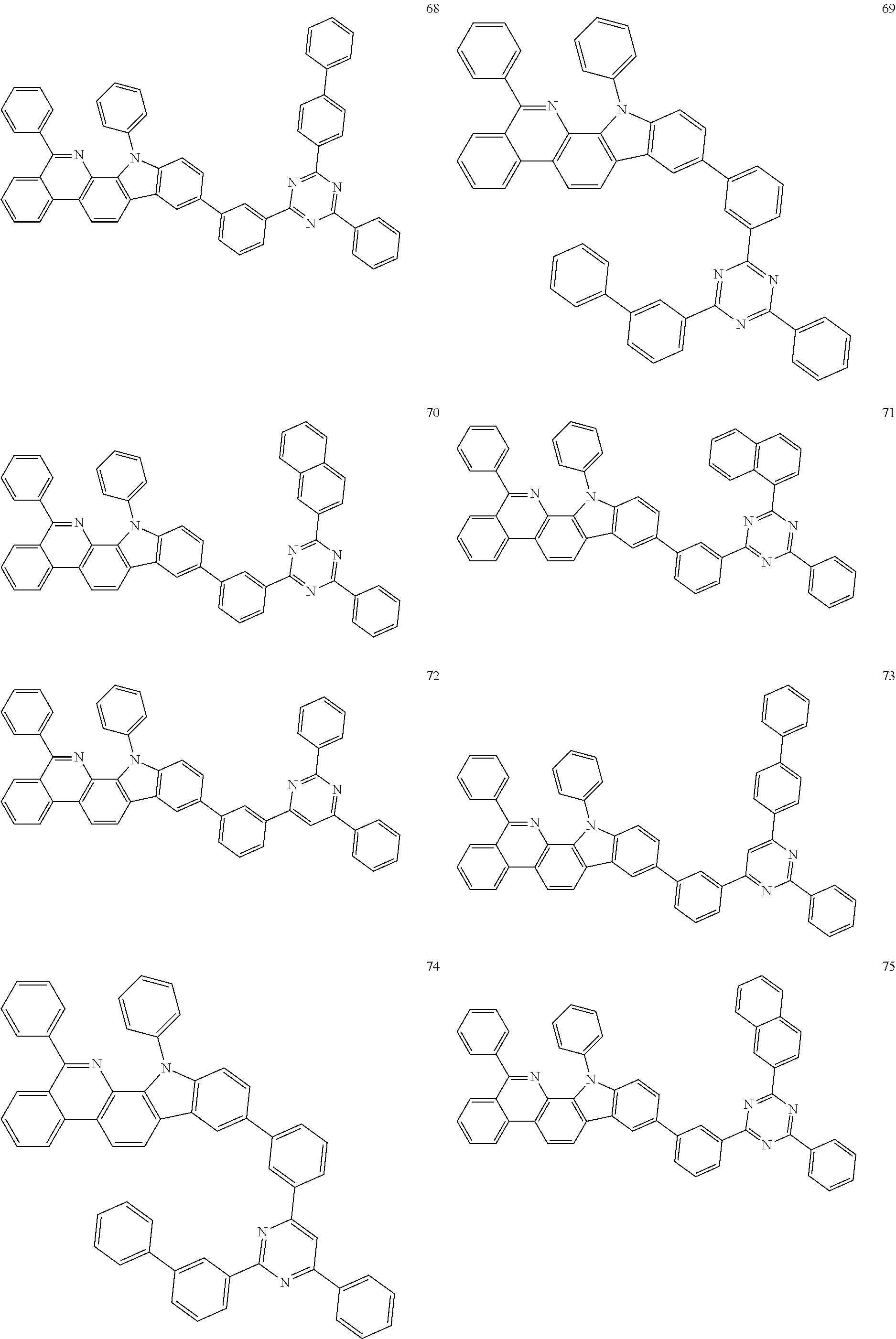

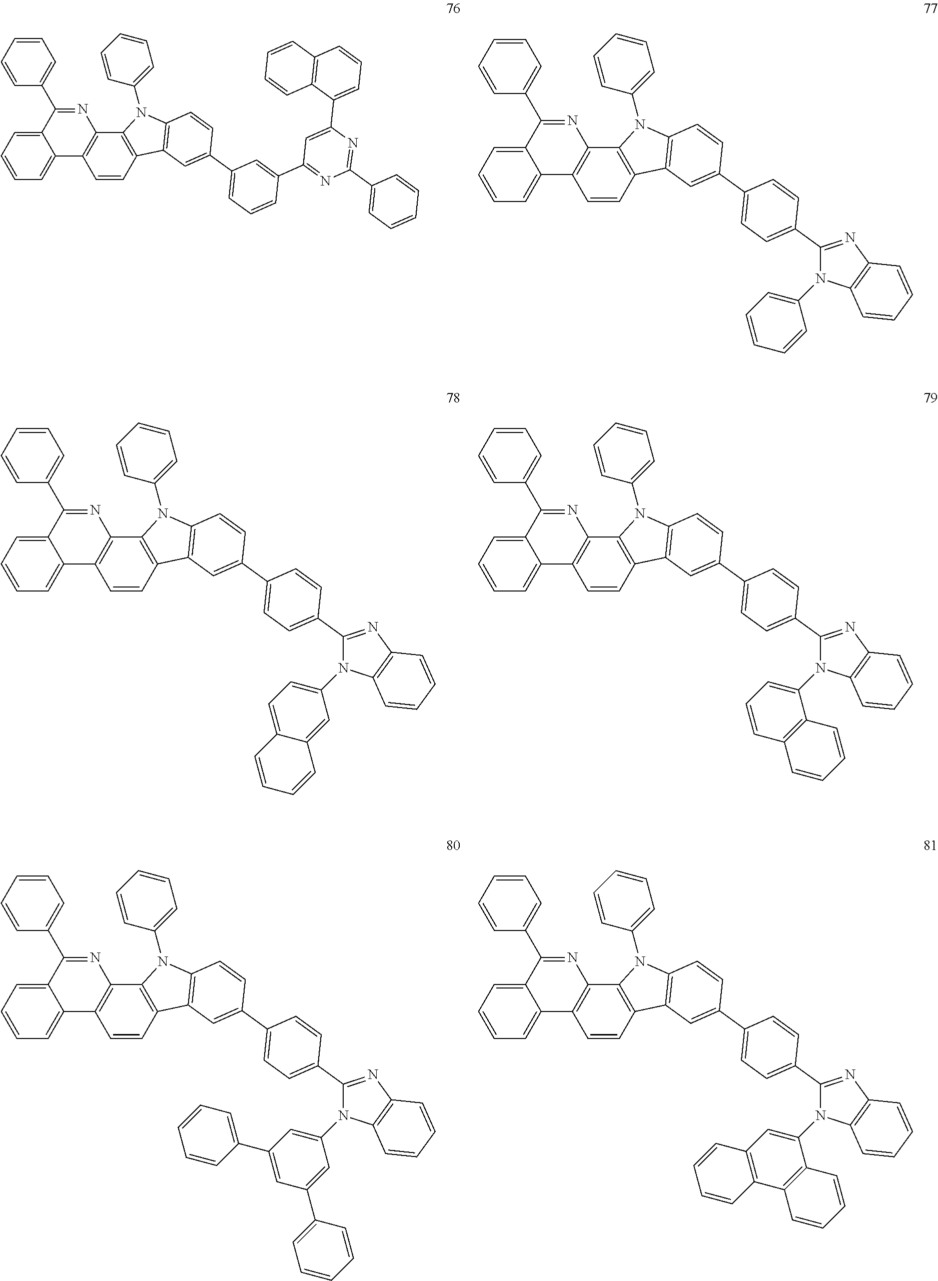

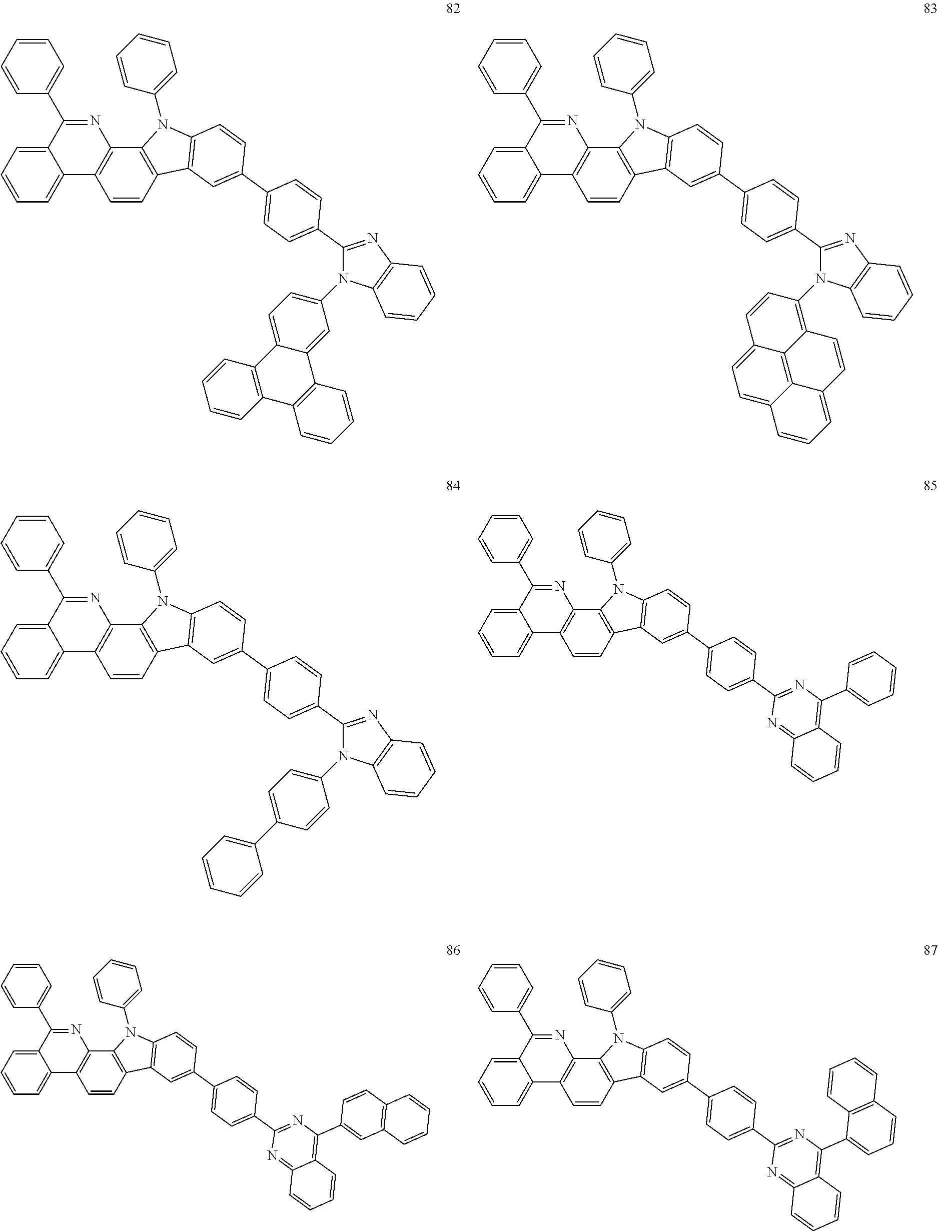

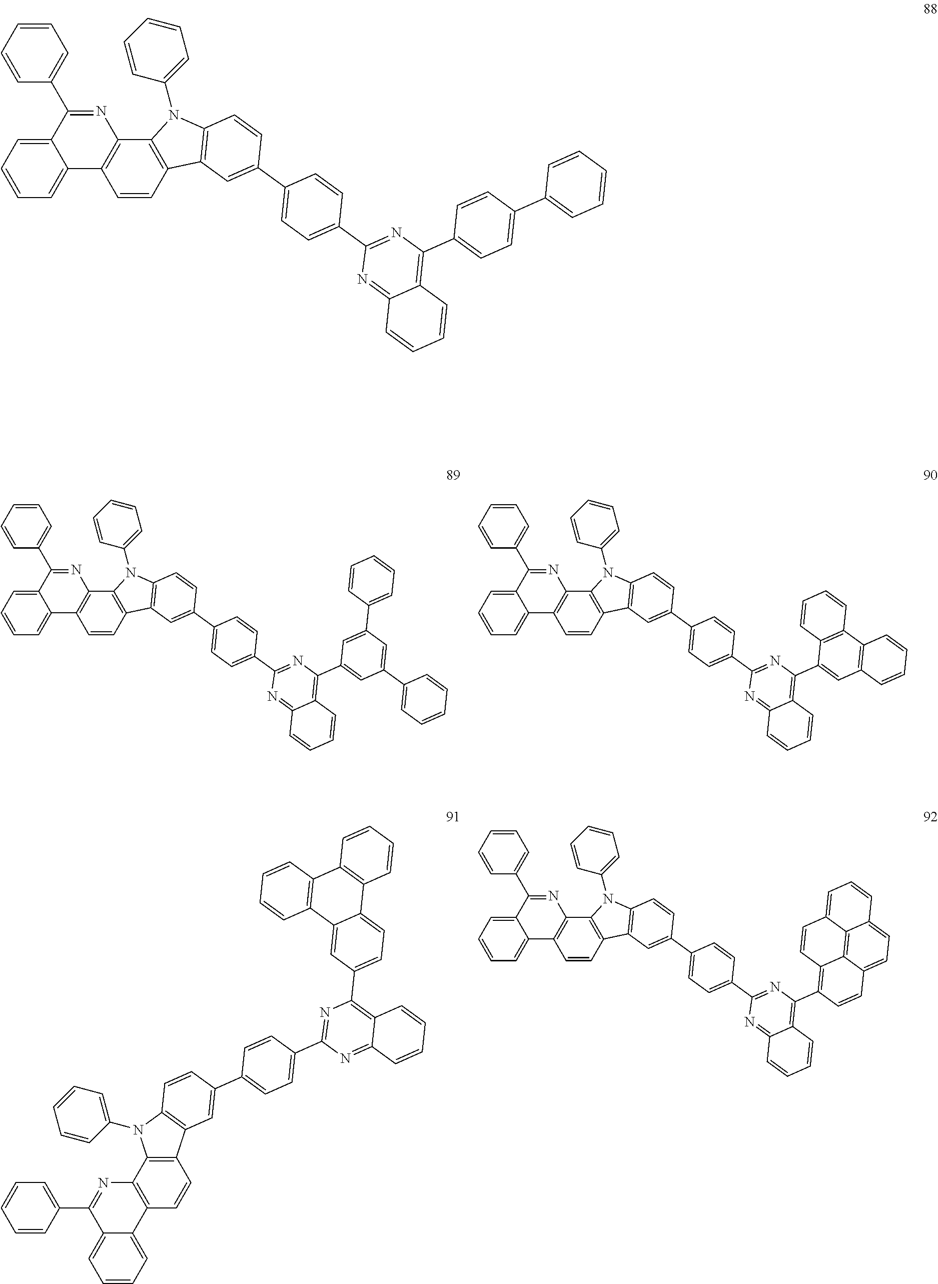

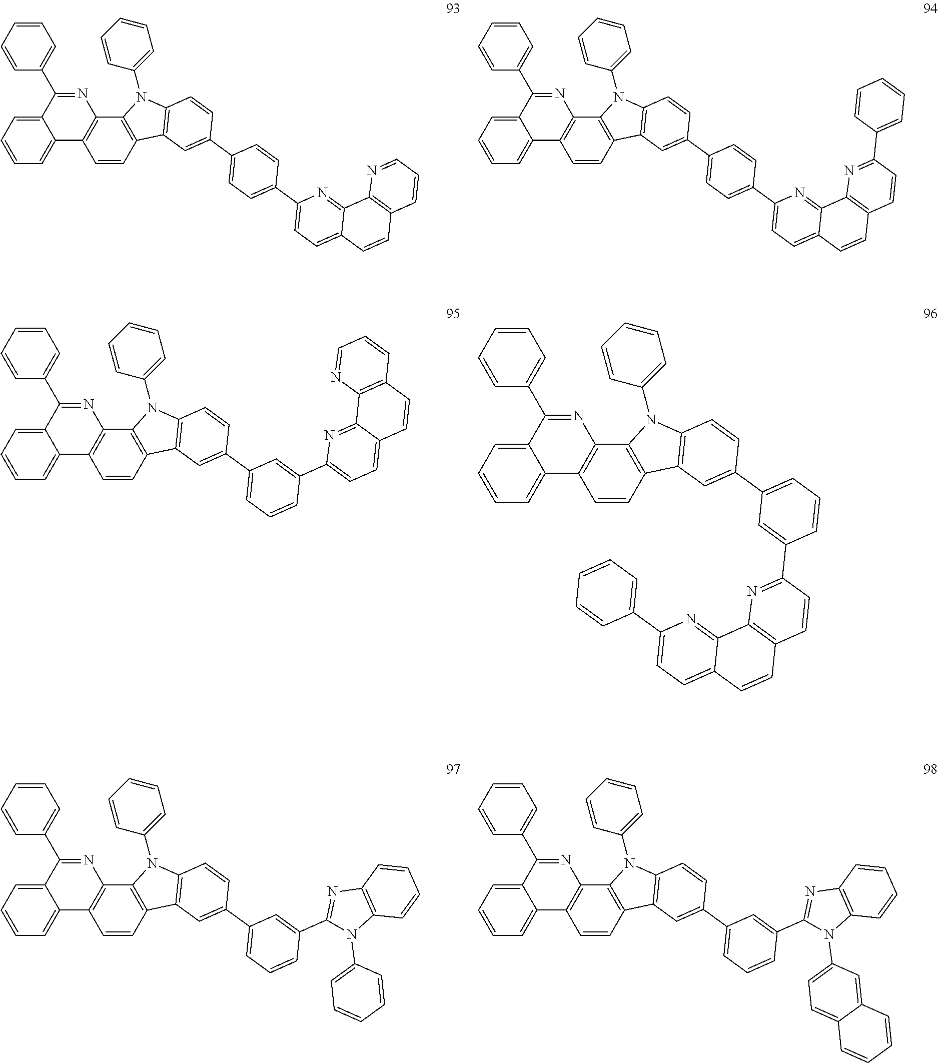

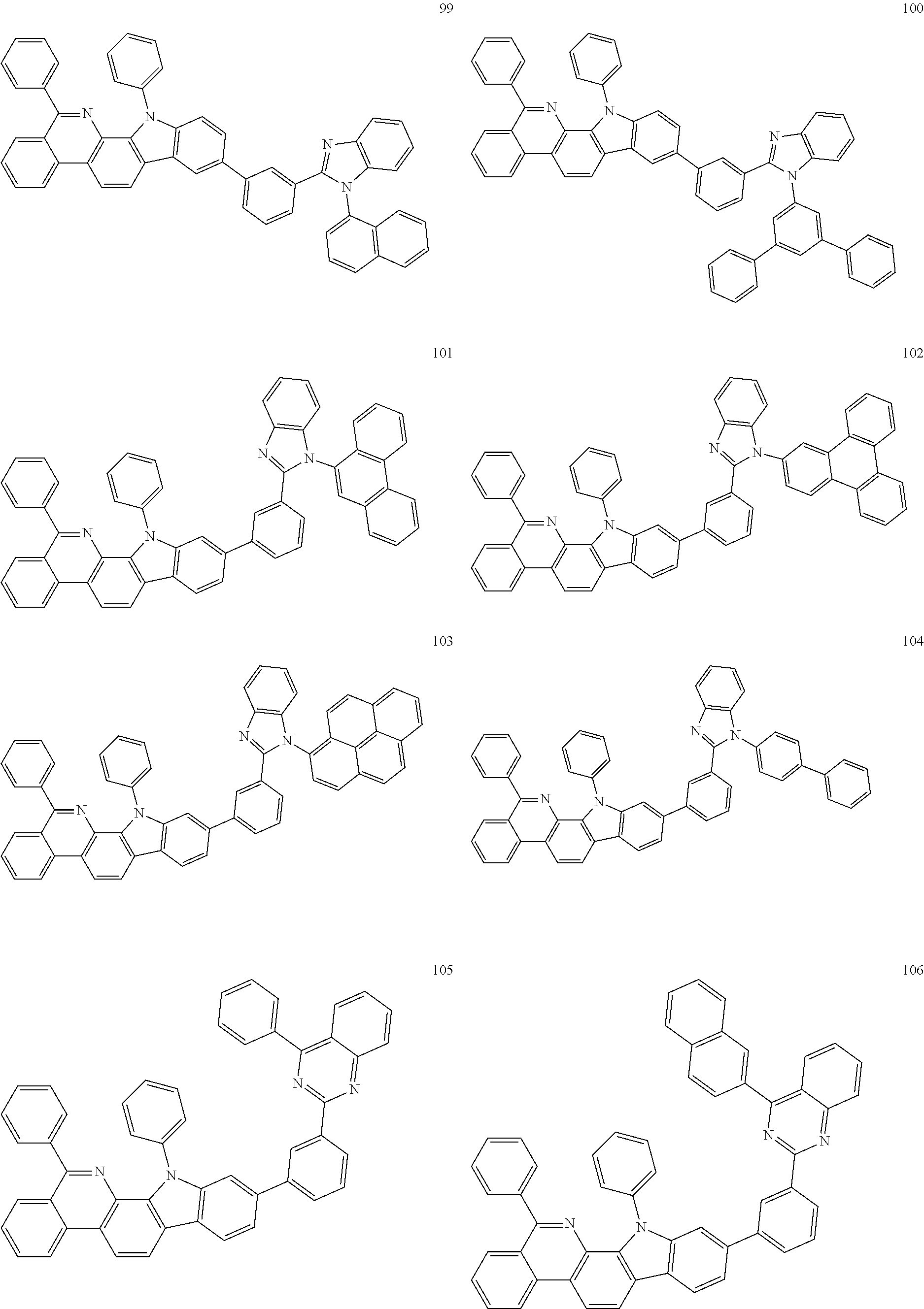

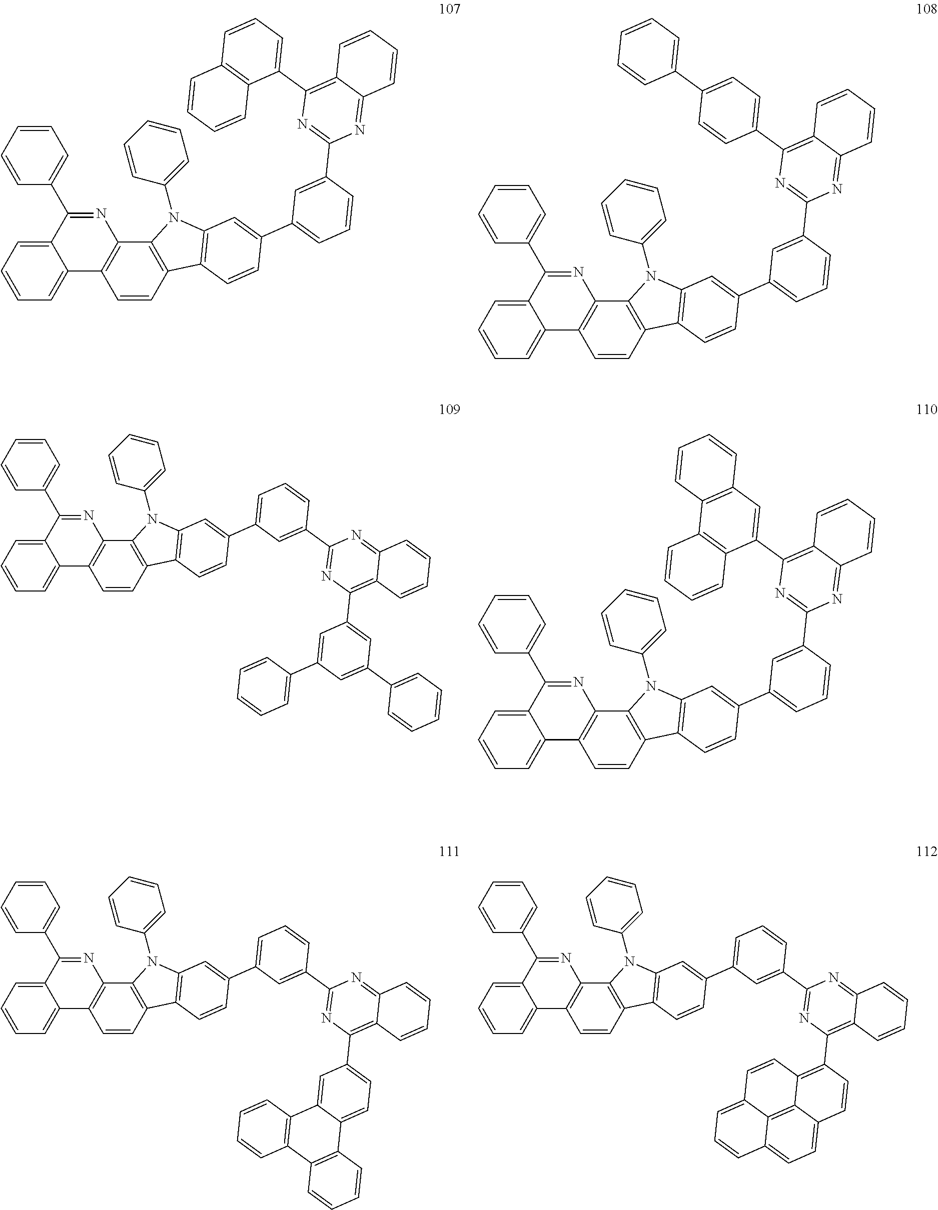

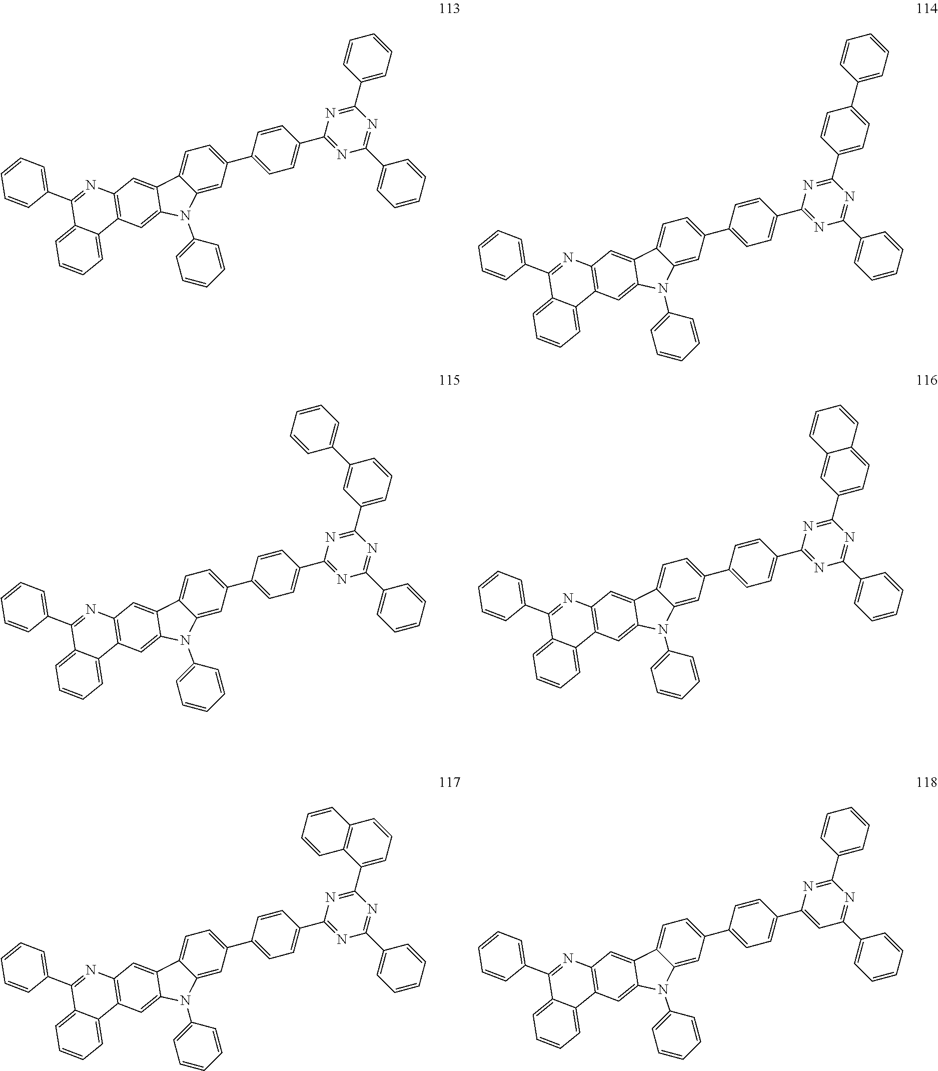

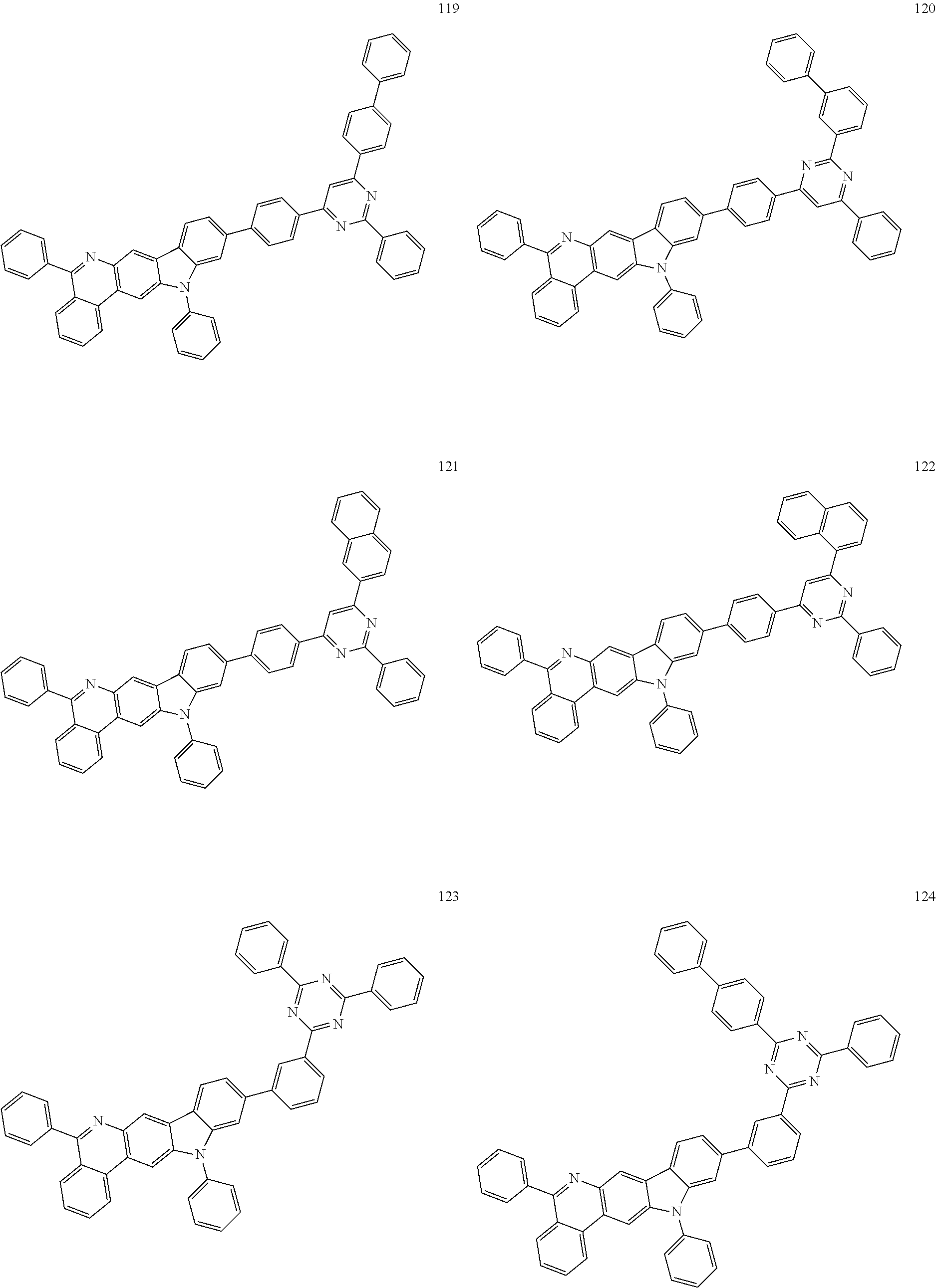

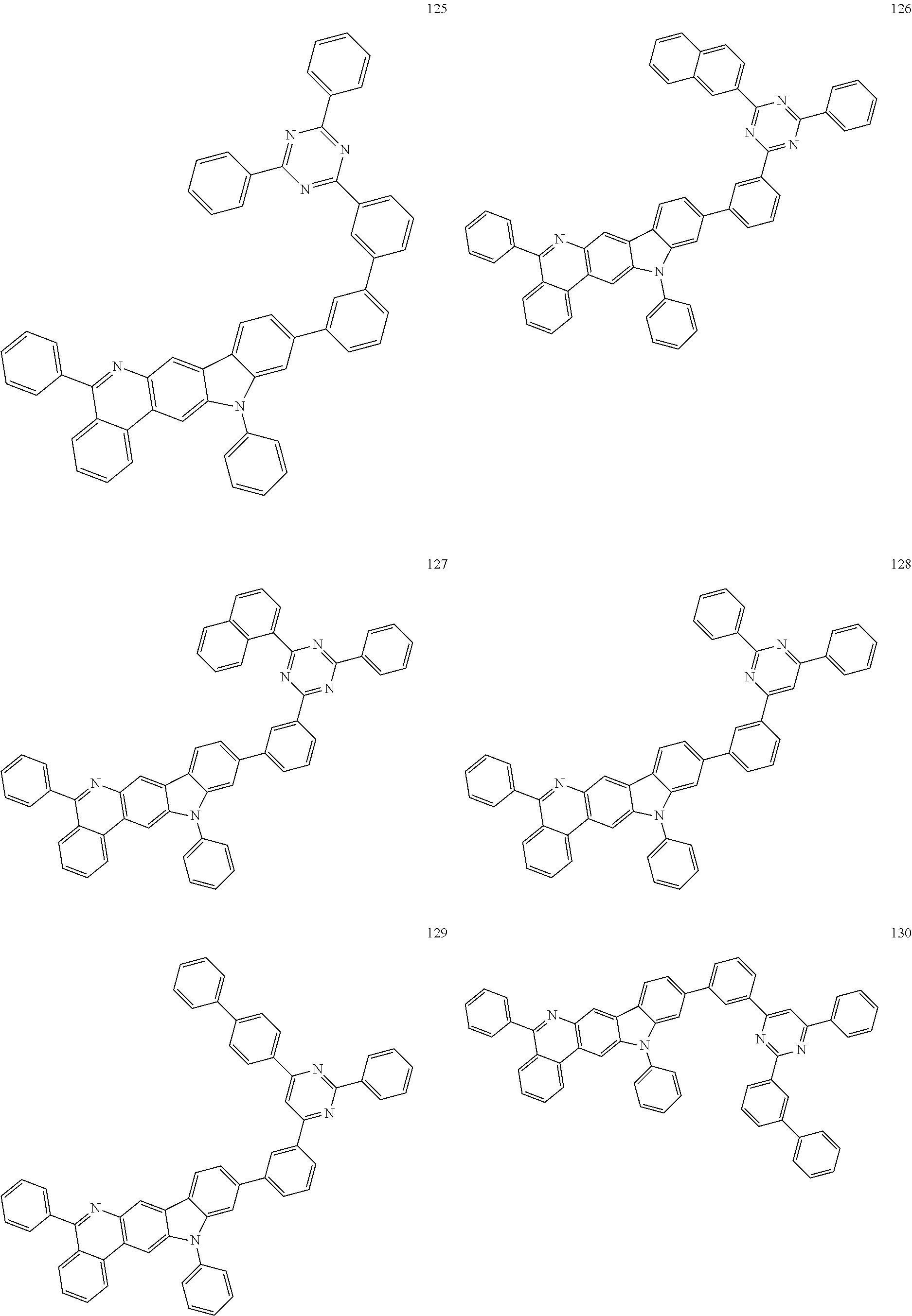

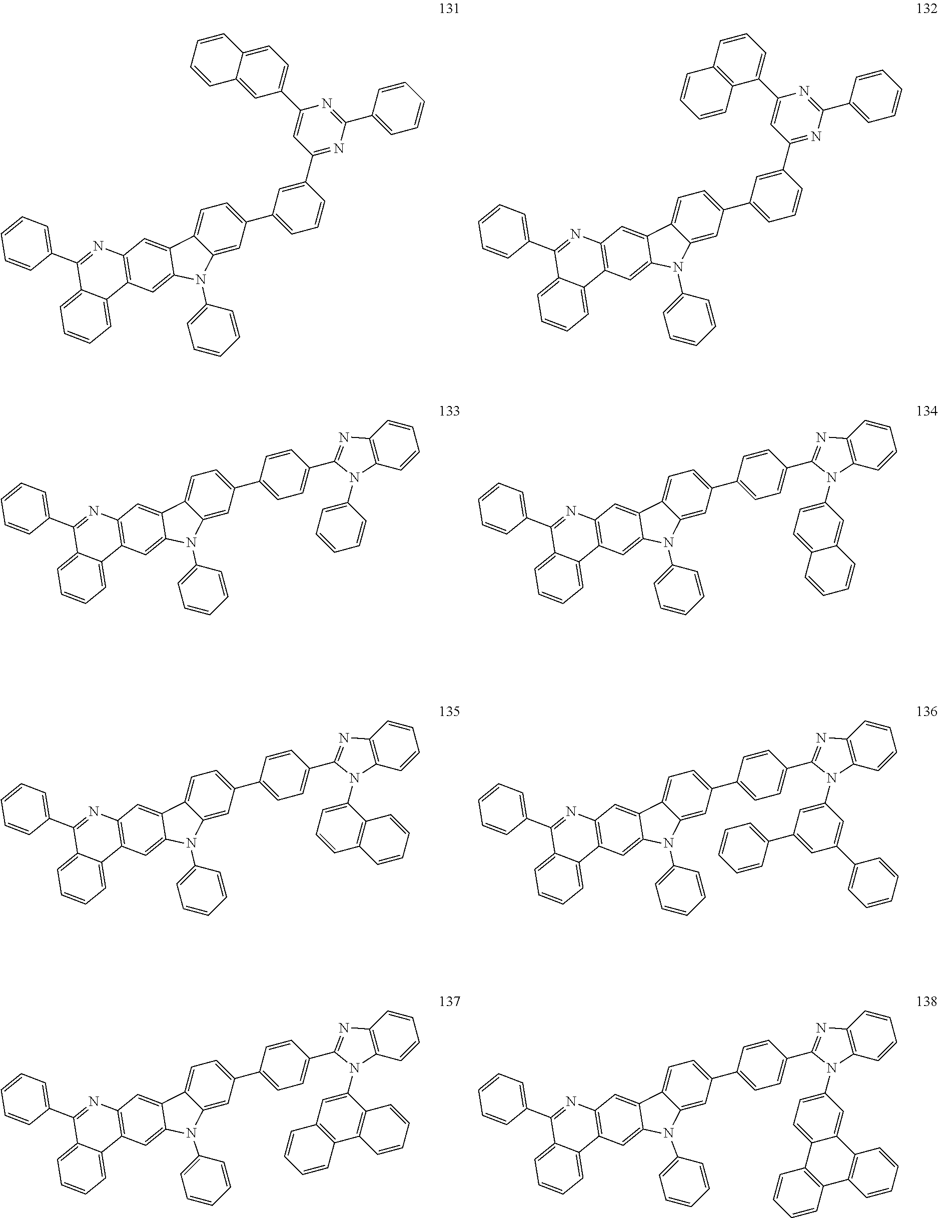

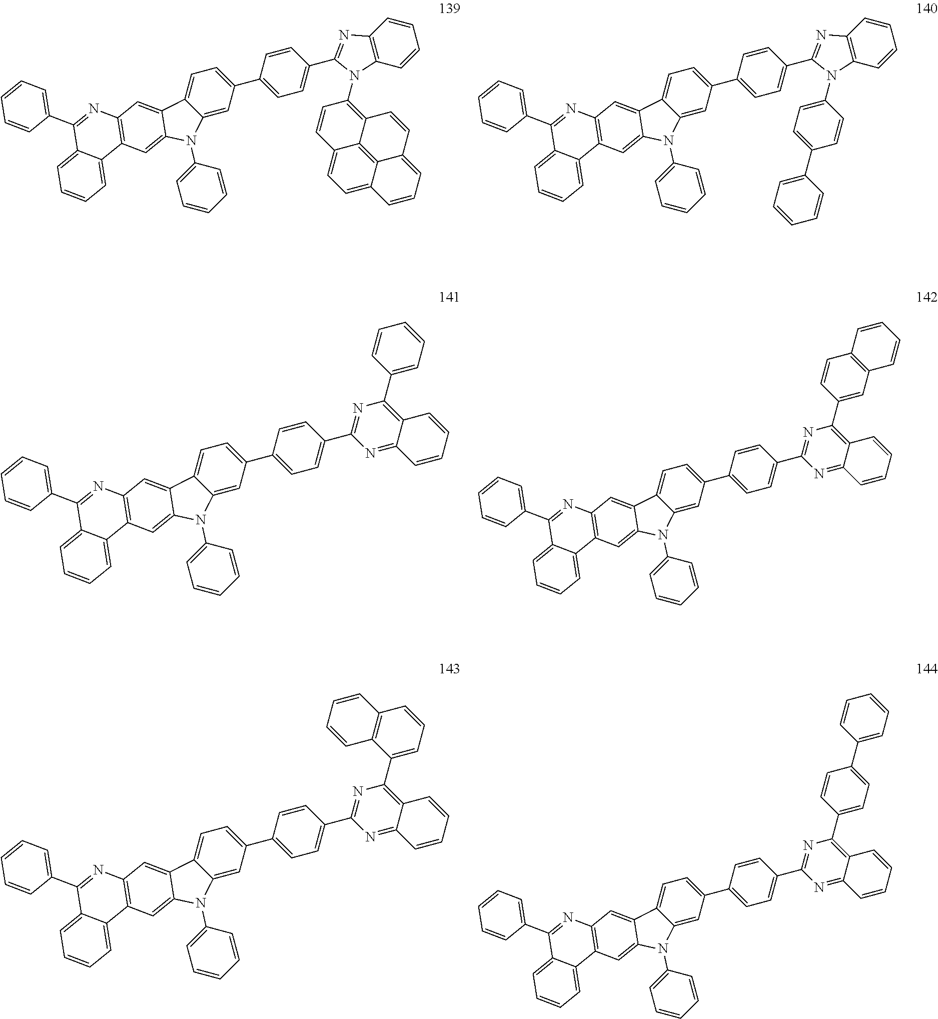

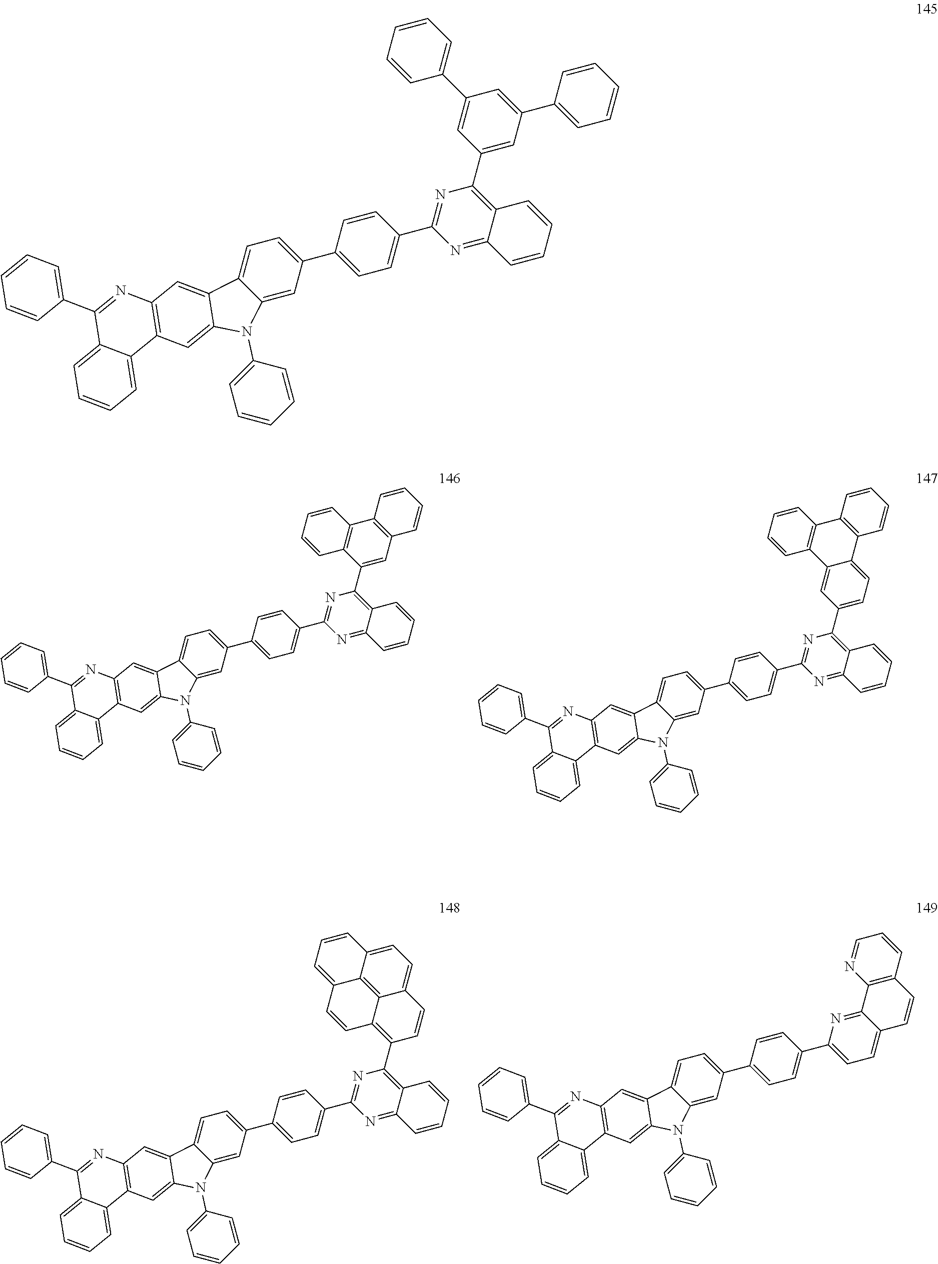

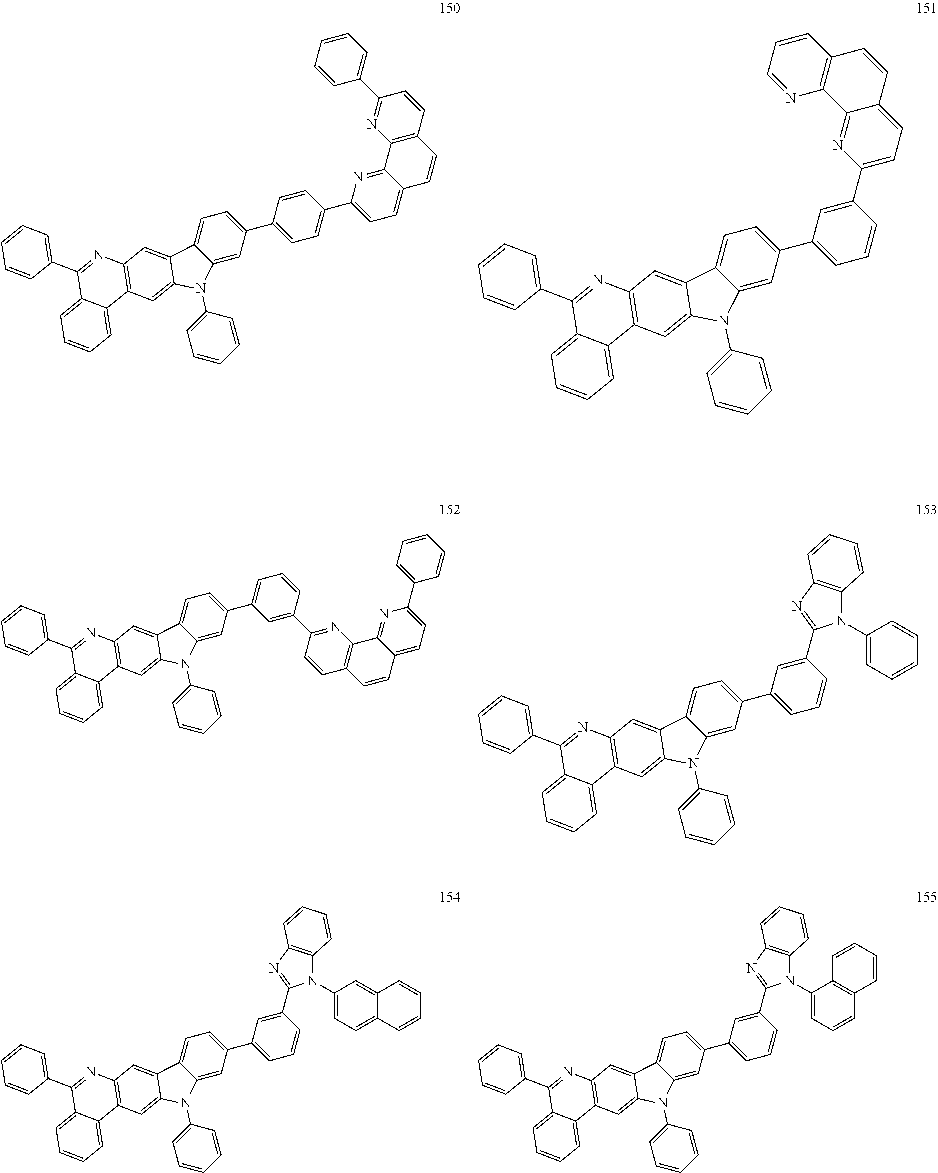

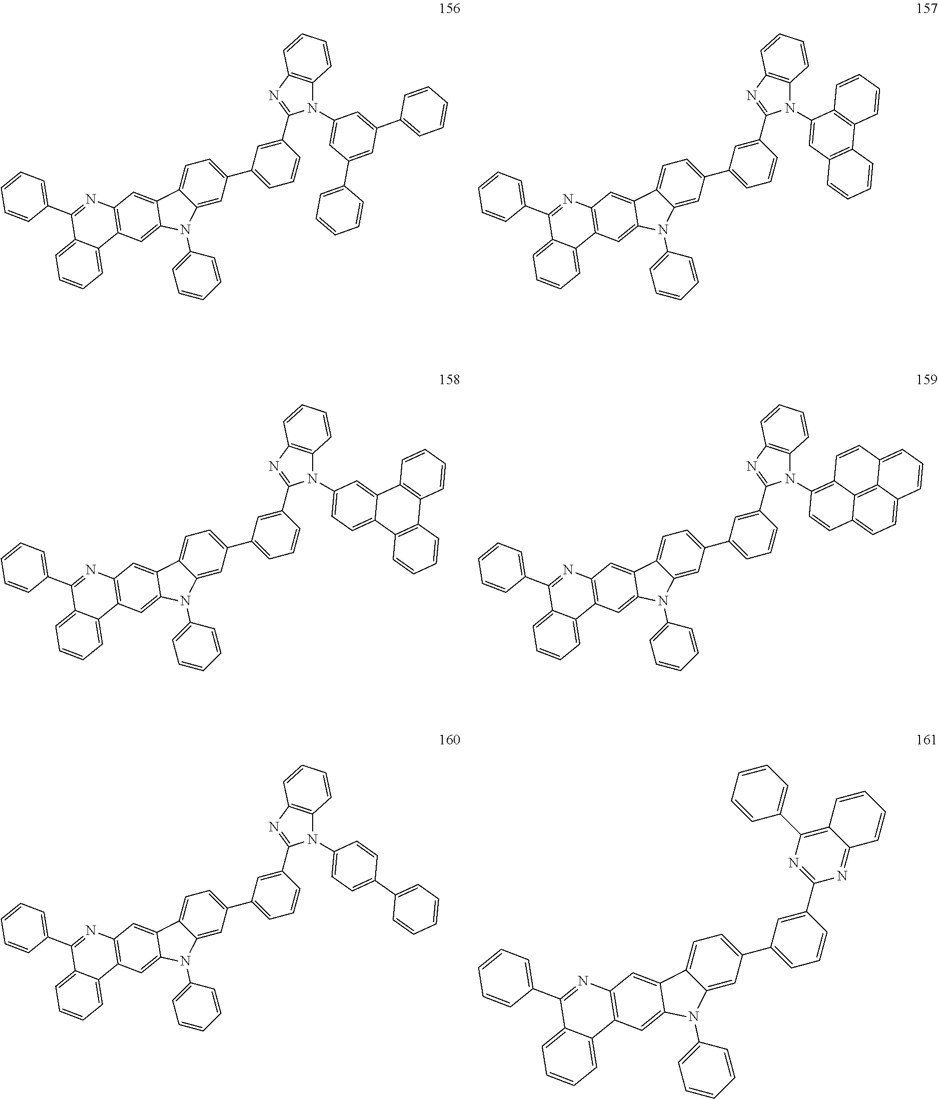

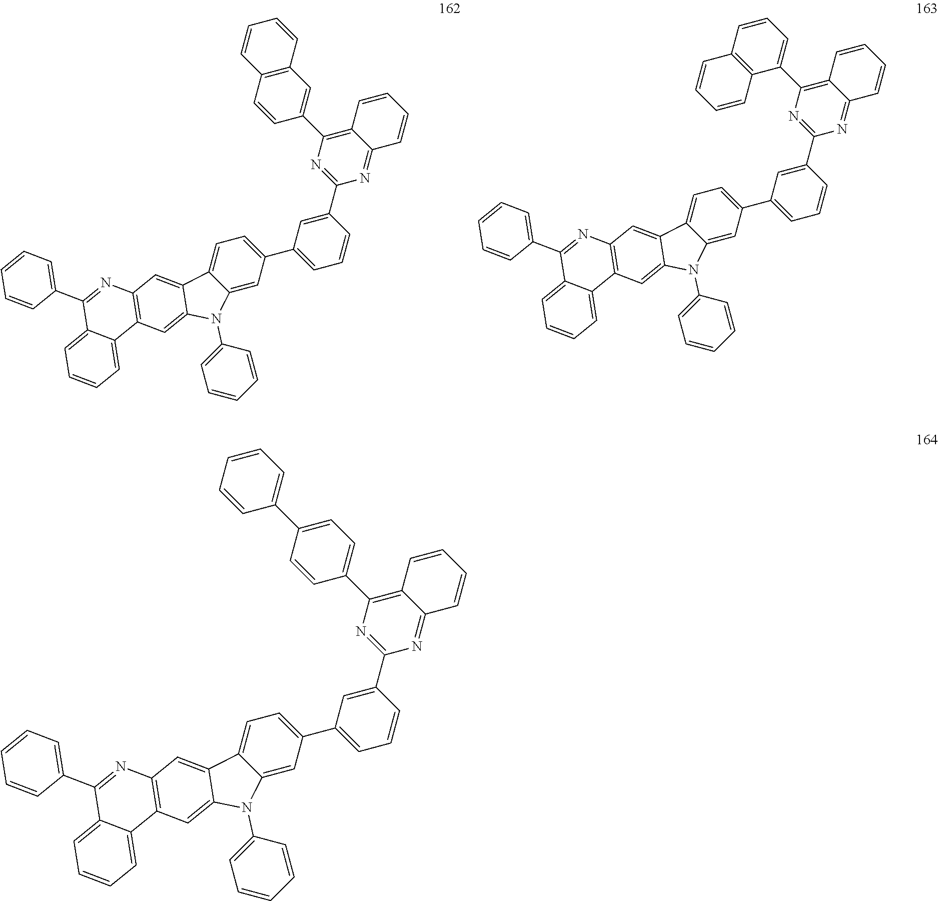 [SMILES: c1ccc(-c2nc3cc4c5ccc(-c6cccc(-c7nc(-c8ccc9ccccc9c8)c8ccccc8n7)c6)cc5n(-c5ccccc5)c4cc3c3ccccc23)cc1 ; c1ccc(-c2nc3cc4c5ccc(-c6cccc(-c7nc(-c8cccc9ccccc89)c8ccccc8n7)c6)cc5n(-c5ccccc5)c4cc3c3ccccc23)cc1 ; c1ccc(-c2ccc(-c3nc(-c4cccc(-c5ccc6c7cc8nc(-c9ccccc9)c9ccccc9c8cc7n(-c7ccccc7)c6c5)c4)nc4ccccc34)cc2)cc1]

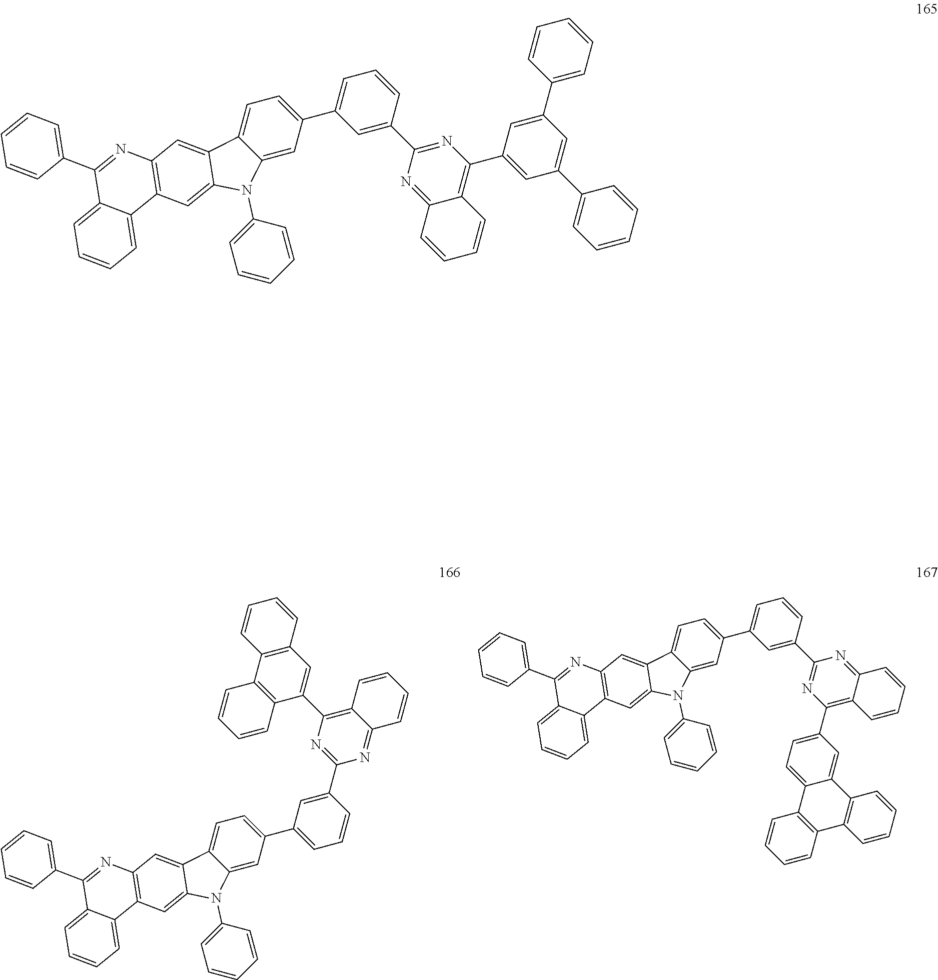 [SMILES: c1ccc(-c2cc(-c3ccccc3)cc(-c3nc(-c4cccc(-c5ccc6c7cc8nc(-c9ccccc9)c9ccccc9c8cc7n(-c7ccccc7)c6c5)c4)nc4ccccc34)c2)cc1 ; c1ccc(-c2nc3cc4c5ccc(-c6cccc(-c7nc(-c8cc9ccccc9c9ccccc89)c8ccccc8n7)c6)cc5n(-c5ccccc5)c4cc3c3ccccc23)cc1 ; c1ccc(-c2nc3cc4c5ccc(-c6cccc(-c7nc(-c8ccc9c%10ccccc%10c%10ccccc%10c9c8)c8ccccc8n7)c6)cc5n(-c5ccccc5)c4cc3c3ccccc23)cc1]

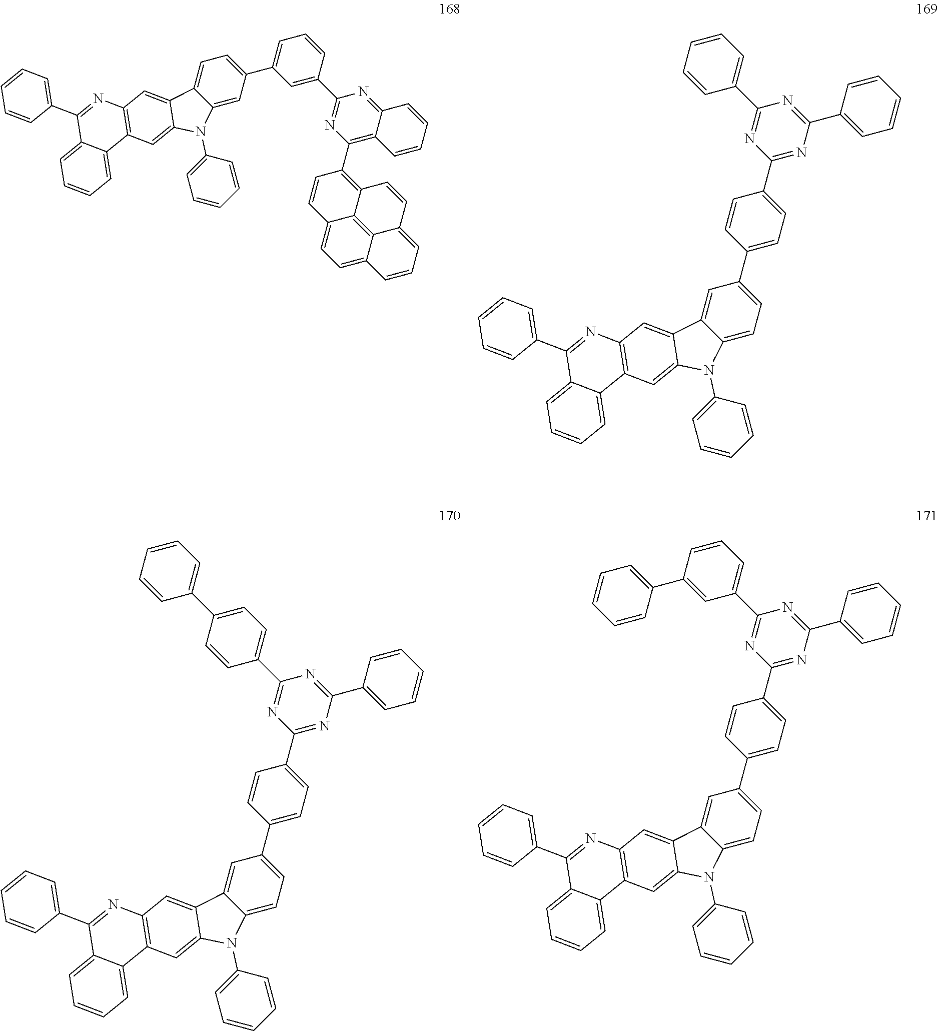 [SMILES: c1ccc(-c2nc3cc4c5ccc(-c6cccc(-c7nc(-c8ccc9ccc%10cccc%11ccc8c9c%10%11)c8ccccc8n7)c6)cc5n(-c5ccccc5)c4cc3c3ccccc23)cc1 ; c1ccc(-c2nc(-c3ccccc3)nc(-c3ccc(-c4ccc5c(c4)c4cc6nc(-c7ccccc7)c7ccccc7c6cc4n5-c4ccccc4)cc3)n2)cc1 ; c1ccc(-c2ccc(-c3nc(-c4ccccc4)nc(-c4ccc(-c5ccc6c(c5)c5cc7nc(-c8ccccc8)c8ccccc8c7cc5n6-c5ccccc5)cc4)n3)cc2)cc1 ; c1ccc(-c2cccc(-c3nc(-c4ccccc4)nc(-c4ccc(-c5ccc6c(c5)c5cc7nc(-c8ccccc8)c8ccccc8c7cc5n6-c5ccccc5)cc4)n3)c2)cc1]

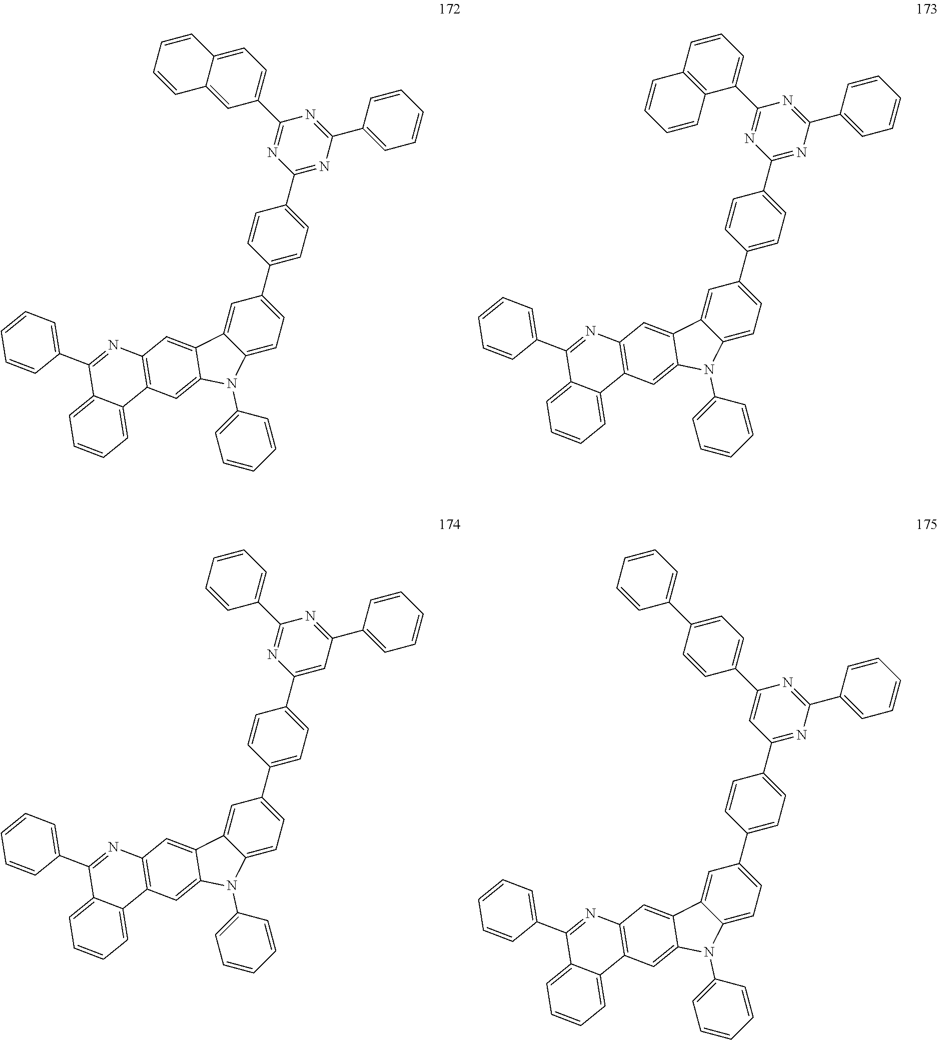 [SMILES: c1ccc(-c2nc(-c3ccc(-c4ccc5c(c4)c4cc6nc(-c7ccccc7)c7ccccc7c6cc4n5-c4ccccc4)cc3)nc(-c3ccc4ccccc4c3)n2)cc1 ; c1ccc(-c2nc(-c3ccc(-c4ccc5c(c4)c4cc6nc(-c7ccccc7)c7ccccc7c6cc4n5-c4ccccc4)cc3)nc(-c3cccc4ccccc34)n2)cc1 ; c1ccc(-c2cc(-c3ccc(-c4ccc5c(c4)c4cc6nc(-c7ccccc7)c7ccccc7c6cc4n5-c4ccccc4)cc3)nc(-c3ccccc3)n2)cc1 ; c1ccc(-c2ccc(-c3cc(-c4ccc(-c5ccc6c(c5)c5cc7nc(-c8ccccc8)c8ccccc8c7cc5n6-c5ccccc5)cc4)nc(-c4ccccc4)n3)cc2)cc1]

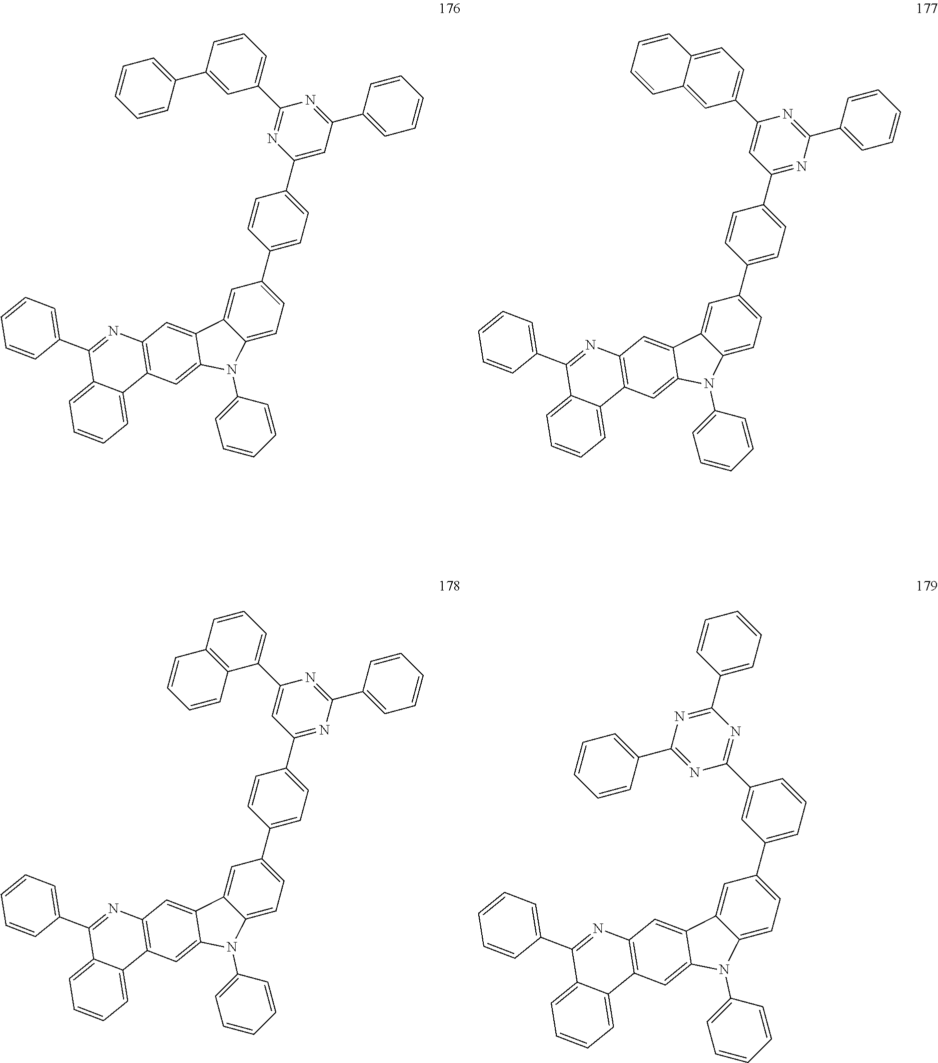 [SMILES: c1ccc(-c2cccc(-c3nc(-c4ccccc4)cc(-c4ccc(-c5ccc6c(c5)c5cc7nc(-c8ccccc8)c8ccccc8c7cc5n6-c5ccccc5)cc4)n3)c2)cc1 ; c1ccc(-c2nc(-c3ccc(-c4ccc5c(c4)c4cc6nc(-c7ccccc7)c7ccccc7c6cc4n5-c4ccccc4)cc3)cc(-c3ccc4ccccc4c3)n2)cc1 ; c1ccc(-c2nc(-c3ccc(-c4ccc5c(c4)c4cc6nc(-c7ccccc7)c7ccccc7c6cc4n5-c4ccccc4)cc3)cc(-c3cccc4ccccc34)n2)cc1 ; c1ccc(-c2nc(-c3ccccc3)nc(-c3cccc(-c4ccc5c(c4)c4cc6nc(-c7ccccc7)c7ccccc7c6cc4n5-c4ccccc4)c3)n2)cc1]

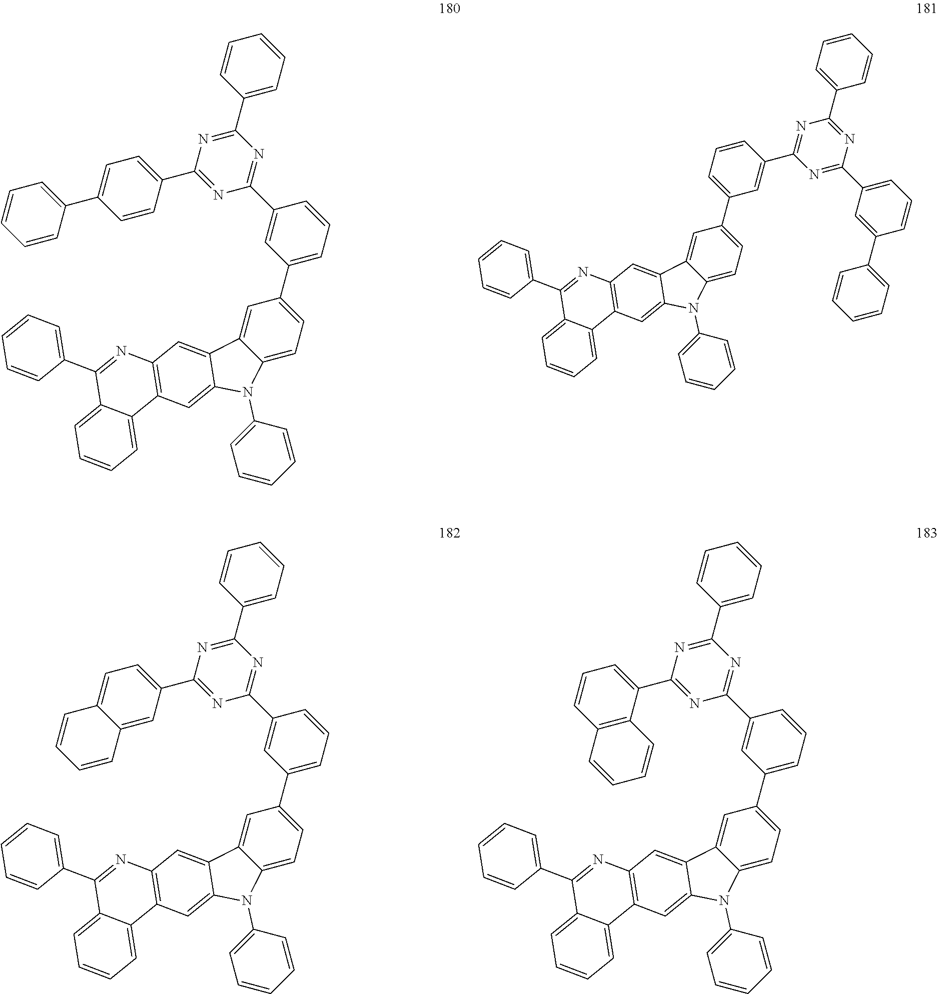 [SMILES: c1ccc(-c2ccc(-c3nc(-c4ccccc4)nc(-c4cccc(-c5ccc6c(c5)c5cc7nc(-c8ccccc8)c8ccccc8c7cc5n6-c5ccccc5)c4)n3)cc2)cc1 ; c1ccc(-c2cccc(-c3nc(-c4ccccc4)nc(-c4cccc(-c5ccc6c(c5)c5cc7nc(-c8ccccc8)c8ccccc8c7cc5n6-c5ccccc5)c4)n3)c2)cc1 ; c1ccc(-c2nc(-c3cccc(-c4ccc5c(c4)c4cc6nc(-c7ccccc7)c7ccccc7c6cc4n5-c4ccccc4)c3)nc(-c3ccc4ccccc4c3)n2)cc1 ; c1ccc(-c2nc(-c3cccc(-c4ccc5c(c4)c4cc6nc(-c7ccccc7)c7ccccc7c6cc4n5-c4ccccc4)c3)nc(-c3cccc4ccccc34)n2)cc1]

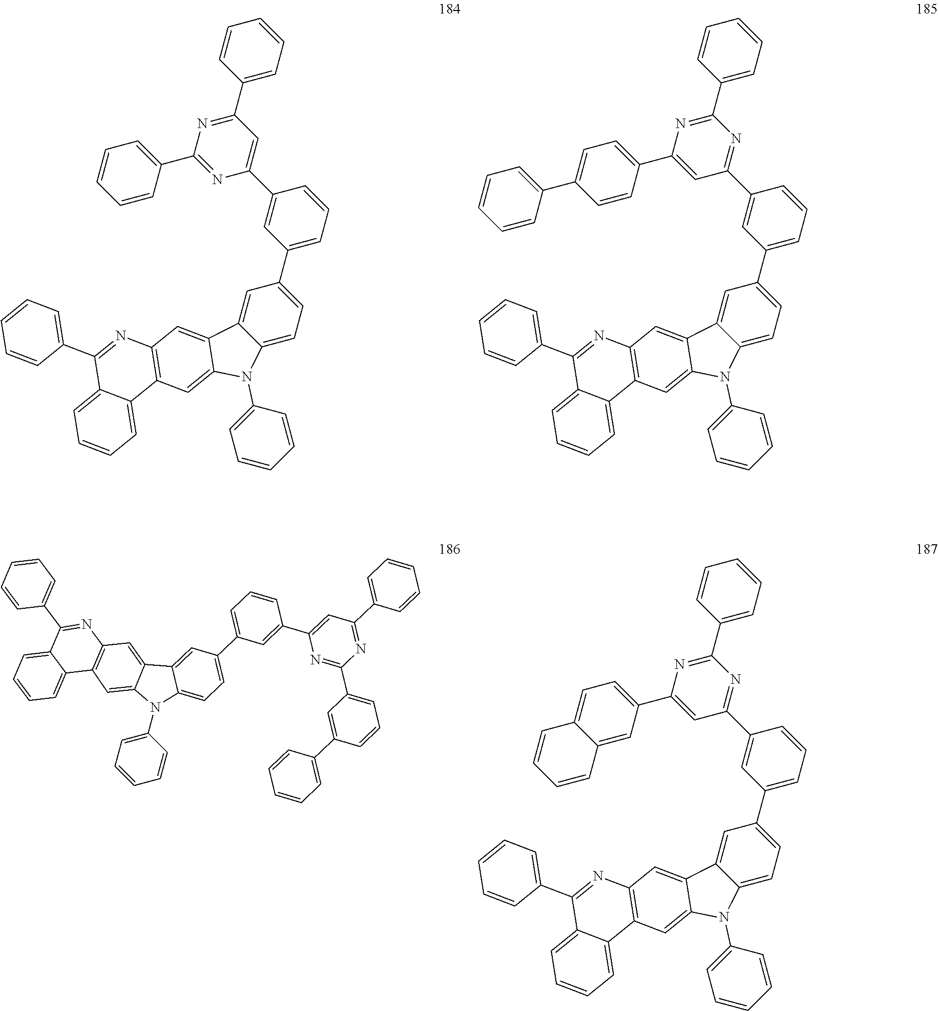 [SMILES: c1ccc(-c2cc(-c3cccc(-c4ccc5c(c4)c4cc6nc(-c7ccccc7)c7ccccc7c6cc4n5-c4ccccc4)c3)nc(-c3ccccc3)n2)cc1 ; c1ccc(-c2ccc(-c3cc(-c4cccc(-c5ccc6c(c5)c5cc7nc(-c8ccccc8)c8ccccc8c7cc5n6-c5ccccc5)c4)nc(-c4ccccc4)n3)cc2)cc1 ; c1ccc(-c2cccc(-c3nc(-c4ccccc4)cc(-c4cccc(-c5ccc6c(c5)c5cc7nc(-c8ccccc8)c8ccccc8c7cc5n6-c5ccccc5)c4)n3)c2)cc1 ; c1ccc(-c2nc(-c3cccc(-c4ccc5c(c4)c4cc6nc(-c7ccccc7)c7ccccc7c6cc4n5-c4ccccc4)c3)cc(-c3ccc4ccccc4c3)n2)cc1]

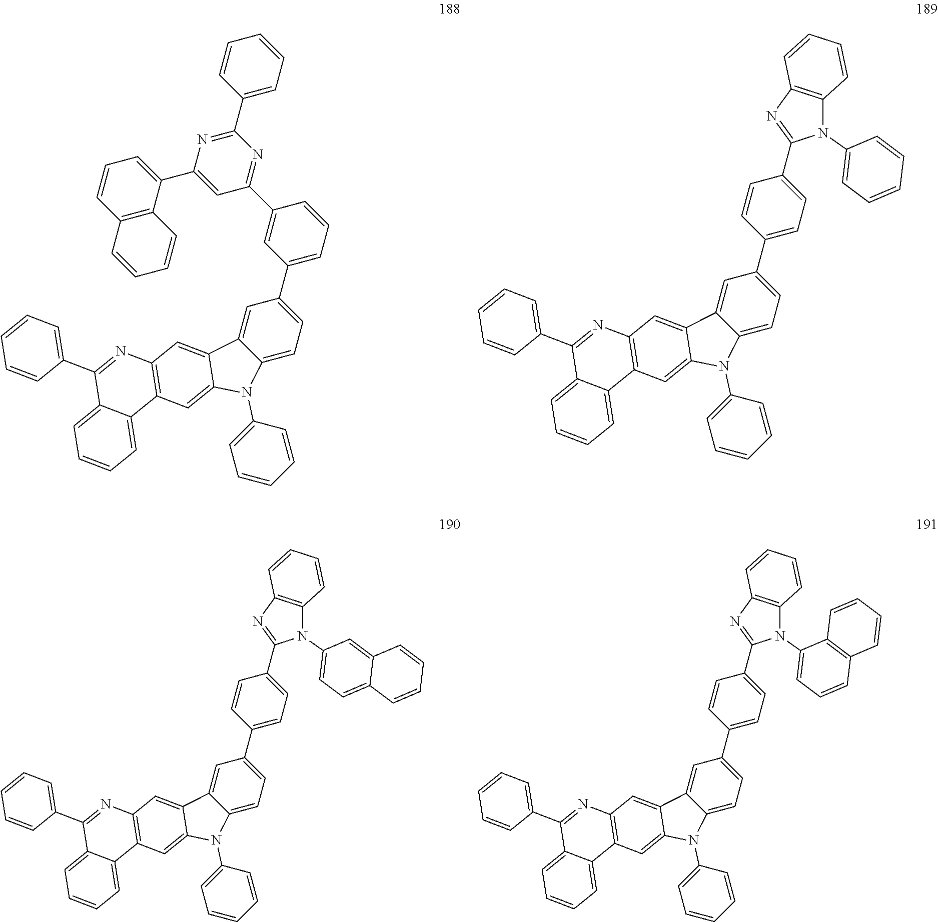 [SMILES: c1ccc(-c2nc(-c3cccc(-c4ccc5c(c4)c4cc6nc(-c7ccccc7)c7ccccc7c6cc4n5-c4ccccc4)c3)cc(-c3cccc4ccccc34)n2)cc1 ; c1ccc(-c2nc3cc4c5cc(-c6ccc(-c7nc8ccccc8n7-c7ccccc7)cc6)ccc5n(-c5ccccc5)c4cc3c3ccccc23)cc1 ; c1ccc(-c2nc3cc4c5cc(-c6ccc(-c7nc8ccccc8n7-c7ccc8ccccc8c7)cc6)ccc5n(-c5ccccc5)c4cc3c3ccccc23)cc1 ; c1ccc(-c2nc3cc4c5cc(-c6ccc(-c7nc8ccccc8n7-c7cccc8ccccc78)cc6)ccc5n(-c5ccccc5)c4cc3c3ccccc23)cc1]

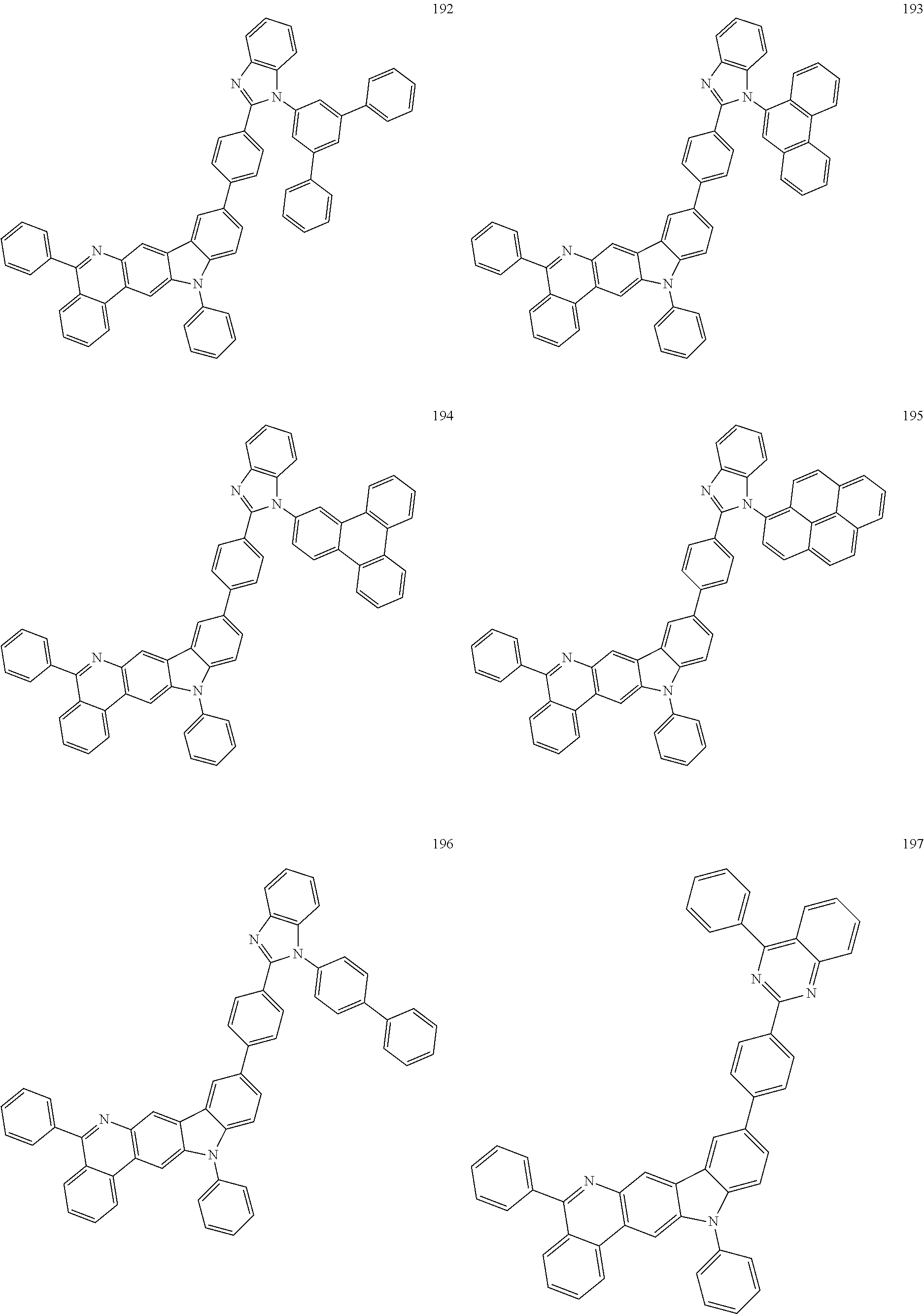

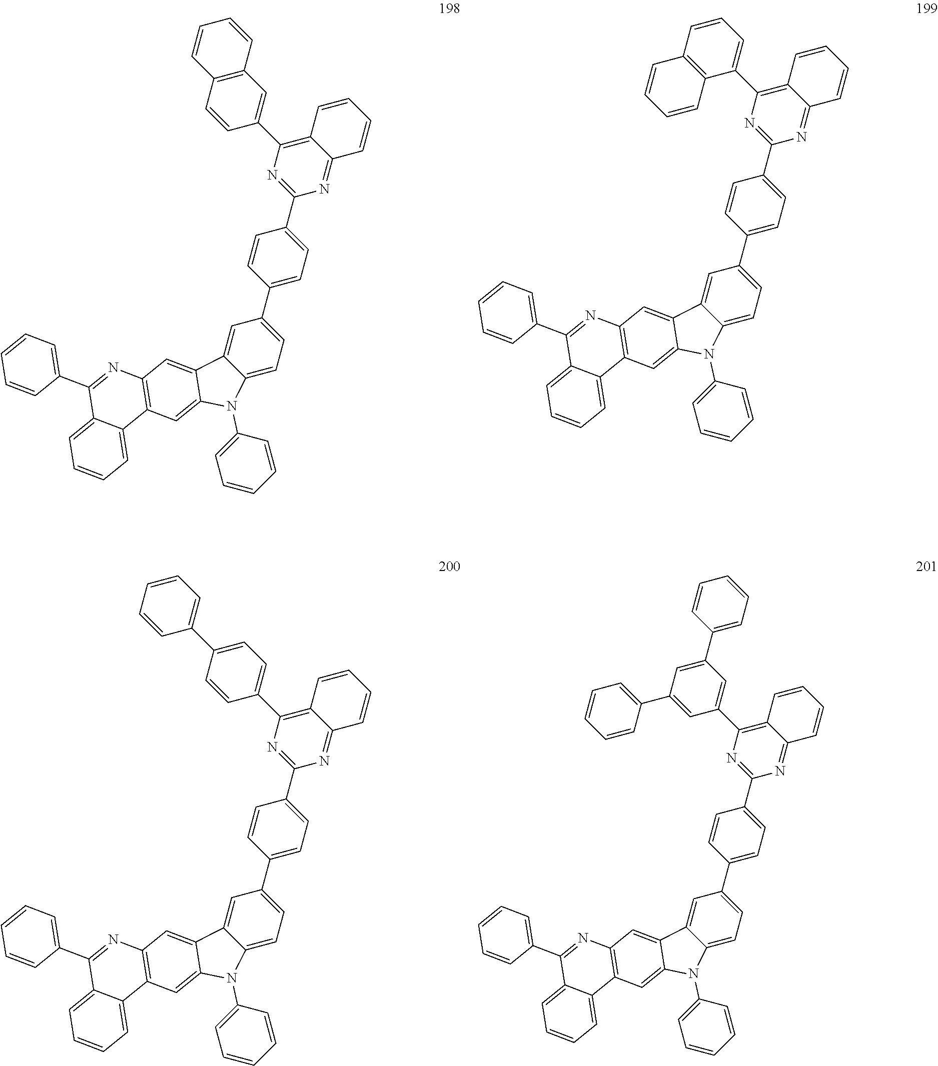 [SMILES: c1ccc(-c2nc3cc4c5cc(-c6ccc(-c7nc(-c8ccc9ccccc9c8)c8ccccc8n7)cc6)ccc5n(-c5ccccc5)c4cc3c3ccccc23)cc1 ; c1ccc(-c2nc3cc4c5cc(-c6ccc(-c7nc(-c8cccc9ccccc89)c8ccccc8n7)cc6)ccc5n(-c5ccccc5)c4cc3c3ccccc23)cc1 ; c1ccc(-c2ccc(-c3nc(-c4ccc(-c5ccc6c(c5)c5cc7nc(-c8ccccc8)c8ccccc8c7cc5n6-c5ccccc5)cc4)nc4ccccc34)cc2)cc1 ; c1ccc(-c2cc(-c3ccccc3)cc(-c3nc(-c4ccc(-c5ccc6c(c5)c5cc7nc(-c8ccccc8)c8ccccc8c7cc5n6-c5ccccc5)cc4)nc4ccccc34)c2)cc1]

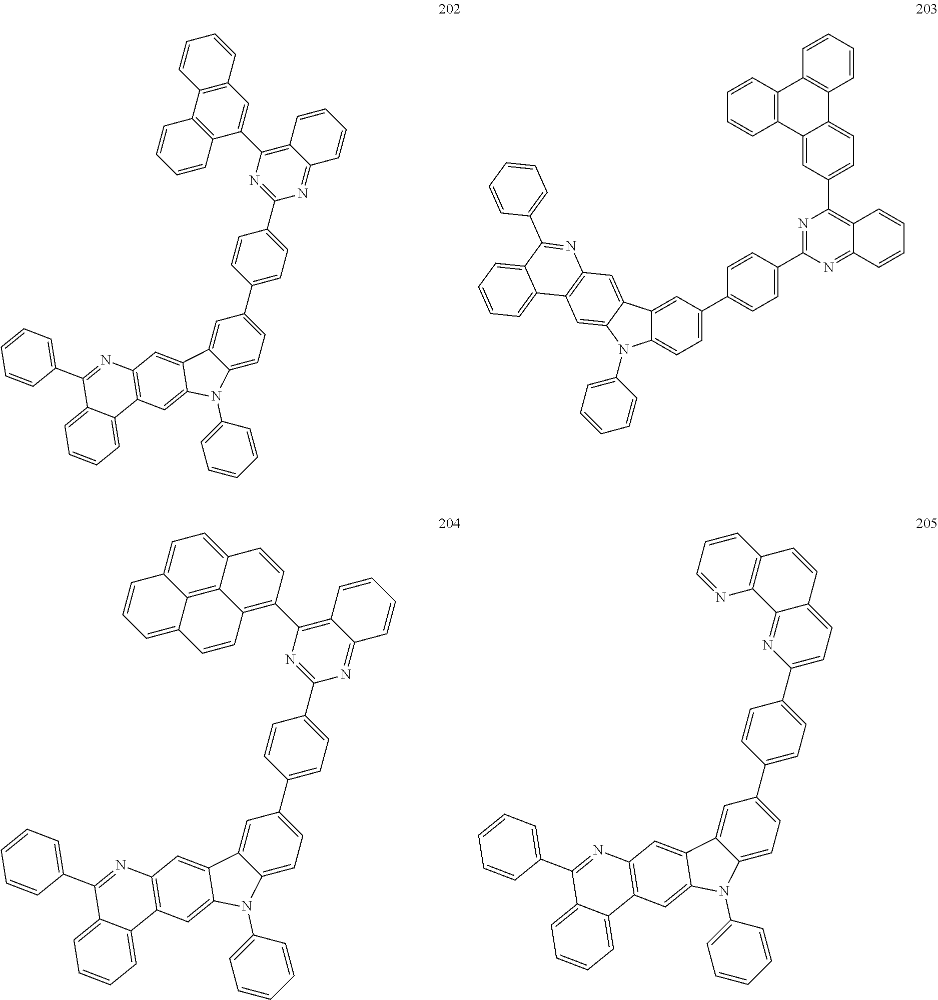 [SMILES: c1ccc(-c2nc3cc4c5cc(-c6ccc(-c7nc(-c8cc9ccccc9c9ccccc89)c8ccccc8n7)cc6)ccc5n(-c5ccccc5)c4cc3c3ccccc23)cc1 ; c1ccc(-c2nc3cc4c5cc(-c6ccc(-c7nc(-c8ccc9c%10ccccc%10c%10ccccc%10c9c8)c8ccccc8n7)cc6)ccc5n(-c5ccccc5)c4cc3c3ccccc23)cc1 ; c1ccc(-c2nc3cc4c5cc(-c6ccc(-c7nc(-c8ccc9ccc%10cccc%11ccc8c9c%10%11)c8ccccc8n7)cc6)ccc5n(-c5ccccc5)c4cc3c3ccccc23)cc1 ; c1ccc(-c2nc3cc4c5cc(-c6ccc(-c7ccc8ccc9cccnc9c8n7)cc6)ccc5n(-c5ccccc5)c4cc3c3ccccc23)cc1]

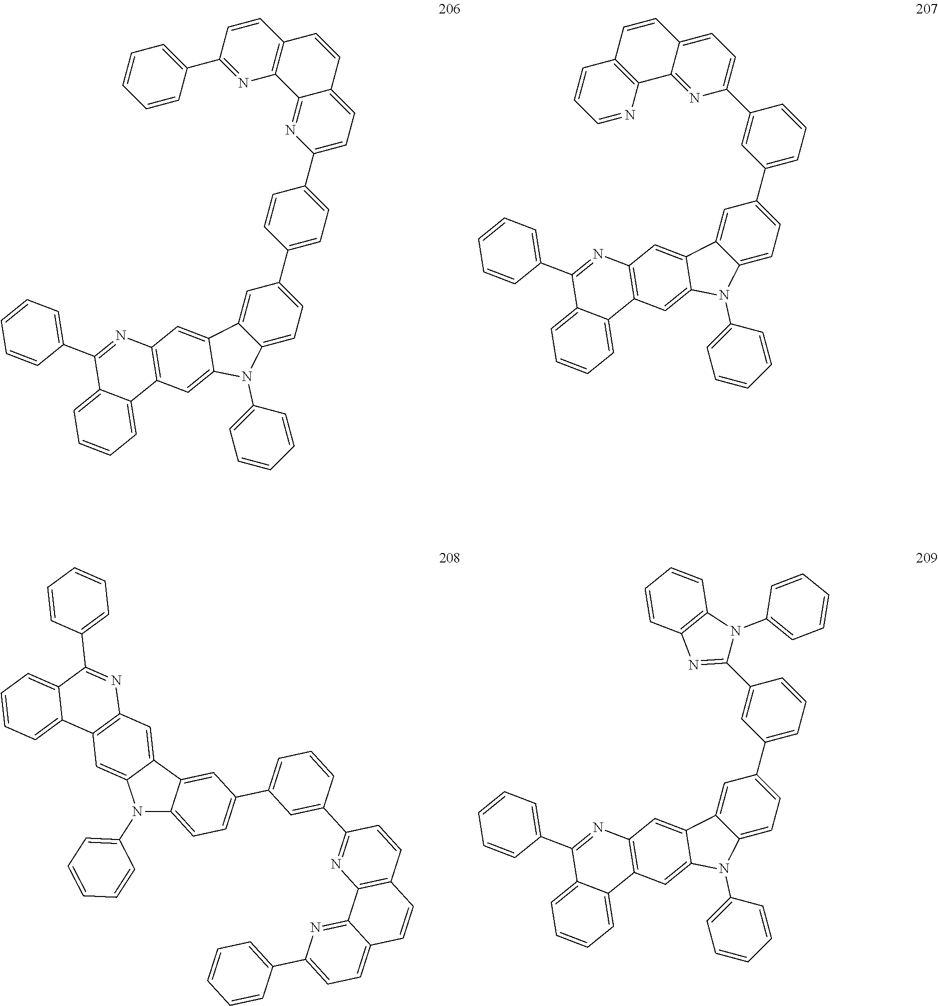 [SMILES: c1ccc(-c2ccc3ccc4ccc(-c5ccc(-c6ccc7c(c6)c6cc8nc(-c9ccccc9)c9ccccc9c8cc6n7-c6ccccc6)cc5)nc4c3n2)cc1 ; c1ccc(-c2nc3cc4c5cc(-c6cccc(-c7ccc8ccc9cccnc9c8n7)c6)ccc5n(-c5ccccc5)c4cc3c3ccccc23)cc1 ; c1ccc(-c2ccc3ccc4ccc(-c5cccc(-c6ccc7c(c6)c6cc8nc(-c9ccccc9)c9ccccc9c8cc6n7-c6ccccc6)c5)nc4c3n2)cc1 ; c1ccc(-c2nc3cc4c5cc(-c6cccc(-c7nc8ccccc8n7-c7ccccc7)c6)ccc5n(-c5ccccc5)c4cc3c3ccccc23)cc1]

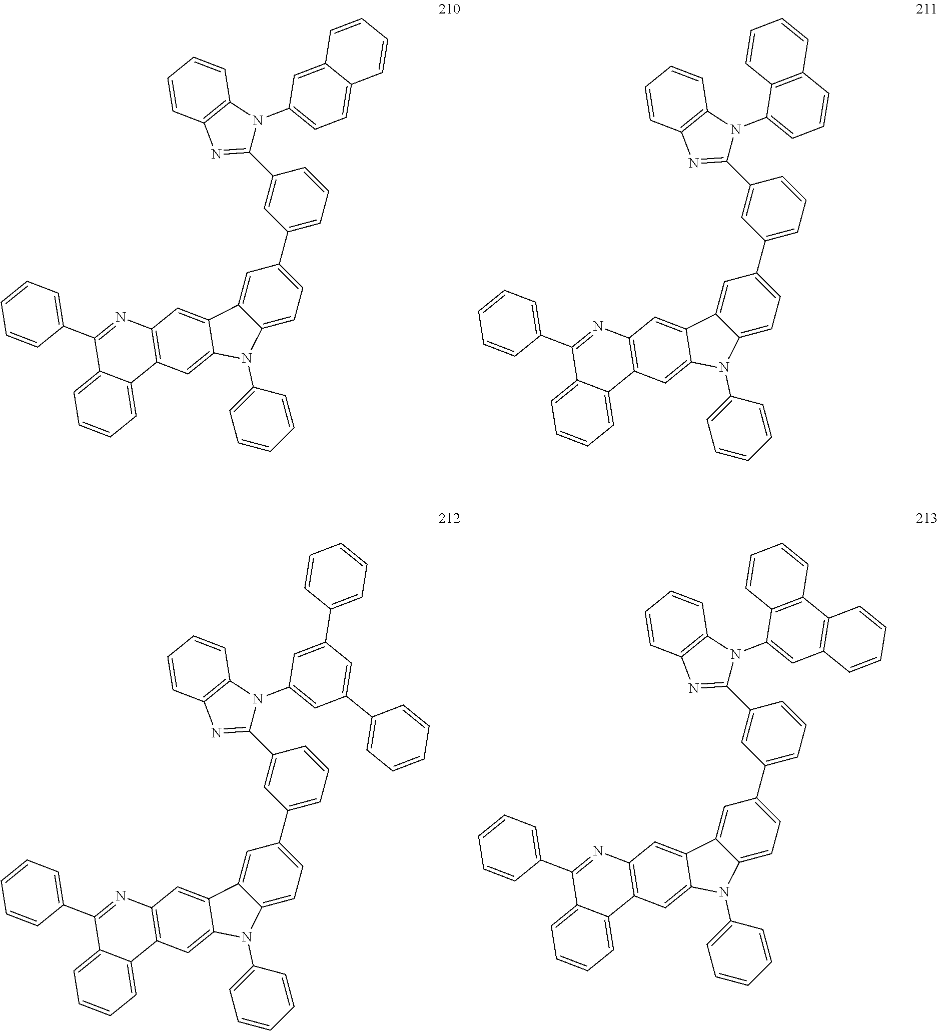 [SMILES: c1ccc(-c2nc3cc4c5cc(-c6cccc(-c7nc8ccccc8n7-c7ccc8ccccc8c7)c6)ccc5n(-c5ccccc5)c4cc3c3ccccc23)cc1 ; c1ccc(-c2nc3cc4c5cc(-c6cccc(-c7nc8ccccc8n7-c7cccc8ccccc78)c6)ccc5n(-c5ccccc5)c4cc3c3ccccc23)cc1 ; c1ccc(-c2cc(-c3ccccc3)cc(-n3c(-c4cccc(-c5ccc6c(c5)c5cc7nc(-c8ccccc8)c8ccccc8c7cc5n6-c5ccccc5)c4)nc4ccccc43)c2)cc1 ; c1ccc(-c2nc3cc4c5cc(-c6cccc(-c7nc8ccccc8n7-c7cc8ccccc8c8ccccc78)c6)ccc5n(-c5ccccc5)c4cc3c3ccccc23)cc1]

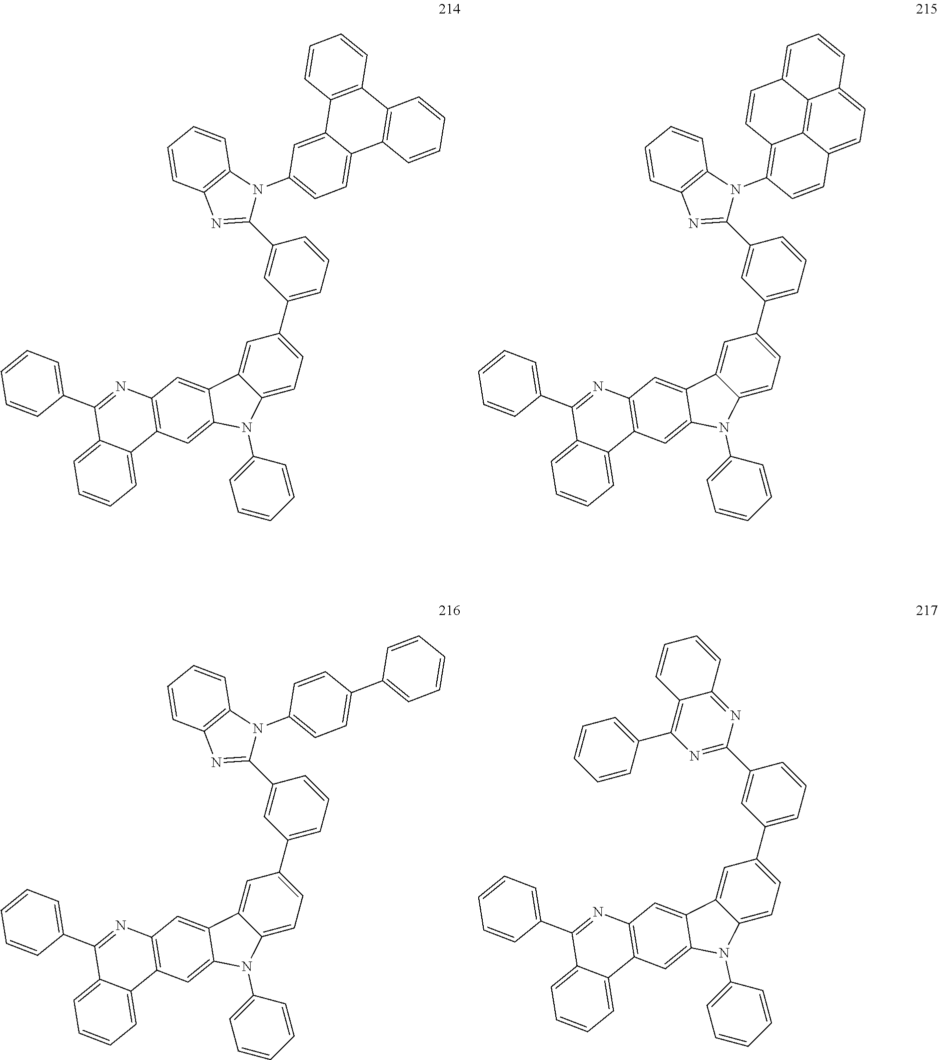 [SMILES: c1ccc(-c2nc3cc4c5cc(-c6cccc(-c7nc8ccccc8n7-c7ccc8c9ccccc9c9ccccc9c8c7)c6)ccc5n(-c5ccccc5)c4cc3c3ccccc23)cc1 ; c1ccc(-c2nc3cc4c5cc(-c6cccc(-c7nc8ccccc8n7-c7ccc8ccc9cccc%10ccc7c8c9%10)c6)ccc5n(-c5ccccc5)c4cc3c3ccccc23)cc1 ; c1ccc(-c2ccc(-n3c(-c4cccc(-c5ccc6c(c5)c5cc7nc(-c8ccccc8)c8ccccc8c7cc5n6-c5ccccc5)c4)nc4ccccc43)cc2)cc1 ; c1ccc(-c2nc(-c3cccc(-c4ccc5c(c4)c4cc6nc(-c7ccccc7)c7ccccc7c6cc4n5-c4ccccc4)c3)nc3ccccc23)cc1]

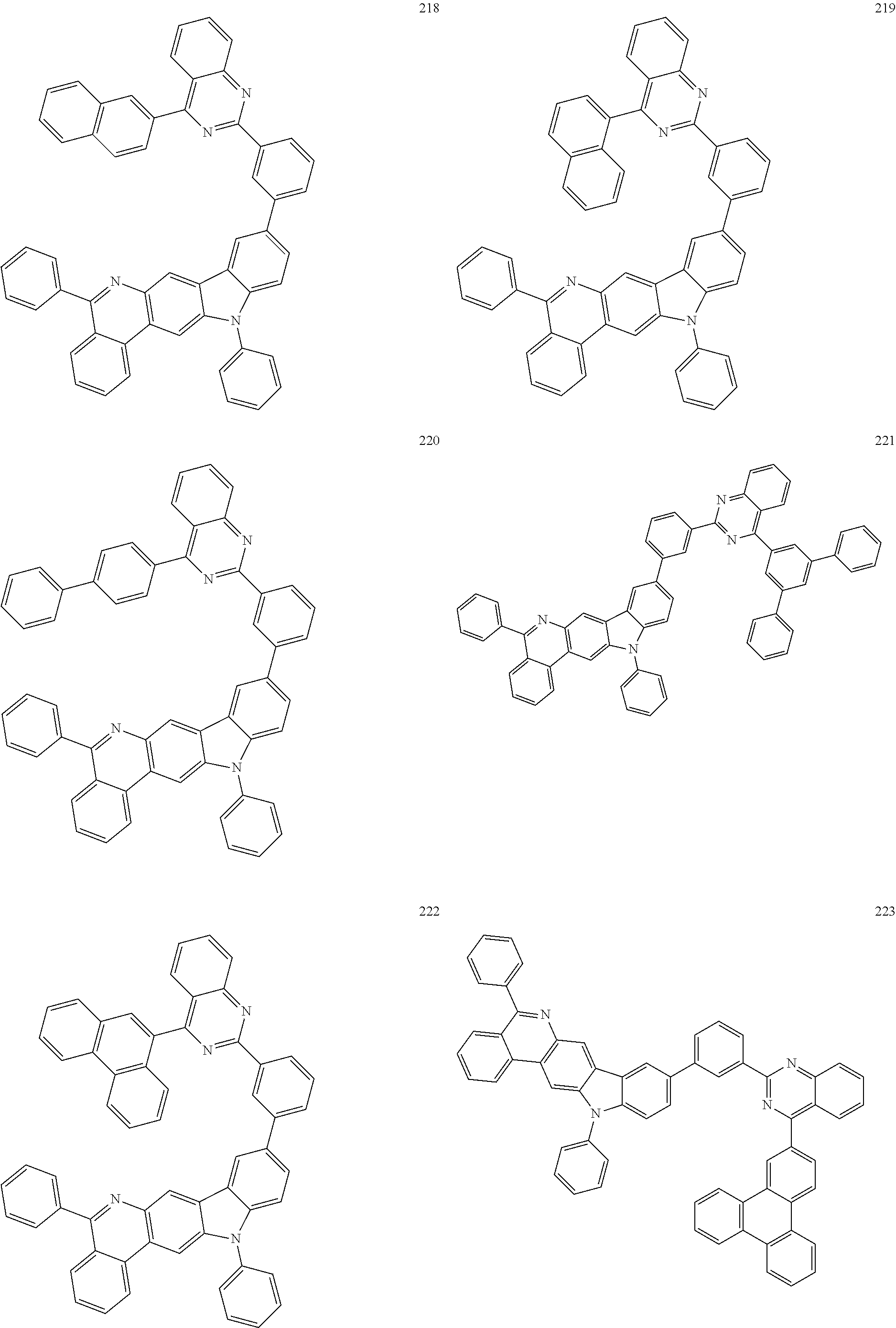

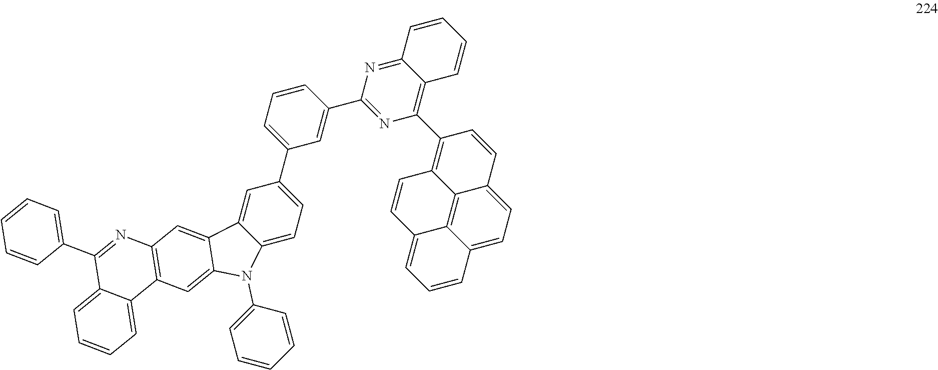 [SMILES: c1ccc(-c2nc3cc4c5cc(-c6cccc(-c7nc(-c8ccc9ccc%10cccc%11ccc8c9c%10%11)c8ccccc8n7)c6)ccc5n(-c5ccccc5)c4cc3c3ccccc23)cc1]

5. A charge transport material comprising the organic compounds of claim 1.

6. An organic light emitting diode comprising the charge transport material of claim 5.

7. The organic light emitting diode of claim 6, wherein the organic light emitting diode comprises an anode; a cathode; and one or more layered organic thin film layers interposed between the anode and the cathode,
and wherein at least one layer of the organic thin film layers comprises the charge transport material.

8. The organic light emitting diode of claim 7, wherein the charge transport material is an electron transport material or a hole transport material.

9. The organic light emitting diode of claim 7, wherein the organic thin film layer comprises at least one selected from the group consisting of a hole injecting layer, a hole transport layer, an emitting layer, an electron transport layer, and an electron injecting layer.

10. A display comprising the organic light emitting diode of claim 6.
